(19) United States
(12) Patent Application Publication
(10) Pub. No.: US 2009/0099231 A1
(43) Pub. Date: Apr. 16, 2009

(54) 3-Sulfonylamino-Pyrrolidine-2-One Derivatives as Factor Xa Inhibitors

(76) Inventors: John David Harling, Hertfordshire (GB); Nigel Stephen Watson, Hertfordshire (GB); Robert John Young, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/911,162

(22) PCT Filed: Apr. 7, 2006
(86) PCT No.: PCT/EP06/03774

§ 371 Date: Oct. 10, 2007

(30) Foreign Application Priority Data
Apr. 11, 2005 (GB) 0507287.1
Jul. 14, 2005 (GB) 0514491.0

(51) Int. Cl. A61K 31/4709; C07D 215/38; A61P 9/00

(52) U.S. Cl. 514/314; 546/171

(57) ABSTRACT

The present invention provides pyrrolidinone derivatives as Factor Xa inhibitors and pharmaceutical compositions comprising the same. The invention also relates to processes for the preparation of such compounds, and the use of such compounds in medicine, particularly in the amelioration of a clinical condition for which a Factor Xa inhibitor is indicated.

### FIELD OF THE INVENTION

The present invention relates to a novel class of chemical compounds, to processes for their preparation, to pharmaceutical compositions containing them and to their use in medicine, particularly use in the amelioration of a clinical condition for which a Factor Xa inhibitor is indicated.

### BACKGROUND OF THE INVENTION

Factor Xa is a member of the trypsin-like serine protease class of enzymes. It is a key enzyme in the coagulation cascade. A one-to-one binding of Factors Xa and Va with calcium ions and phospholipid converts prothrombin into thrombin. Thrombin plays a central role in the mechanism of blood coagulation by converting the soluble plasma protein, fibrinogen, into insoluble fibrin. The insoluble fibrin matrix is required for the stabilisation of the primary hemostatic plug. Many significant disease states are related to abnormal hemostasis. With respect to the coronary arterial vasculature, abnormal thrombus formation due to the rupture of an established atherosclerotic plaque is the major cause of acute myocardial infarction and unstable angina. Both treatment of an occlusive coronary thrombus by thrombolytic therapy and percutaneous transluminal coronary angioplasty (PTCA) are often accompanied by an acute thrombotic reclosure of the affected vessel which requires immediate resolution. With respect to the venous vasculature, a high percentage of patients undergoing major surgery in the lower extremities or the abdominal area suffer from thrombus formation in the venous vasculature which can result in reduced blood flow to the affected extremity and a pre-disposition to pulmonary embolism. Disseminated intravascular coagulopathy commonly occurs within both vascular systems during septic shock, certain viral infections and cancer and is characterised by the rapid consumption of coagulation factors and systemic coagulation which results in the formation of life-threatening thrombi occurring throughout the vasculature leading to widespread organ failure. Beyond its direct role in the formation of fibrin rich blood clots, thrombin has been reported to have profound bioregulatory effects on a number of cellular components within the vasculature and blood, (Shuman, M. A., Ann. NY Acad. Sci., 405: 349 (1986)).

A Factor Xa inhibitor may be useful in the treatment of acute vascular diseases such as acute coronary syndromes (for example primary and secondary prevention of myocardial infarction and unstable angina and treatment of prothrombotic sequalae associated with myocardial infarction or heart failure), thromboembolism including venous thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with a trial fibrillation, e.g. stroke. Factor Xa inhibitors may also be useful in preventing thrombosis and complications in patients genetically predisposed to arterial thrombosis or venous thrombosis and patients that have a disease-associated predisposition to thrombosis (e.g. type 2 diabetics). Thrombin has been reported to contribute to lung fibroblast proliferation, thus, Factor Xa inhibitors could be useful for the treatment of some pulmonary fibrotic diseases. Factor Xa inhibitors could also be useful in the treatment of tumour metastasis, by suppressing coagulation and thus preventing fibrin deposition and its concommittant facilitation of metastasis. A Factor Xa inhibitor may also have utility as an anti-inflammatory agent through its inhibition of FXa mediated activation of protease-activated receptors (PARs 1 and 2). A Factor Xa inhibitor may also have utility as an anti-atherosclerotic agent through the suppression of platelet-activation. Thrombin can induce neurite retraction and thus Factor Xa inhibitors may have potential in neurogenerative diseases such as Parkinson's and Alzheimer's disease. Factor Xa inhibitors may also have utility as anticoagulant agents in connection with the preparation, storage, fractionation or use of whole blood. They have also been reported for use in conjunction with thrombolytic agents, thus permitting the use of a lower dose of thrombolytic agent.

### DESCRIPTION OF THE INVENTION

The present invention provides at least one chemical entity chosen from compounds of formula (I):

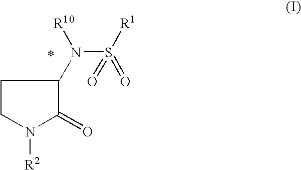

wherein:
R¹ represents a group selected from:

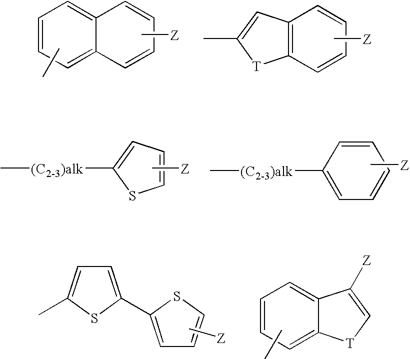

each ring of which optionally contains a further heteroatom N,
Z represents an optional substituent halogen, —C₁₋₃alkyl or —NR^{a}R^{b},
alk represents alkylene or alkenylene,
T represents S, O or NH;
R^{a} and R^{b} independently represent hydrogen or —C₁₋₃alkyl;
R² represents a group selected from:

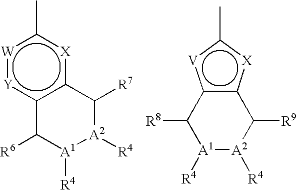

W, X and Y independently represent CH, C—R⁵ or N;
R⁵ represents halogen or C₁₋₃alkyl;
V represents NR³, S or O;
R³ represents hydrogen or C₁₋₃alkyl;
one of A¹ and A² represents N and the other represents CH;
Each R⁴, R⁶, R⁷, R⁸, R⁹ independently represents hydrogen or C₁₋₃alkyl;
R¹⁰ represents hydrogen, —C₁₋₆alkyl, —C₁₋₃alkylCONR^{a}R^{b}, —C₁₋₃alkylCO₂C₁₋₄alkyl, —CO₂C₁₋₄alkyl or —C₁₋₃alkylCO₂H;
and pharmaceutically acceptable derivative(s) thereof.

Further aspects of the invention are:
A pharmaceutical composition comprising a compound of the invention together with a pharmaceutical carrier and/or excipient.
A compound of the invention for use in therapy.
Use of a compound of the invention for the manufacture of a medicament for the treatment of a patient suffering from a condition susceptible to amelioration by a Factor Xa inhibitor.
A method of treating a patient suffering from a condition susceptible to amelioration by a Factor Xa inhibitor comprising administering a therapeutically effective amount of a compound of the invention.

In one aspect, the present invention provides at least one chemical entity chosen from compounds of formula (I):

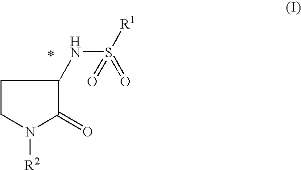

wherein:
R¹ represents a group selected from:

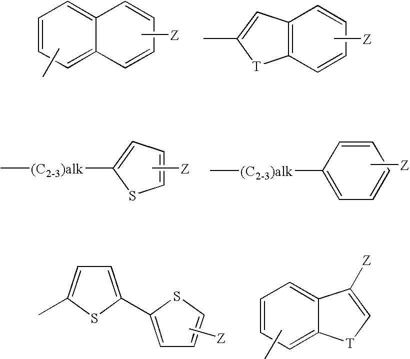

each ring of which optionally contains a further heteroatom N,
Z represents an optional substituent halogen, —C₁₋₃alkyl or —NR^{a}R^{b},
alk represents alkylene or alkenylene,
T represents S, O or NH;
R^{a} and R^{b} independently represent hydrogen or —C₁₋₃alkyl;
R² represents a group selected from:

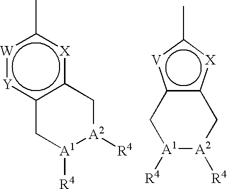

W, X and Y independently represent CH, C—R⁵ or N;
R⁵ represents halogen or C₁₋₃alkyl;
V represents NR³, S or O;
R³ represents hydrogen or C₁₋₃alkyl;
one of A¹ and A² represents N and the other represents CH;
R⁴ represents hydrogen, or when the corresponding A¹ or A² represents N then R⁴ represents hydrogen or C₁₋₃alkyl;
and pharmaceutically acceptable derivative(s) thereof.

In one aspect of the invention, R¹ represents a group selected from:

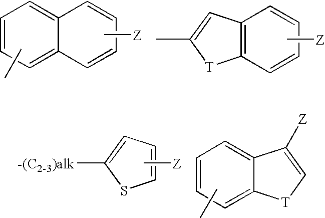

In another aspect, R¹ represents a group selected from:

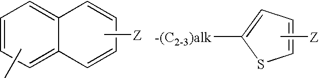

each ring of which optionally contains a further heteroatom N,
Z represents an optional substituent halogen,
alk represents alkylene or alkenylene.

In another aspect of the invention, R¹ represents a group selected from:

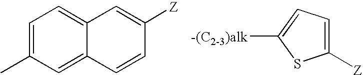

Z represents an optional substituent halogen.

In another aspect, R¹ represents a group selected from:

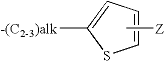

Z represents an optional substituent halogen,
alk represents alkylene or alkenylene.

In another aspect, R¹ represents a group selected from:

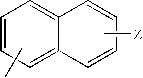

Z represents an optional substituent halogen.

In one aspect of the invention, Z represents an optional substituent halogen. In another aspect, Z represents an optional substituent Cl. In another aspect, Z represents Cl.

In one aspect of the invention, T represents S or NH. In another aspect of the invention, T represents NH.

In one aspect of the invention, R² represents a group selected from:

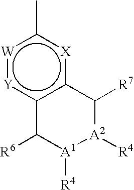

In another aspect of the invention, R² represents a group:

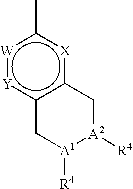

In another aspect, R² represents a group:

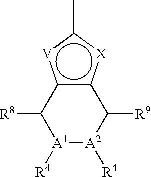

In another aspect, R² represents a group:

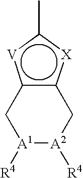

In one aspect of the invention W, X and Y independently represent CH or C-halogen or N. In another aspect of the invention W, X and Y independently represent CH or C—R⁵. In another aspect of the invention W, X and Y independently represent CH or C-halogen. In another aspect, W, X and Y independently represent CH, CF or N. In another aspect, W, X and Y independently represent CH or CF. In another aspect of the invention, at least one of W, X and Y represents CH. In another aspect, at least two of W, X and Y represent CH.

In one aspect of the invention V represents S and X represents N.

In one aspect of the invention, R⁵ represents halogen or methyl. In another aspect of the invention, R⁵ represents halogen. In another aspect of the invention, R⁵ represents F, Cl or methyl. In another aspect of the invention, R⁵ represents F.

In one aspect of the invention, A¹ represents N and A² represents CH;

In one aspect of the invention, R⁴ represents hydrogen, isopropyl or methyl. In another aspect of the invention, R⁴ represents hydrogen, or when the corresponding A¹ or A² represents N then R⁴ represents hydrogen or methyl. In another aspect of the invention, A¹(R⁴) represents —N(H)— and A²(R⁴) represents —CH(CH₃)—.

In one aspect of the invention, R⁶ represents hydrogen or methyl. In another aspect of the invention, R⁶ represents hydrogen.

In one aspect of the invention, R⁷ represents hydrogen or methyl. In another aspect of the invention, R⁷ represents hydrogen.

In one aspect of the invention, R⁸ represents hydrogen or methyl. In another aspect of the invention, R⁸ represents hydrogen.

In one aspect of the invention, R⁹ represents hydrogen or methyl. In another aspect of the invention, R⁹ represents hydrogen.

In one aspect of the invention, R¹⁰ represents hydrogen or —C₁₋₆alkyl. In another aspect of the invention, R¹⁰ represents hydrogen or methyl. In another aspect of the invention, R¹⁰ represents hydrogen.

It is to be understood that the present invention covers all combinations of the various aspects of the invention described herein above.

As used herein, the term “alkyl” means both straight and branched chain saturated hydrocarbon groups. Examples of alkyl groups include methyl (—CH₃), ethyl (—C₂H₅), propyl (—C₃H₇) and iso-propyl (—CH(CH₃)₂).

As used herein, the term “alkylene” means both straight and branched chain saturated hydrocarbon linker groups. Examples of alkylene groups include methylene (—CH₂—), ethylene (—CH₂CH₂—) and propylene (—CH₂CH₂CH₂—).

As used herein, the term “alkenylene” means both straight and branched chain unsaturated hydrocarbon linker groups, wherein the unsaturation is present only as double bonds. Examples of alkenylene groups includes ethenylene (—CH═CH—) and propenylene (—CH₂—CH═CH—).

As used herein, the term “halogen” means an atom selected from fluorine (fluoro), chlorine (chloro), bromine (bromo) and iodine (iodo).

As used herein, the term “pharmaceutically acceptable” means a compound which is suitable for pharmaceutical use.

As used herein, the term “pharmaceutically acceptable derivative”, means any pharmaceutically acceptable salt, solvate, or prodrug e.g. carbamate, or salt or solvate of such a prodrug, of a compound of formula (I), which upon administration to the recipient is capable of providing (directly or indirectly) a compound of formula (I), or an active metabolite or residue thereof. Exemplary pharmaceutically acceptable derivatives are salts, solvates and carbamates. More exemplary pharmaceutically acceptable derivatives are salts and solvates.

Suitable salts according to the invention may include those formed with both organic and inorganic acids. The term “pharmaceutically acceptable salts” includes pharmaceutically acceptable acid addition salts. These pharmaceutically acceptable salts may be prepared in situ during the final isolation and purification of the compound, or by separately reacting the purified compound in its free base form with a suitable acid. Pharmaceutically acceptable acid addition salts include those formed from mineral acids such as: hydrochloric, hydrobromic, sulphuric, phosphoric, acid; and organic acids such as: citric, tartaric, lactic, pyruvic, acetic, trifluoroacetic, succinic, oxalic, formic, fumaric, maleic, oxaloacetic, methanesulphonic, ethanesulphonic, p-toluenesulphonic, benzenesulphonic and isethionic acids. Exemplary pharmaceutically acceptable salts include those formed from hydrochloric, trifluoroacetic and formic acids. Thus, in one aspect of the invention pharmaceutically acceptable salts are formic acid salts. In another aspect of the invention pharmaceutically acceptable salts are hydrochloric acid salts.

Those skilled in the art of organic chemistry will appreciate that many organic compounds can form complexes with solvents in which they are reacted or from which they are precipitated or crystallized. These complexes are known as “solvates”. Solvates may involve non-aqueous solvents such as ethanol, isopropanol, dimethylsulfoxide (DMSO), acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Solvates of the compound of formula (I) are within the scope of the invention.

Salts and solvates of compounds of formula (I) which are suitable for use in medicine may be those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of formula (I) and their pharmaceutically acceptable salts and solvates.

The compounds of formula (I) contain a chiral (asymmetric) centre. The individual stereoisomers (enantiomers) and mixtures of these are within the scope of the present invention. Thus, the stereochemistry may be (S) or (R) at the 3-position on the 2-oxopyrrolidine ring (as indicated by the symbol *). In one aspect of the invention, the stereochemistry is (S) at the 3-position on the 2-oxopyrrolidine ring. It will understood by those skilled in the art that individual stereoisomers may be separated by standard techniques used in the art, e.g. chiral HPLC.

It will also be appreciated that compounds of the invention which exist as polymorphs, enantiomers and mixtures thereof are all contemplated to be within the scope of the present invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein an amine group is bonded to any group that, when administered to a patient, cleaves to form the amine group.

As used herein, the term “compounds of the invention” means both the compounds according to formula I and the pharmaceutically acceptable derivatives thereof. The terms “a compound of the invention” and “chemical entity” also appear herein and refer to both a compound according to formula I and its pharmaceutically acceptable derivatives.

In one aspect, chemical entities useful in the present invention may be at least one chemical entity selected from the list:
6-Chloro-N-[(3S)-2-oxo-1-(1,2,3,4-tetrahydro-6-isoquinolinyl)-3-pyrrolidinyl]-2-naphthalenesulfonamide;
(E)-2-(5-Chloro-2-thienyl)-N-[(3S)-2-oxo-1-(1,2,3,4-tetrahydro-6-isoquinolinyl)-3-pyrrolidinyl]ethenesulfonamide;
6-Chloro-N-[(3S)-2-oxo-1-(1,2,3,4-tetrahydro-6-isoquinolinyl)-3-pyrrolidinyl]-1-benzothiophene-2-sulfonamide;
3-Chloro-N-[(3S)-2-oxo-1-(1,2,3,4-tetrahydro-6-isoquinolinyl)-3-pyrrolidinyl]-1H-indole-6-sulfonamide;
N-[(3S)-2-Oxo-1-(1,2,3,4-tetrahydro-6-isoquinolinyl)-3-pyrrolidinyl]-1H-indole-6-sulfonamide;
6-Chloro-N-[(3S)-2-oxo-1-(1,2,3,4-tetrahydro-6-isoquinolinyl)-3-pyrrolidinyl]-1H-indole-2-sulfonamide;
(E)-2-(5-Chloro-2-thienyl)-N-[(3S)-1-(2-methyl-1,2,3,4-tetrahydro-6-isoquinolinyl)-2-oxo-3-pyrrolidinyl]ethenesulfonamide;
6-Chloro-N-[(3S)-1-(2-methyl-1,2,3,4-tetrahydro-6-isoquinolinyl)-2-oxo-3-pyrrolidinyl]-1-benzothiophene-2-sulfonamide;
5-Chloro-N-[(3S)-2-oxo-1-(1,2,3,4-tetrahydro-6-isoquinolinyl)-3-pyrrolidinyl]-1-benzothiophene-2-sulfonamide;
5′-Chloro-N-[(3S)-2-oxo-1-(1,2,3,4-tetrahydro-6-isoquinolinyl)-3-pyrrolidinyl]-2,2′-bithiophene-5-sulfonamide;
6-Chloro-N-[(3S)-2-oxo-1-(1,2,3,4-tetrahydro-7-isoquinolinyl)-3-pyrrolidinyl]-2-naphthalenesulfonamide;
2-(5-Chloro-2-thienyl)-N-[(3S)-2-oxo-1-(1,2,3,4-tetrahydro-7-isoquinolinyl)-3-pyrrolidinyl]ethanesulfonamide;
(E)-2-(5-Chloro-2-thienyl)-N-[(3S)-2-oxo-1-(1,2,3,4-tetrahydro-7-isoquinolinyl)-3-pyrrolidinyl]ethenesulfonamide;
3-Chloro-N-[(3S)-2-oxo-1-(1,2,3,4-tetrahydro-7-isoquinolinyl)-3-pyrrolidinyl]-1H-indole-6-sulfonamide;
N-[(3S)-2-Oxo-1-(1,2,3,4-tetrahydro-7-isoquinolinyl)-3-pyrrolidinyl]-1H-indole-6-sulfonamide;
6-Chloro-N-[(3R)-2-oxo-1-(1,2,3,4-tetrahydro-6-isoquinolinyl)-3-pyrrolidinyl]-2-naphthalenesulfonamide;
6-Chloro-N-[(3S)-1-(5-fluoro-1,2,3,4-tetrahydro-6-isoquinolinyl)-2-oxo-3-pyrrolidinyl]-2-naphthalenesulfonamide;
(E)-2-(5-Chloro-2-thienyl)-N-[(3S)-1-(5-fluoro-1,2,3,4-tetrahydro-6-isoquinolinyl)-2-oxo-3-pyrrolidinyl]ethenesulfonamide;
3-Chloro-N-[(3S)-1-(5-fluoro-1,2,3,4-tetrahydro-6-isoquinolinyl)-2-oxo-3-pyrrolidinyl]-1H-indole-6-sulfonamide;
6-Chloro-N-[1-(7-fluoro-1,2,3,4-tetrahydro-6-isoquinolinyl)-2-oxo-3-pyrrolidinyl]-2-naphthalenesulfonamide;
6-Chloro-N-[(3S)-1-(7-fluoro-1,2,3,4-tetrahydro-6-isoquinolinyl)-2-oxo-3-pyrrolidinyl]-1-benzothiophene-2-sulfonamide;
(E)-2-(5-Chloro-2-thienyl)-N-[(3S)-1-(7-fluoro-1,2,3,4-tetrahydro-6-isoquinolinyl)-2-oxo-3-pyrrolidinyl]ethenesulfonamide;
3-Chloro-N-[(3S)-1-(7-fluoro-1,2,3,4-tetrahydro-6-isoquinolinyl)-2-oxo-3-pyrrolidinyl]-1H-indole-6-sulfonamide;
6-Chloro-N-[(3S)-1-(7-chloro-1,2,3,4-tetrahydro-6-isoquinolinyl)-2-oxo-3-pyrrolidinyl]-2-naphthalenesulfonamide;
(E)-N-[(3S)-1-(7-Chloro-1,2,3,4-tetrahydro-6-isoquinolinyl)-2-oxo-3-pyrrolidinyl]-2-(5-chloro-2-thienyl)ethenesulfonamide;
3-Chloro-N-[(3S)-1-(7-chloro-1,2,3,4-tetrahydro-6-isoquinolinyl)-2-oxo-3-pyrrolidinyl]-1H-indole-6-sulfonamide;
6-Chloro-N-[(3S)-1-(7-methyl-1,2,3,4-tetrahydro-6-isoquinolinyl)-2-oxo-3-pyrrolidinyl]-2-naphthalenesulfonamide;
(E)-2-(5-Chloro-2-thienyl)-N-[(3S)-1-(7-methyl-1,2,3,4-tetrahydro-6-isoquinolinyl)-2-oxo-3-pyrrolidinyl]ethenesulfonamide;
3-Chloro-N-[(3S)-1-(7-methyl-1,2,3,4-tetrahydro-6-isoquinolinyl)-2-oxo-3-pyrrolidinyl]-1H-indole-6-sulfonamide;
6-Chloro-N-[(3S)-1-(1-methyl-1,2,3,4-tetrahydro-6-isoquinolinyl)-2-oxo-3-pyrrolidinyl]-2-naphthalenesulfonamide;
(E)-2-(5-Chloro-2-thienyl)-N-[(3S)-1-(1-methyl-1,2,3,4-tetrahydro-6-isoquinolinyl)-2-oxo-3-pyrrolidinyl]ethenesulfonamide;
(E)-2-(5-Chloro-2-thienyl)-N-[(3S)-1-(3-methyl-1,2,3,4-tetrahydro-6-isoquinolinyl)-2-oxo-3-pyrrolidinyl]ethenesulfonamide;
(E)-2-(5-Chloro-2-thienyl)-N-methyl-N-[(3S)-2-oxo-1-(1,2,3,4-tetrahydro-6-isoquinolinyl)-3-pyrrolidinyl]ethenesulfonamide;
(E)-2-(5-Chloro-2-thienyl)-N-[(3S)-2-oxo-1-(4,5,6,7-tetrahydro[1,3]thiazolo[5,4-c]pyridin-2-yl)-3-pyrrolidinyl]ethenesulfonamide;
5-Chloro-N-[(3S)-2-oxo-1-(1,2,3,4-tetrahydro-6-isoquinolinyl)-3-pyrrolidinyl]-1H-indole-2-sulfonamide;
(E)-2-(4-Chlorophenyl)-N-[(3S)-1-(2-methyl-1,2,3,4-tetrahydro-6-isoquinolinyl)-2-oxo-3-pyrrolidinyl]ethenesulfonamide;
6-Chloro-N-[(3S)-1-(3-methyl-1,2,3,4-tetrahydro-6-isoquinolinyl)-2-oxo-3-pyrrolidinyl]-2-naphthalenesulfonamide;
   and pharmaceutically acceptable derivatives thereof.

Compounds of the invention may show advantageous properties, they may be more efficacious, may show greater selectivity, may have fewer side effects, may have a longer duration of action, may be more bioavailable by the preferred route, or may have other more desirable properties than similar known compounds.

The compounds of formula (I) are Factor Xa inhibitors and as such are useful in the treatment of clinical conditions susceptible to amelioration by administration of a Factor Xa inhibitor. Such conditions may include acute vascular diseases such as acute coronary syndromes (for example primary and secondary prevention of myocardial infarction and unstable angina and treatment of prothrombotic sequalae associated with myocardial infarction or heart failure), thromboembolism including venous thromboembolism, a cute vessel closure associated with thrombolytic therapy and percutaneous transluminal coronary angioplasty (PTCA), transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke; in preventing thrombosis and complications in patients genetically predisposed to arterial thrombosis or venous thrombosis and patients that have a disease-associated predisposition to thrombosis (e.g. type 2 diabetics); the treatment of pulmonary fibrosis; the treatment of tumour metastasis; inflammation; atherosclerosis; neurogenerative disease such as Parkinson's and Alzheimer's diseases; Kasabach Merritt Syndrome; Haemolytic uremic syndrome; endothelial dysfunction; as anti-coagulants for extracorporeal blood in for example, dialysis, blood filtration, bypass, and blood product storage; and in the coating of invasive devices such as prostheses, artificial valves and catheters in reducing the risk of thrombus formation.

Accordingly, one aspect of the present invention provides a compound of formula (I) and/or a pharmaceutically acceptable derivative thereof for use in medical therapy, for example, for use in the amelioration of a clinical condition in a mammal, including a human, for which a Factor Xa inhibitor is indicated.

In another aspect, the invention provides a method for the treatment and/or prophylaxis of a condition susceptible to amelioration by a Factor Xa inhibitor in a mammal, including a human, which method comprises administering to the subject an effective amount of a compound of formula (I) or a pharmaceutically acceptable derivative thereof.

In another aspect, the present invention provides the use of a compound of formula (I) and/or a pharmaceutically acceptable derivative thereof, for the manufacture of a medicament for the treatment and/or prophylaxis of a condition susceptible to amelioration by a Factor Xa inhibitor.

In one aspect of the invention, the condition susceptible to amelioration by a Factor Xa inhibitor is selected from treatment of acute vascular diseases such as acute coronary syndromes (for example primary and secondary prevention of myocardial infarction and unstable angina and treatment of prothrombotic sequalae associated with myocardial infarction or heart failure), thromboembolism including venous thromboembolism, acute vessel closure associated with thrombolytic therapy and percutaneous transluminal coronary angioplasty, transient ischemic attacks, pulmonary embolism, deep vein thrombosis, peripheral arterial occlusion, prevention of vessel luminal narrowing (restenosis), and the prevention of thromboembolic events associated with atrial fibrillation, e.g. stroke.

In another aspect, the condition susceptible to a melioration by a Factor X a inhibitor is selected from acute coronary syndromes (for example primary and secondary prevention of myocardial infarction and unstable angina and treatment of prothrombotic sequalae associated with myocardial infarction or heart failure), pulmonary embolism, deep vein thrombosis and the prevention of thromboembolic events associated with atrial fibrillation, e.g. stroke.

It will be appreciated that reference to treatment includes acute treatment or prophylaxis as well as the alleviation of established symptoms.

Within the context of the present invention, the terms describing the indications used herein are classified in the The Merck Manual of Diagnosis and Therapy, 17^{th} Edition and/or the International Classification of Diseases, 10th Edition (ICD-10). The various subtypes of the disorders mentioned herein are contemplated as part of the present invention.

While it is possible that, for use in therapy, a compound of the present invention may be administered as the raw chemical, the active ingredient may also be presented as a pharmaceutical formulation.

In a further aspect, the invention provides a pharmaceutical composition comprising at least one compound of formula (I) and/or a pharmaceutically acceptable derivative thereof in association with at least one pharmaceutically acceptable carrier and/or excipient. The carrier and/or excipient must be “acceptable” in the sense of being compatible with the other ingredients of the formulation and not deleterious to the recipient thereof.

In another aspect, the invention provides a pharmaceutical composition comprising, as active ingredient, at least one compound of formula (I) and/or a pharmaceutically acceptable derivative thereof in association with a pharmaceutically acceptable carrier and/or excipient for use in therapy, and for example in the treatment of human or animal subjects suffering from a condition susceptible to amelioration by a Factor Xa inhibitor.

There is further provided by the present invention a process of preparing a pharmaceutical composition, which process comprises mixing at least one compound of formula (I) and/or a pharmaceutically acceptable derivative thereof, together with at least one pharmaceutically acceptable carrier and/or excipient.

The compounds for use according to the present invention may be formulated for oral, buccal, parenteral, topical, rectal or transdermal administration or in a form suitable for administration by inhalation or insufflation (either through the mouth or the nose).

For oral administration, the pharmaceutical compositions may take the form of, for example, tablets or capsules prepared by conventional means with pharmaceutically acceptable excipients such as binding agents (e.g. pregelatinised maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycollate); or wetting agents (e.g. sodium lauryl sulfate). The tablets may be coated by methods well known in the art. Liquid preparations for oral administration may take the form of, for example, solutions, syrups or suspensions or they may be presented as a dry product for constitution with water or other suitable vehicles before use. Such liquid preparations may be prepared by conventional means with pharmaceutically acceptable additives such as suspending agents (e.g. sorbitol syrup, cellulose derivatives or hydrogenated edible fats); emulsifying a gents (e.g. lecithin or acacia); non-aqueous vehicles (e.g. almond oil, oily esters, ethyl alcohol or fractionated vegetable oils); and preservatives (e.g. methyl or propyl-p-hydroxybenzoates or sorbic acid). The preparations may also contain buffer salts, flavouring, colouring and sweetening agents as appropriate.

Preparations for oral administration may be suitably formulated to give controlled/extended release of the active compound.

For buccal administration the compositions may take the form of tablets or lozenges formulated in a conventional manner.

The compounds according to the present invention may be formulated for parenteral administration by injection, e.g. by bolus injection or continuous infusion. Formulations for injection may be presented in unit dosage form, e.g. in ampoules or in multi-dose containers, with an added preservative. The compositions may take such forms as suspensions, solutions or emulsions in oily or aqueous vehicles, and may contain formulatory agents such as suspending, stabilising and/or dispersing agents. Alternatively, the active ingredient may be in powder form for constitution with a suitable vehicle, e.g. sterile pyrogen-free water, before use.

The compounds according to the present invention may be formulated for topical administration by insufflation and inhalation. Examples of types of preparation for topical administration include sprays and aerosols for use in an inhaler or insufflator.

Powders for external application may be formed with the aid of any suitable powder base, for example, lactose, talc or starch. Spray compositions may be formulated as aqueous solutions or suspensions or as aerosols delivered from pressurised packs, such as metered dose inhalers, with the use of a suitable propellant.

The compounds according to the present invention may also be formulated in rectal compositions such as suppositories or retention enemas, e.g. containing conventional suppository bases such as cocoa butter or other glycerides.

In addition to the formulations described previously, the compounds may also be formulated as a depot preparation. Such long acting formulations may be administered by implantation (for example subcutaneously, transcutaneously or intramuscularly) or by intramuscular injection. Thus, for example, the compounds according to the present invention may be formulated with suitable polymeric or hydrophobic materials (for example as an emulsion in an acceptable oil) or ion exchange resins or as sparingly soluble derivatives, for example, as a sparingly soluble salt.

A proposed dose of the compounds according to the present invention for administration to a human (of approximately 70 kg body weight) is 0.1 mg to 1 g, such as 1 mg to 500 mg of the active ingredient per unit dose, expressed as the weight of free base. The unit dose may be administered, for example, 1 to 4 times per day. It will be appreciated that it may be necessary to make routine variations to the dosage depending on the age and weight of the patient as well as the severity of the condition to be treated. The dosage may also depend on the route of administration. The precise dose and route of administration will ultimately be at the discretion of the attendant physician or veterinarian.

The compounds of formula (I) may also be used in combination with other therapeutic agents. The invention thus provides, in a further aspect, a combination comprising at least one compound of formula (I) and/or a pharmaceutically acceptable derivative thereof together with one or more further therapeutic agent(s).

When at least one chemical entity chosen from compounds of formula (I) and/or a pharmaceutically acceptable derivative thereof is used in combination with a second therapeutic agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may be used in combination with other antithrombotic drugs (such as thrombin inhibitors, thromboxane receptor antagonists, prostacyclin mimetics, phosphodiesterase inhibitors, fibrinogen antagonists, thrombolytic drugs such as tissue plasminogen activator and streptokinase, non-steroidal anti-inflammatory drugs such as aspirin, and the like), anti-hypertensive agents (such as angiotensin-converting enzyme inhibitors, angiotensin-II receptor antagonists, ACE/NEP inhibitors, β-blockers, calcium channel blockers, PDE inhibitors, aldosterone blockers), anti-atherosclerotic/dyslipidaemic agents (such as HMG-CoA reductase inhibitors) and anti-arrhythmic agents.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention. The individual components of such combinations may be administered either sequentially or simultaneously in separate or combined pharmaceutical formulations by any convenient route.

When administration is sequential, either the Factor Xa inhibitor or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

The chemical entities chosen from compounds of formula (I) and/or pharmaceutically acceptable derivatives thereof may be prepared by the processes described hereinafter, said processes constituting a further aspect of the invention. In the following description, the groups are as defined above for compounds of formula (I) unless otherwise stated.

According to a further aspect of the present invention, there is provided a process (A) for preparing compounds of formula (I) which comprises reacting compounds of formula (II) or an acid addition salt thereof with compounds of formula (III) where V is a suitable leaving group, such as a halide, e.g. chloride. When the free base of a compound of formula (II) is used, the reaction is conveniently carried out in the presence of a base, e.g. pyridine, and in a suitable solvent, e.g. acetonitrile (MeCN), suitably at 0° C. to room temperature. In compounds of formula (II), P¹ represents an optional amine protecting group. Where P¹ is a protecting group, e.g. t-butyloxycarbonyl (Boc), the reaction of compounds of formula (II) and compounds of formula (III) is followed by removal of the protecting group under standard conditions. For example, where P¹ represents Boc, removal of the protecting group may be effected under acidic conditions, using a source of HCl, for example acetyl chloride in methanol (MeOH).

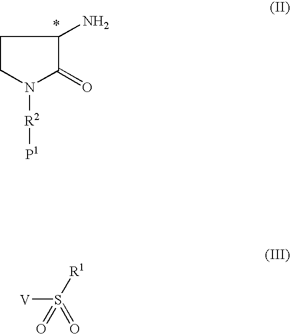

Where R¹ represents a nitrogen containing heterocycle, e.g. an indole, R¹ may be protected with a suitable amine protecting group which may be removed under standard conditions after the reaction between compounds of formula (II) with compounds of formula (III). For example, where the protecting group is tris(1-methylethyl)silyl this may be removed by acid deprotection, e.g. by treatment with acetic acid in the presence of a suitable solvent, e.g. tetrahydrofuran (THF). For example, where the protecting group is Boc, this may be removed by acid deprotection, e.g. by treatment with HCl in MeOH, or HCl in 1,4-dioxane.

Compounds of formula (III) are known compounds or may be prepared by methods known in the literature or processes known to those skilled in the art.

Compounds of formula (II) may be prepared from compounds of formula (IV) by removal of the protecting group P², under standard conditions. For example, where P² represents benzyloxycarbonyl (Cbz), removal of the protecting group may be effected by reaction with hydrogen in the presence of a metal catalyst, e.g. palladium/C or palladium hydroxide, in a suitable solvent e.g. ethanol (EtOH). For example, where P² represents Boc, removal of the protecting group may be effected under acidic conditions, using a source of HCl, for example acetyl chloride in MeOH.

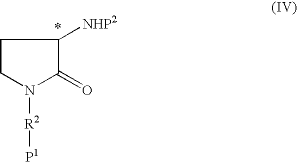

Compounds of formula (IV) may be prepared from compounds of formula (V):

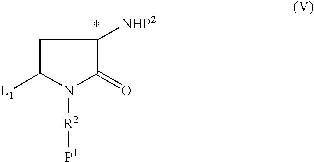

by cyclisation where L₁ represents a suitable group, e.g. hydroxyl, SMe. For example, when L₁ represents SMe by treatment with a compound capable of converting sulfur in the SMe moiety to a sulfonium salt, e.g. SMeRX, by reaction with RX (e.g. MeI), in a suitable solvent, e.g. MeCN, followed by ring closure. The ring closure may be performed with caesium carbonate (Cs₂CO₃) in a suitable solvent, e.g. MeCN, suitably at elevated temperature, such as 50-70° C. For example, where L₁ is a hydroxyl group, P¹ may be absent, A¹ or A² within R² represents N and the corresponding R⁴ represents C₁₋₃alkyl, the ring closure may be performed by treatment with a mixture of (i) aryl or alkyl phosphine, e.g. tri-n-butylphosphine, and (ii) a suitable azodicarboxylate derivative, e.g. di-tert-butyl azodicarboxylate, in a suitable solvent, e.g. THF, suitably at room temperature.

Compounds of formula (V) in which L₁ represents SMe may be prepared by reacting compounds of formula (VI) with compounds of formula (VII)

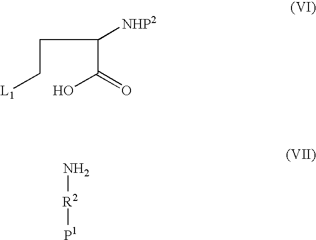

in the presence of a coupling agent, for example 2-(7-azabenzotriazole-1-yl)-1,1,3,3-tetramethyluronium hexafluorophosphate (HATU) and a base, e.g. N,N-diisopropylethylamine (DIPEA), in a suitable solvent, e.g. dichloromethane (DCM), suitably at 0° C. to room temperature.

Compounds of formula (VI) are known compounds or may be prepared by methods known in the literature or processes known to those skilled in the art.

Compounds of formula (VII) are known compounds or may be prepared by methods known in the literature or processes known to those skilled in the art.

Compounds of formula (V), where L₁ is a hydroxyl group, may be prepared by reacting compounds of formula (VIII) with compounds of formula (VII):

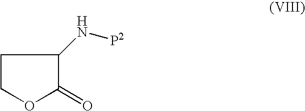

wherein P² represents a protecting group. The reaction is conveniently carried out by addition of a suitable activating agent, e.g. trimethylaluminium, to compounds of formula (VII) in a suitable solvent, e.g. DCM, under an inert atmosphere, e.g. nitrogen, suitably at room temperature followed by addition of a compound of formula (VIII) in a compatible solvent, e.g. DCM.

Compounds of formula (VIII) are known in the art or may be prepared from compounds of formula (IX) where HA is a suitable acid, e.g. hydrochloric acid, using methods well known to those skilled in the art. See, for example, “Protective groups in organic synthesis” by T. W. Greene and P. G. M. Wuts (John Wiley & sons 1991) or “Protecting Groups” by P. J. Kocienski (Georg Thieme Verlag 1994).

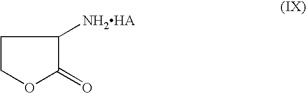

Compounds of formula (IX) are known compounds or may be prepared by methods known in the literature or processes known to those skilled in the art.

Compounds of formula (VII) having the formula (X) where B represents halogen or C₁₋₃alkyl:

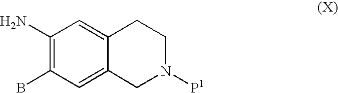

may be prepared from compounds of formula (XI):

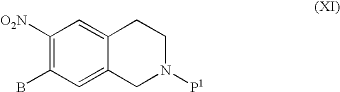

by reaction with hydrogen in the presence of a metal catalyst, for example, palladium/C, in a suitable solvent, e.g. EtOH, or tin (II) chloride dihydrate, in a suitable solvent, e.g. ethyl acetate, suitably at room temperature. In one aspect of the invention B represents halogen.

Compounds of formula (XI) may be prepared from compounds of formula (XII):

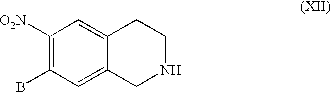

by protection with a suitable amine protecting group under standard conditions. For example, where P¹ represents Boc, by treatment with di-tert butyl carboxylate (Boc₂O) in the presence of a suitable base, e.g. triethylamine (Et₃N), and in a suitable solvent, e.g. dioxane, optionally in the presence of water.

Compounds of formula (XII) may be prepared from compounds of formula (XIII):

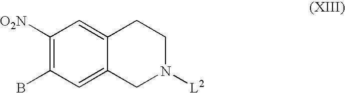

where L² represents a group, e.g. trifluoroacetyl, by removal of the group L², under standard conditions. For example, where L² represents trifluoroacetyl, removal of the protecting group may be effected under acidic conditions, for example using a source of HCl, for example, acetyl chloride in the presence of MeOH.

Compounds of formula (XIII) may be prepared from compounds of formula (XIV):

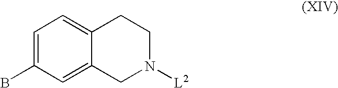

by nitration, for example using potassium nitrate, under acidic conditions e.g. H₂SO₄, suitably at 0 to 5° C. This may optionally be followed by recrystallisation to obtain a compound of formula (XIII) with a higher degree of purity.

Compounds of formula (XIV) may be prepared from compounds of formula (XV)

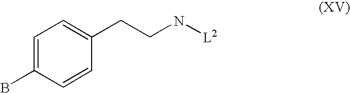

where L² represents an activating group, by ring closure. The ring closure may be performed using paraformaldehyde under acidic conditions, e.g. in an acetic acid and sulphuric acid mixture, suitably at room temperature.

Compounds of formula (XV) may be prepared from compounds of formula (XVI):

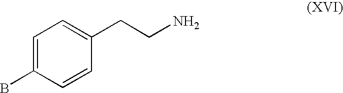

by reaction with an activating agent, e.g. trifluoroacetic anhydride, in the presence of a suitable base, e.g. triethylamine, and in a suitable solvent, e.g. DCM, suitably at −5 to 0° C.

Compounds of formula (XVI) are known compounds and may be prepared by methods known in the literature or processes known to those skilled in the art.

Compounds of formula (VII) having the formula (XVII) where B represents halogen or C₁₋₃alkyl:

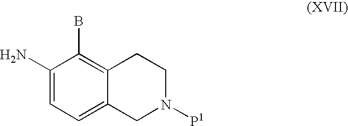

may be prepared from compounds of formula (XVIII):

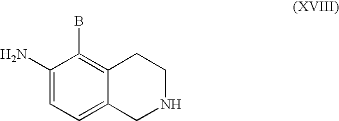

by protection with a suitable amine protecting group. For example, where P¹ represents Boc, by treatment with Boc₂O in a suitable solvent, e.g. DCM, suitably at room temperature. In one aspect of the invention, B represents halogen.

Compounds of formula (XVIII) may be prepared from compounds of formula (XIX):

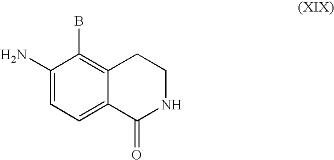

by reduction with a hydride source, e.g. borane in a suitable solvent, e.g. THF, suitably at reflux.

Compounds of formula (XIX) may be prepared from compounds of formula (XX):

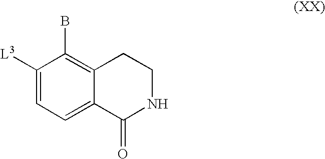

where L³ represents a suitable leaving group, such as halide, e.g. fluoro, by treatment with ammonia in a suitable solvent, e.g. methanol, suitably at elevated temperature, e.g. 100-200° C.

Compounds of formula (XX) may be prepared from compounds of formula (XXI):

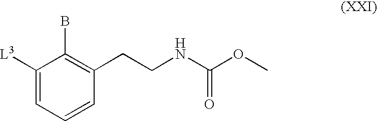

by ring closure, for example in the presence of polyphosphoric acid (PPA) and phosphorus pentoxide, suitably at 120-160° C. temperature.

Compounds of formula (XXI) may be prepared from compounds of formula (XXII):

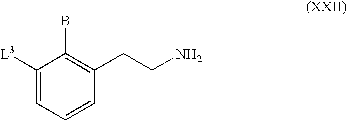

by treatment with an activating agent, for example CH₃OCOCl, in a suitable solvent such as DCM, in the presence of a base, e.g. pyridine, suitably at 0° C. to room temperature.

Compounds of formula (XXII) may be prepared from compounds of formula (XXIII):

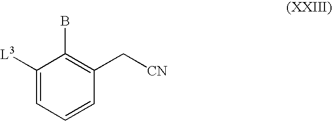

by reduction with a hydride source, e.g. borane in a suitable solvent, e.g. THF, suitably at reflux.

Compounds of formula (XXIII) are known compounds or may be prepared by methods known in the literature or processes known to those skilled in the art.

Compounds of formula (I) where R¹⁰ is a substituent other than hydrogen may be prepared by reacting a compound of formula (I) with a P¹ protecting group as appropriate where R¹⁰ is hydrogen with a compound of formula (XXIV):

R¹⁰-D (XXIV)

wherein R¹ and R¹⁰ are defined as above and D is a suitable leaving group such as a halide, e.g. iodide, followed by removal of the protecting group P¹ as appropriate. The reaction is effected in a suitable organic solvent, e.g. THF, DMF, MeCN in the presence of a base, e.g. LiHMDS (lithium hexamethyldisilylamide), potassium carbonate or sodium carbonate at a temperature range from −78° C. to +50° C., preferably −78° C. to room temperature. Furthermore, it will appreciated that the substituent R¹⁰, other than hydrogen, may be introduced at various intermediate stages by methods well known to those skilled in the art.

Compounds of formula (VII) where R²—P¹ represents:

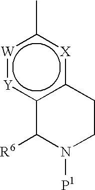

and R⁶ represents C₁₋₃alkyl, may be prepared from compounds of formula (XXV) where R⁶ represents C₁₋₃alkyl:

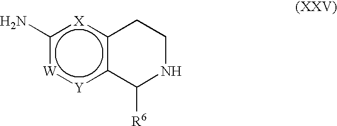

by protection with a suitable amine protecting group under standard conditions. For example, where P¹ represents Boc, by treatment with di-tert butyl dicarbonate (Boc₂O) in the presence of a suitable base, e.g. triethylamine (Et₃N), and in a suitable solvent, e.g. dioxane, optionally in the presence of water, suitably at room temperature. In one aspect of the invention, W, X and Y represent CH.

Compounds of formula (XXV) where R⁶ represents C₁₋₃alkyl, may be prepared from compounds of formula (XXVI):

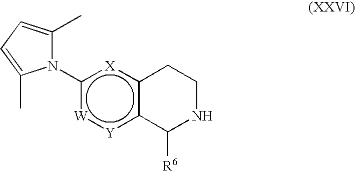

by reaction with hydroxylamine hydrochloride under standard conditions for example in the presence of an inorganic base e.g. potassium hydroxide in aqueous ethanol suitably at reflux.

Compounds of formula (XXVI) where R⁶ represents C₁₋₃alkyl, may be prepared from compounds of formula (XXVII):

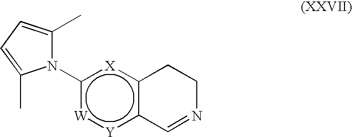

by reaction with an alkylmagnesium halide, e.g. methyl magnesium bromide, in the presence of a Lewis acid e.g. boron trifluoride etherate in a suitable solvent e.g. THF at −78° C. to room temperature. The reaction may be carried out with isolation of the Lewis acid complex (XXVIIa). Compounds of formula (XXVI) may be prepared from compounds of formula (XXVIIa) by reaction with the corresponding alkylmagnesium halide, e.g. methyl magnesium bromide, in a suitable solvent e.g. THF suitably at elevated temperature e.g. 50-70° C.

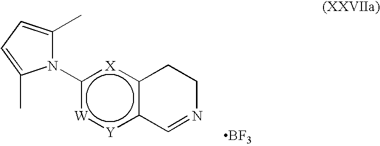

Compounds of formula (XXVII) may be prepared from compounds of formula (XXVIII):

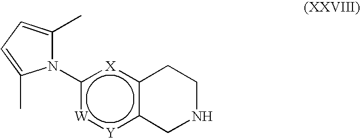

by oxidation, for example using manganese dioxide in a suitable solvent, e.g. DCM, suitably at room temperature.

Compounds of formula (XXVIII) may be prepared from compounds of formula (XXIX):

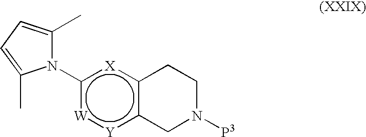

where P³ represents an amine protecting group e.g. trifluoroacetyl, by removal of the group P³ under standard conditions. For example when P³ represents trifluoroacetyl, removal of the protecting group may be effected under basic conditions, for example using potassium carbonate in aqueous methanol suitably at reflux.

Compounds of formula (XXIX) may be prepared from compounds of formula (VII) where R² represents:

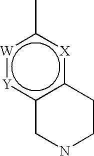

by reaction with 2,5-hexanedione in the presence of an organic acid, e.g. 4-toluenesulfonic acid hydrate, in a suitable solvent, e.g. toluene, suitably at reflux with removal of water, for example in a Dean-Stark separator. In one aspect of the invention, W, X and Y represent CH.

Compounds of formula (VII) where R² represents:

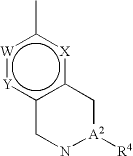

and A²(R⁴) represents —CH(C₁₋₃alkyl)- may be prepared from compounds of formula (XXX) where A²(R⁴) represents —CH(C₁₋₃alkyl)-:

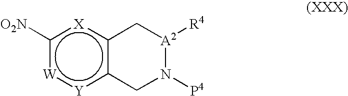

by reaction with hydrogen in the presence of a metal catalyst, for example, palladium/C in a suitable solvent, e.g. EtOH, at 40-50 psi and suitably at room temperature to 50° C. In one aspect of the invention, W, X and Y represent CH.

Compounds of formula (XXX) may be prepared from compounds of formula (XXXI):

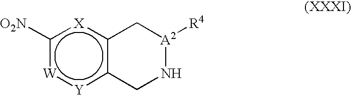

by protection with a suitable amine protecting group under standard conditions. For example where P⁴ represents Boc, by treatment with di-tert-butyl carbonate in the presence of a suitable base e.g. pyridine and in a suitable solvent e.g. DCM, suitably at room temperature.

Compounds of formula (XXXI) may be prepared from compounds of formula (XXXII):

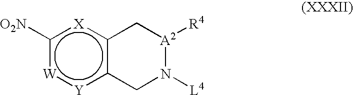

where L⁴ represents a suitable activating group, e.g. methanesulfonyl, by removal of the group L⁴ under standard conditions. For example when L⁴ represents methanesulfonyl, removal of the protecting group may be effected under acidic conditions, for example using aqueous HBr suitably at elevated temperature, e.g. 70-90° C.

Compounds of formula (XXXII) may be prepared from compounds of formula (XXXIII) where L⁴ represents a suitable activating group:

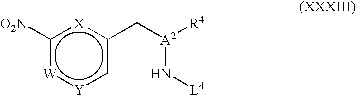

by ring closure. The ring closure may be performed using paraformaldehyde under acidic conditions e.g. in an acetic acid and sulphuric acid mixture suitably at elevated temperature e.g. 40-60° C.

Compounds of formula (XXXIII) may be prepared from compounds of formula (XXXIV):

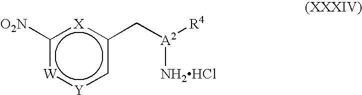

by reaction with an activating agent e.g. methanesulfonic anhydride or methanesulfonylchloride in the presence of a suitable base e.g. pyridine or triethylamine in a suitable solvent e.g. MeCN or DCM suitably at room temperature.

Compounds of formula (XXXIV) are known compounds and may be prepared by methods known in the literature or processes known to those skilled in the art.

Compounds of formula (VII) where R²—P¹ represents:

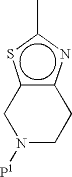

may be prepared from compounds of formula (XXXV):

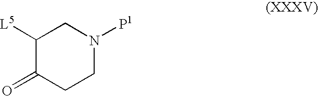

where L⁵ represents a leaving group suitably halogen, e.g. bromo, by reaction with thiourea in a suitable solvent, e.g. acetone, in the presence of a suitable base, e.g. Et₃N, suitably at room temperature.

Compounds of formula (XXXV) are known compounds and may be prepared by methods known in the literature or processes known to those skilled in the art.

It will be appreciated by those skilled in the art that compounds of formula (I) or a solvate thereof may be synthesized from appropriate intermediates via solid phase chemistry processes.

It will be appreciated by persons skilled in the art that compounds of formula (I) may be prepared by interconversion, utilising other compounds of formula (I), which are optionally protected by standard protecting groups, as precursors. For instance, compounds of formula (I) where A¹ or A² is N and the attached R⁴ is hydrogen, may be converted into compounds of formula (I) where R⁴ is C₁₋₃alkyl by N-alkylation. Also, compounds of formula (I) where V is NR³ and R³ is hydrogen, may be converted into compounds of formula (I) where V is N(R³) and R³ is C₁₋₃alkyl by N-alkylation. For example, N-alkylation may be carried out by treatment with paraformaldehyde under acidic conditions, e.g. formic acid, in a suitable solvent e.g. chloroform, suitably under reflux. Alternatively, alkylation may be carried out by treatment with tetramethylammonium triacetoxyborohydride under acidic conditions, e.g. acetic acid, in a suitable solvent e.g. acetone.

The various general methods described above may be useful for the introduction of the desired groups at any stage in the stepwise formation of the required compound, and it will be appreciated that these general methods can be combined in different ways in such multi-stage processes. The sequence of the reactions in multi-stage processes should of course be chosen so that the reaction conditions used do not affect groups in the molecule which are desired in the final product. For example, those skilled in the art will appreciate that, N-alkylation of A¹, A² or V may also be carried out on compounds of formula (IV) by removal of the protecting group and selective N-alkylation as described above, or at other convenient stages.

Those skilled in the art will appreciate that in the preparation of compounds of formula (I) and pharmaceutically acceptable derivatives thereof it may be beneficial to protect certain stages from light.

Those skilled in the art will appreciate that in the preparation of compounds of formula (I) and pharmaceutically acceptable derivatives thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl).

Various intermediate compounds used in the above-mentioned process, including but not limited to certain compounds of formulae (II), (IV) and (V) constitute a further aspect of the present invention.

The present invention will now be further illustrated by the accompanying examples which should not be construed as limiting the scope of the invention in any way.

All publications, including but not limited to patents and patent applications, cited in this specification are herein incorporated by reference as if each individual publication were specifically and individually indicated to be incorporated by reference herein as though fully set forth.

### EXAMPLES

### Abbreviations

### THF Tetrahydrofuran

### MeCN Acetonitrile

### DCM Dichloromethane

### DIPEA N,N-Diisopropylethylamine

Boc t-butyloxycarbonyl
br broad
Cbz benzyloxycarbonyl
d doublet
dd doublet of doublets
eq equivalents
EtOH ethanol
HATU 2-(7-Azabenzotriazole-1-yl)-1,1,3,3-tetramethyluronium hexafluorophosphate
m multiplet

### MeOH Methanol

q quartet
obs obscured
singlet
t triplet
min minutes
h hours

### Intermediate 1

### 1,1-Dimethylethyl 6-[(N-{[(phenylmethyl)oxy]carbonyl}-L-methionyl)amino]-3,4-dihydro-2(1H)-isoquinolinecarboxylate

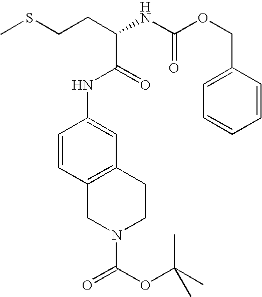

1,1-Dimethylethyl 6-amino-3,4-dihydro-2(1H)-isoquinolinecarboxylate (2.03 g), N-{[(phenylmethyl)oxy]carbonyl}-L-methionine (2.55 g), HATU (3.42 g) and DIPEA (1.71 ml) were stirred together in DCM (20 ml) at ambient temperature. After 64 h the volatiles were removed and the residue partitioned between water and chloroform. The chloroform layer was washed with 0.5N aqueous HCl (×2), saturated aqueous sodium bicarbonate (×2) and brine and then passed through a hydrophobic frit. This solution was loaded directly onto 2×90 g Biotage™ silica columns and eluted with 1:1 ethyl acetate:cyclohexane, furnishing the title compound as a yellow oil (4.3 g).

Mass spectrum: Found: MH⁺ 514

H.p.l.c. Rₜ 3.52 min

### Intermediate 2

### 1,1-Dimethylethyl 6-[(3S)-2-oxo-3-({[(phenylmethyl)oxy]carbonyl}amino)-1-pyrrolidinyl]-3,4-dihydro-2(1H)-isoquinolinecarboxylate

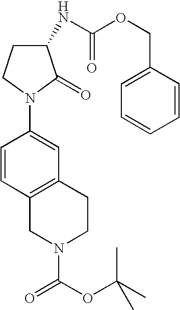

To a solution of 1,1-dimethylethyl 6-[(N-{[(phenylmethyl)oxy]carbonyl}-L-methionyl)amino]-3,4-dihydro-2(1H)-isoquinolinecarboxylate (Intermediate 1) (4.3 g) in dry MeCN (100 ml) under an atmosphere of nitrogen was added methyl iodide (7.8 ml) and the solution stirred for 14 h at ambient temperature. All volatiles were removed and the solution re-dissolved in 100 ml of MeCN under an atmosphere of nitrogen, to which was added caesium carbonate (2.86 g) and the temperature increased to 60° C. for 2 h. After cooling, all volatiles were removed and the residue partitioned between water and chloroform and passed though a hydrophobic frit. The chloroform solution was loaded onto a 330 g Biotage™ silica cartridge and eluted with 1:1 cyclohexane:ethyl acetate, affording the title compound as a white solid (2.68 g).

Mass spectrum: Found: MH⁺ 466

H.p.l.c. Rₜ 3.45 min

### Intermediate 3

### 1,1-Dimethylethyl 6-[(3S)-3-amino-2-oxo-1-pyrrolidinyl]-3,4-dihydro-2(1H)-isoquinolinecarboxylate

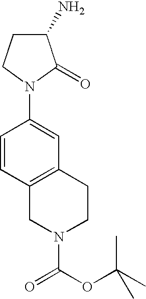

A solution of 1,1-dimethylethyl 6-[(3S)-2-oxo-3-({[(phenylmethyl)oxy]carbonyl}amino)-1-pyrrolidinyl]-3,4-dihydro-2(1H)-isoquinolinecarboxylate (Intermediate 2) (1.0 g) in EtOH (50 ml) was subjected to hydrogenolysis at atmospheric pressure and ambient temperature over 10% Palladium on carbon (Degussa E101 NE/W, 0.1 g), furnishing the title compound (0.595 g) as a white solid after purification on a 50 g silica SPE cartridge eluted with 1:1 cyclohexane:ethyl acetate, then 100:10:1 chlororform:MeOH:0.88 ammonia.

Mass spectrum: Found: MH⁺ 332

H.p.l.c. Rₜ 2.26 min

### Intermediate 4

### 6-Bromo-1-[tris(1-methylethyl)silyl]-1H-indole

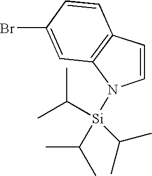

A solution of 6-bromo-1H-indole (2.0 g) in dry THF (20 ml) at 0° C. was treated with sodium hydride (60% dispersion in mineral oil) (0.48 g), in portions, stirring for 30 min. Chloro[tris(1-methylethyl)]silane was added to the reaction, in a dropwise manner, allowing the reaction to warm up to ambient temperature and stir for 18 h. The reaction was concentrated under reduced pressure, then the residue partitioned between DCM and saturated aqueous sodium bicarbonate solution. The organic layer was dried through a hydrophobic frit and re-concentrated to a small volume and loaded onto a pre-conditioned silica SPE cartridge (150 ml/70 g) eluted with ethyl acetate:cyclohexane (0-2%), affording the title compound (3.51 g) as colourless oil.

Mass spectrum: Found: MH⁺ 352

H.p.l.c. Rₜ 4.51 min

### Intermediate 5

### 3-Chloro-1-[tris(1-methylethyl)silyl]-1H-indole-6-sulfonyl chloride

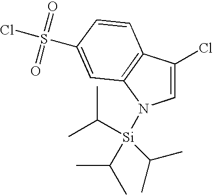

A solution of 6-bromo-1-[tris(1-methylethyl)silyl]-1H-indole (Intermediate 4) (2.30 g) in dry THF (45 ml) at −78° C. was treated with n-butyllithium (1.6M in hexanes) (4.37 ml) in a dropwise manner. The mixture was allowed to stir for 1 h and then poured into a stirred solution of sulphuryl chloride (1.22 ml) in dry cyclohexane (45 ml) at 0° C. The reaction was allowed to warm up to ambient temperature and stir for 2.5 h, then quenched with water (25 ml) stirring for 30 min. The organic layer was passed through a hydrophobic frit and concentrated under reduced pressure. The crude material was dissolved in cyclohexane and loaded onto a pre-conditioned silica phase SPE (150 ml/70 g) eluted with cyclohexane:ethyl acetate (0-10%), affording the title compound (1.18 g) as a beige gum. LCMS data on dimethylamine quench of title compound

Mass spectrum: Found: MH⁺ 415

H.p.l.c. Rₜ 4.22 min

### Intermediate 6

### 1-[Tris(1-methylethyl)silyl]-1H-indole-6-sulfonyl chloride

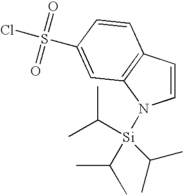

A solution of 6-bromo-1-[tris(1-methylethyl)silyl]-1H-indole (Intermediate 4) (2.64 g) in dry THF (70 ml) at −100° C. was treated with n-butyllithium (1.6M in hexanes) (4.91 ml) in a dropwise manner. The mixture was allowed to stir for 10 min and then sulphur dioxide was condensed into the reaction over 5 min. The reaction was allowed to warm up to ambient temperature and stirred for 3 h, then concentrated under reduced pressure. The oily residue was dissolved in dry cyclohexane (80 ml), cooled to 0° C. then treated with sulphuryl chloride (0.66 ml) stirring for 18 h. The reaction was washed with water (40 ml) and passed through a hydrophobic frit and concentrated under reduced pressure. The residue was dissolved in DCM and loaded onto a pre-conditioned silica Companion™ cartridge (80 g) and eluted with cyclohexane:ethyl acetate (0-10%), which gave the title compound (0.60 g), as an orange solid.

LCMS data on dimethylamine quench of title compound

Mass spectrum: Found: MH⁺ 381

H.p.l.c. Rₜ 4.01 min

### Intermediate 7

### 1,1-Dimethylethyl 6-((3S)-3-{[(6-chloro-2-naphthalenyl)sulfonyl]amino}-2-oxo-1-pyrrolidinyl)-3,4-dihydro-2(1H)-isoquinolinecarboxylate

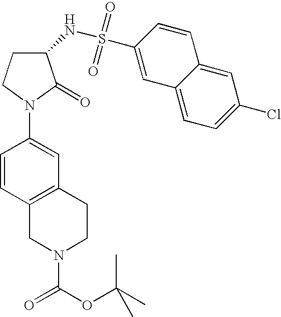

To a solution of 1,1-dimethylethyl 6-[(3S)-3-amino-2-oxo-1-pyrrolidinyl]-3,4-dihydro-2(1H)-isoquinolinecarboxylate (Intermediate 3) (0.148 g) in MeCN (7.4 ml) under nitrogen was added pyridine (0.108 ml) and 6-chloro-2-naphthalenesulfonyl chloride (0.122 g) and the reaction stirred for 14 h at ambient temperature. All volatiles were removed and the residue taken up in chloroform (15 ml) and washed with 0.5N aqueous HCl and saturated aqueous sodium bicarbonate, before separation via a hydrophobic frit. This was purified on a 20 g silica SPE cartridge eluted with a gradient of 1:2 to 1:1 cyclohexane:ethyl acetate, affording the title compound (0.195 g) as a white foam.

Mass spectrum: Found: MH⁺ 556

H.p.l.c. Rₜ 3.71 min

### Intermediate 8

### 1,1-Dimethylethyl 6-[(3S)-3-({[(E)-2-(5-chloro-2-thienyl)ethenyl]sulfonyl}amino)-2-oxo-1-pyrrolidinyl]-3,4-dihydro-2(1H)-isoquinolinecarboxylate

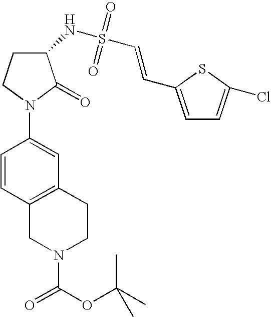

The title compound was prepared using 1,1-dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing (E)-2-(5-chloro-2-thienyl)ethenesulfonyl chloride.

Mass spectrum: Found: MH⁺ 538

H.p.l.c. Rₜ 3.59 min

### Intermediate 9

### 1,1-Dimethylethyl 6-((3S)-3-{[(6-chloro-1-benzothien-2-yl)sulfonyl]amino}-2-oxo-1-pyrrolidinyl)-3,4-dihydro-2(1H)-isoquinolinecarboxylate

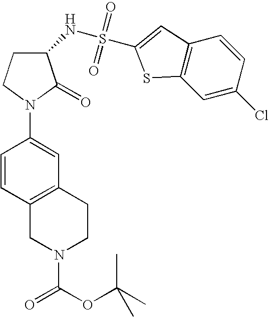

The title compound was prepared using 1,1-dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing 6-chloro-1-benzothiophene-2-sulfonyl chloride.

Mass spectrum: Found: MH⁺ 562

H.p.l.c. Rₜ 3.73 min

### Intermediate 10

### 1,1-Dimethylethyl 6-[(3S)-3-[({3-chloro-1-[tris(1-methylethyl)silyl]-1H-indol-6-yl]sulfonyl)amino]-2-oxo-1-pyrrolidinyl}-3,4-dihydro-2(1H)-isoquinolinecarboxylate

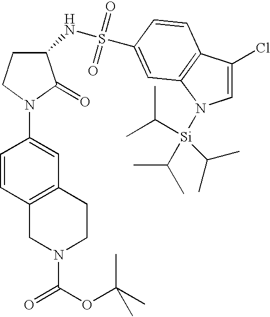

The title compound and a quantity of the N-desilyl analogue were prepared using 11-Dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing 3-chloro-1-[tris(1-methylethyl)silyl]-1H-indole-6-sulfonyl chloride.

Mass spectrum: Found: MH⁺ 701

H.p.l.c. Rₜ 4.33 min

### N-desilyl analogue; 1,1-Dimethylethyl 6-((3S)-3-{[(3-chloro-1H-indol-6-yl)sulfonyl]amino}-2-oxo-1-pyrrolidinyl)-3,4-dihydro-2(1H)-isoquinolinecarboxylate

Mass spectrum: Found: MH⁺ 545

H.p.l.c. Rₜ 3.51 min

### Intermediate 11

### 1,1-Dimethylethyl 6-{(3S)-2-oxo-3-[({1-[tris(1-methylethyl)silyl]-1H-indol-6-yl}sulfonyl)amino]-1-pyrrolidinyl}-3,4-dihydro-2(1H)-isoquinolinecarboxylate

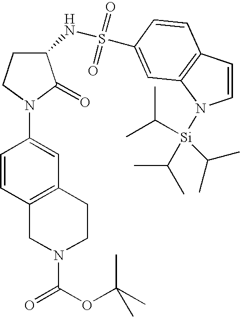

The title compound was prepared using 1,1-dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing 1-[tris(1-methylethyl)silyl]-1H-indole-6-sulfonyl chloride.

Mass spectrum: Found: MH⁺ 667

H.p.l.c. Rₜ 4.18 min

### Intermediate 12

### 1,1-Dimethylethyl 6-((3S)-3-{[(6-chloro-1-{[(1,1-dimethylethyl)oxy]carbonyl}-1H-indol-2-yl)sulfonyl]amino}-2-oxo-1-pyrrolidinyl)-3,4-dihydro-2(1H)-isoquinolinecarboxylate

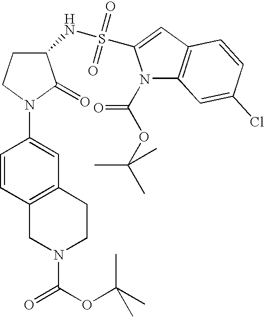

The title compound was prepared using 1,1-dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing 1,1-dimethylethyl 6-chloro-2-(chlorosulfonyl)-1H-indole-1-carboxylate.

Mass spectrum: Found: MH⁺ 645

H.p.l.c. Rₜ 4.04 min

### Intermediate 13

### 1,1-Dimethylethyl ((1S)-3-hydroxy-1-{[(2-methyl-1,2,3,4-tetrahydro-6-isoquinolinyl)amino]carbonyl}propyl)carbamate

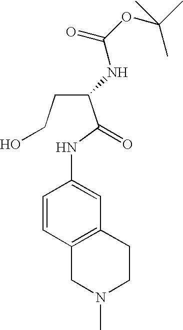

A solution of trimethylaluminium (2M in heptane, 1.4 ml) was added dropwise to a solution of 6-amino-2-methyl-1,2,3,4-tetrahydro-isoquinoline (0.4 g) in DCM (15 ml) at ambient temperature under nitrogen. After reaction for 30 min, 1,1-dimethylethyl[(3S)-2-oxotetrahydro-3-furanyl]carbamate (0.496 g) was added in a further 15 ml of DCM and the reaction stirred at ambient temperature for 14 h. The solution was quenched with 50 ml of 10% aqueous sodium potassium tartrate and the DCM layer passed through a hydrophobic frit, followed by a second DCM extraction. The volume was reduced and the solution applied to a 20 g silica SPE cartridge, eluted with a gradient of cyclohexane:ethyl acetate (1:1 to neat), then 10%-20% MeOH in chloroform (+0.5% 0.88 ammonia), affording the title compound (0.147 g) as a colourless oil.

Mass spectrum: Found: MH⁺ 364

H.p.l.c. Rₜ 1.76 min

### Intermediate 14

### 1,1-Dimethylethyl[(3S)-1-(2-methyl-1,2,3,4-tetrahydro-6-isoquinolinyl)-2-oxo-3-pyrrolidinyl]carbamate

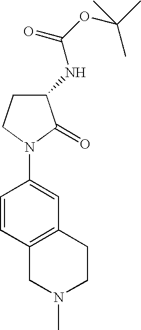

To a solution of 1,1-dimethylethyl ((1S)-3-hydroxy-1-{[(2-methyl-1,2,3,4-tetrahydro-6-isoquinolinyl)amino]carbonyl}propyl)carbamate (Intermediate 13) (0.13 g) in dry THF (20 ml) were added di-tert-butyl azodicarboxylate (0.124 g) and tri-n-butylphosphine (0.13 ml) and the mixture stirred under nitrogen at ambient temperature for 14 h. After evaporation, the residue was purified on a 20 g silica SPE cartridge, eluted with a gradient of cyclohexane:ethyl acetate (1:1 to neat), then 10%-20% MeOH in chloroform (+0.5% 0.88 ammonia), affording 0.227 g of a mixture of the title compound and tri-n-butylphosphine oxide.

Mass spectrum: Found: MH⁺ 346

H.p.l.c. Rₜ 1.90 min

### Intermediate 15

### (3S)-3-Amino-1-(2-methyl-1,2,3,4-tetrahydro-6-isoquinolinyl)-2-pyrrolidinone

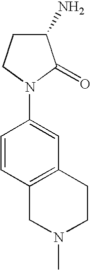

A solution of the impure 1,1-dimethylethyl[(3S)-1-(2-methyl-1,2,3,4-tetrahydro-6-isoquinolinyl)-2-oxo-3-pyrrolidinyl]carbamate (Intermediate 14) (0.227 g), in MeOH (10 ml) which had previously been reacted with acetyl chloride (0.5 ml) to produce hydrogen chloride in situ, was stirred for 14 h. The volatiles were evaporated and the residue purified on a 10 g SCX SPE cartridge, conditioned, loaded and washed with MeOH, then eluted with 10% 0.88 ammonia in MeOH, affording the title compound (0.07 g) as a colourless oil.

Mass spectrum: Found: MH⁺ 246

H.p.l.c. Rₜ 0.22 min

### Intermediate 16

### 1,1-Dimethylethyl 6-((3S)-3-{[(5-chloro-1-benzothien-2-yl)sulfonyl]amino}-2-oxo-1-pyrrolidinyl)-3,4-dihydro-2(1H)-isoquinolinecarboxylate

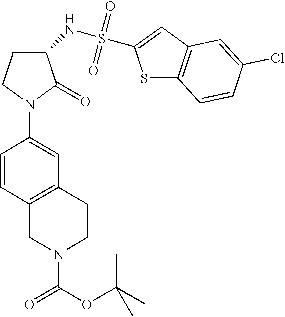

The title compound was prepared using 1,1-dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing 5-chloro-1-benzothiophene-2-sulfonyl chloride.

Mass spectrum: Found: MH⁺ 562

H.p.l.c. Rₜ 3.79 min

### Intermediate 17

### 1,1-Dimethylethyl 6-((3S)-3-{[(5′-chloro-2,2′-bithien-5-yl)sulfonyl]-amino}-2-oxo-1-pyrrolidinyl)-3,4-dihydro-2(1H)-isoquinolinecarboxylate

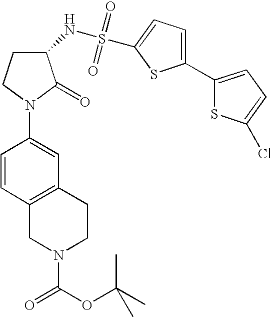

The title compound was prepared using 1,1-dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing 5′-chloro-2,2′-bithiophene-5-sulfonyl chloride.

Mass spectrum: Found: MH⁺ 594

H.p.l.c. Rₜ 3.95 min

### Intermediate 18

### 1,1-Dimethylethyl 7-[(3S)-3-amino-2-oxo-1-pyrrolidinyl]-3,4-dihydro-2(1H)-isoquinolinecarboxylate

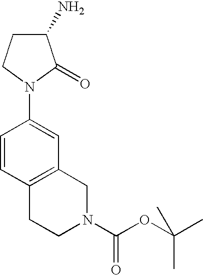

The title compound was prepared using 1,1-dimethylethyl 7-amino-3,4-dihydro-2(1H)-isoquinolinecarboxylate and the procedures described for Intermediates 1, 2 and 3.

Mass spectrum: Found: MH⁺ 332

H.p.l.c. Rₜ 2.28 min

### Intermediate 19

### 1,1-Dimethylethyl 7-((3S)-3-{[(6-chloro-2-naphthalenyl)sulfonyl]amino}-2-oxo-1-pyrrolidinyl)-3,4-dihydro-2(1H)-isoquinolinecarboxylate

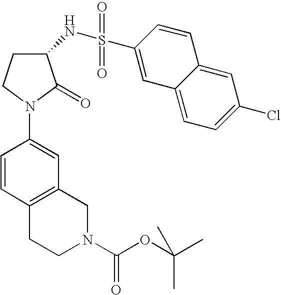

The title compound was prepared using 1,1-dimethylethyl 7-[(3S)-3-amino-2-oxo-1-pyrrolidinyl]-3,4-dihydro-2(1H)-isoquinolinecarboxylate (Intermediate 18) and the procedure described for Intermediate 7 employing 6-chloro-2-naphthalenesulfonyl chloride.

Mass spectrum: Found: MH⁺ 556

H.p.l.c. Rₜ 3.79 min

### Intermediate 20

### 1,1-Dimethylethyl 7-[(3S)-3-({[2-(5-chloro-2-thienyl)ethyl]sulfonyl}amino)-2-oxo-1-pyrrolidinyl]-3,4-dihydro-2(1H)-isoquinolinecarboxylate

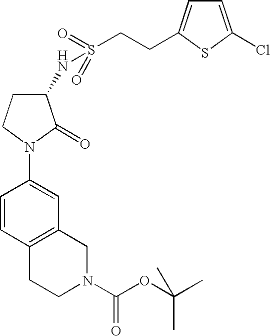

The title compound was prepared using 1,1-dimethylethyl 7-[(3S)-3-amino-2-oxo-1-pyrrolidinyl]-3,4-dihydro-2(1H)-isoquinolinecarboxylate (Intermediate 18) and the procedure described for Intermediate 7 employing 2-(5-chloro-2-thienyl)ethanesulfonyl chloride.

Mass spectrum: Found: MH⁺ 540

H.p.l.c. Rₜ 3.71 min

### Intermediate 21

### 1,1-Dimethylethyl 7-[(3S)-3-({[(E)-2-(5-chloro-2-thienyl)ethenyl]sulfonyl}amino)-2-oxo-1-pyrrolidinyl]-3,4-dihydro-2(1H)-isoquinolinecarboxylate

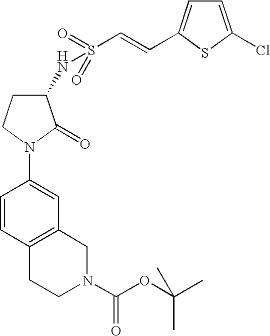

The title compound was prepared using 1,1-dimethylethyl 7-[(3S)-3-amino-2-oxo-1-pyrrolidinyl]-3,4-dihydro-2(1H)-isoquinolinecarboxylate (Intermediate 18) and the procedure described for intermediate 7 employing (E)-2-(5-chloro-2-thienyl)ethenesulfonyl chloride.

Mass spectrum: Found: MH⁺ 538

H.p.l.c. Rₜ 3.68 min

### Intermediate 22

### 1,1-Dimethylethyl 7-{(3S)-3-[({3-chloro-1-[tris(1-methylethyl)silyl]-1H-indol-6-yl}sulfonyl)amino]-2-oxo-1-pyrrolidinyl}-3,4-dihydro-2(1H)-isoquinolinecarboxylate

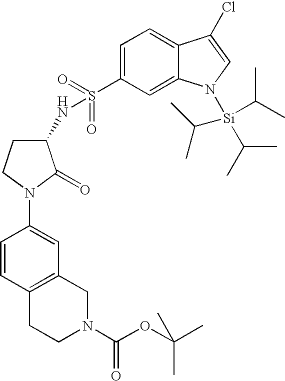

The title compound was prepared using 1,1-dimethylethyl 7-[(3S)-3-amino-2-oxo-1-pyrrolidinyl]-3,4-dihydro-2(1H)-isoquinolinecarboxylate (Intermediate 18) and the procedure described for intermediate 7 employing 3-chloro-1-[tris(1-methylethyl)silyl]-1H-indole-6-sulfonyl chloride.

Mass spectrum: Found: MH⁺ 701

H.p.l.c. Rₜ 4.43 min

### Intermediate 23

### 1,1-Dimethylethyl 7-{(3S)-2-oxo-3-[({1-[tris(1-methylethyl)silyl]-1H-indol-6-yl}sulfonyl)amino]-1-pyrrolidinyl}-3,4-dihydro-2(1H)-isoquinolinecarboxylate

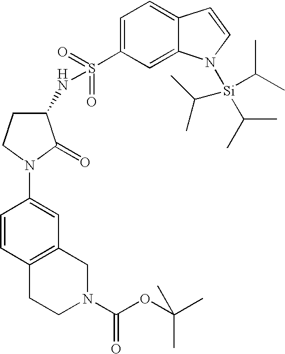

The title compound was prepared using 1,1-dimethylethyl 7-[(3S)-3-amino-2-oxo-1-pyrrolidinyl]-3,4-dihydro-2(1H)-isoquinolinecarboxylate (Intermediate 18) and the procedure described for intermediate 7 employing 1-[tris(1-methylethyl)silyl]-1H-indole-6-sulfonyl chloride.

Mass spectrum: Found: MH⁺ 667

H.p.l.c. Rₜ 4.29 min

### Intermediate 24

### 1,1-Dimethylethyl 6-[(3R)-3-amino-2-oxo-1-pyrrolidinyl]-3,4-dihydro-2(1H)-isoquinolinecarboxylate

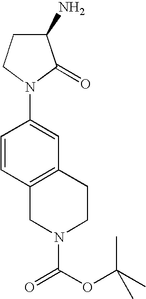

The title compound was prepared using 1,1-dimethylethyl 6-amino-3,4-dihydro-2(1H)-isoquinolinecarboxylate and the procedures described in Intermediates 1, 2 and 3 employing N-{[(phenylmethyl)oxy]carbonyl}-D-methionine in the first step.

Mass spectrum: Found: MH⁺ 332

H.p.l.c. Rₜ 2.35 min

### Intermediate 25

### 1,1-Dimethylethyl 6-((3R)-3-{[(6-chloro-2-naphthalenyl)sulfonyl]amino}-2-oxo-1-pyrrolidinyl)-3,4-dihydro-2(1H)-isoquinolinecarboxylate

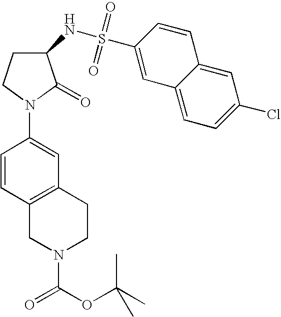

The title compound was prepared using 1,1-dimethylethyl 6-[(3R)-3-amino-2-oxo-1-pyrrolidinyl]-3,4-dihydro-2(1H)-isoquinolinecarboxylate (Intermediate 24) and the procedure described for intermediate 7.

Mass spectrum: Found: MH⁺ 556

H.p.l.c. Rₜ 3.79 min

### Intermediate 26

### Methyl[2-(2,3-difluorophenyl)ethyl]-carbamate

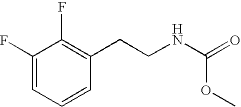

Methyl chloroformate (3.54 ml) was added over five minutes to a solution of [2-(2,3-difluorophenyl)ethyl]amine (6.0 g) in DCM (200 ml) containing pyridine (9.26 ml) cooled to 0° C. under nitrogen. After 1 h the solution was extracted with 200 ml of 1M aqueous HCl (×2), 200 ml of brine and passed through a hydrophobic frit. The solution was reduced in volume and applied directly to a 330 g Companion™ Silica cartridge eluted with a gradient of 30-60% ethyl acetate in cyclohexane. The product fractions were evaporated to afford the title compound as a white solid (6.94 g).

Mass spectrum: Found: MH⁺ 216

H.p.l.c. Rₜ 2.72 min

### Intermediate 27

### 5,6-Difluoro-3,4-dihydro-1(2H)-isoquinolinone

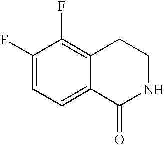

Phosphorus pentoxide (7.0 g) was added portion-wise to polyphosphoric acid with mechanical stirring at 130° C. On completion of the addition, the temperature was raised to 150° C. for 30 min before Intermediate 26 (2.0 g) was added and the temperature maintained for a further 30 min. The solution was then poured onto 200 ml of ice and this mixture subsequently extracted with 3×75 ml portions of chloroform. The combined organic extracts were washed with water and brine, then passed through a hydrophobic frit and evaporated. The title compound (0.298 g) was isolated as a white solid after purification on a 40 g Companion™ Silica cartridge eluted with a gradient of 50-100% ethyl acetate: cyclohexane.

Mass spectrum: Found: MH⁺ 184

H.p.l.c. Rₜ 2.31 min

### Intermediate 28

### 6-Amino-5-fluoro-3,4-dihydro-1(2H)-isoquinolinone

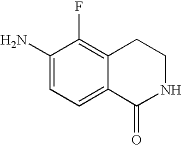

A suspension of 5,6-difluoro-3,4-dihydro-1(2H)-isoquinolinone (Intermediate 27) (0.513 g) in 2M ammonia in MeOH (5 ml) and 35% aqueous ammonia (10 ml) was stirred and heated at 160° C. in an autoclave for 22 h. On cooling, the volatiles were removed and the residue purified on a 20 g SCX SPE column, which was washed with MeOH and eluted with 2M ammonia in MeOH, affording the title compound (0.40 g) as a white solid.

Mass spectrum: Found: MH⁺ 181

H.p.l.c. Rₜ 1.71 min

### Intermediate 29

### 5-Fluoro-1,2,3,4-tetrahydro-6-isoquinolinamine

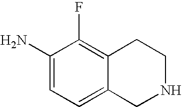

To a solution of 6-amino-5-fluoro-3,4-dihydro-1(2H)-isoquinolinone (Intermediate 28) (0.421 g) in THF (10 ml) under nitrogen at room temperature, was added 16.5 ml of a 1M solution of borane in THF. The temperature was increased to give reflux and maintained for 3 h. The solution was cooled and 20 ml of MeOH added and stirred for 20 min at ambient temperature. The volatiles were removed and the residue dissolved in MeOH (20 ml), to which 20 ml of 6M aqueous HCl was added; then the mixture was heated under reflux for 2 h, before cooling to ambient temperature. The pH of this solution was raised by the addition of excess 10N aqueous sodium hydroxide and three 40 ml DCM extractions were made. These combined extracts were washed with brine, passed through a hydrophobic frit and evaporated. The title compound (0.332 g) was isolated from a 20 g SCX SPE column, which was washed with MeOH and eluted with 2M ammonia in MeOH.

Mass spectrum: Found: MH⁺ 167

H.p.l.c. Rₜ 0.34 min

### Intermediate 30

### 1,1-Dimethylethyl 6-amino-5-fluoro-3,4-dihydro-2(1H)-isoquinolinecarboxylate

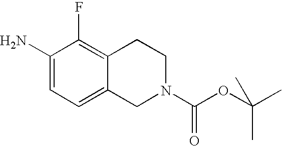

A solution of 5-fluoro-1,2,3,4-tetrahydro-6-isoquinolinamine (Intermediate 29) (0.329 g) in DCM (15 ml) was treated with bis(1,1-dimethylethyl) dicarbonate (0.433 g) at room temperature over 96 h. The solution was washed with water (15 ml), then brine (15 ml) and passed though a hydrophobic frit before partial evaporation. The title compound (0.352 g), a colourless oil, was isolated after purification on a 40 g Companion™ Silica cartridge eluted with a gradient of 5-50% ethyl acetate: cyclohexane.

Mass spectrum: Found: MH⁺ 267

H.p.l.c. Rₜ 3.05 min

### Intermediate 31

### 1,1-Dimethylethyl 6-[(3S)-3-amino-2-oxo-1-pyrrolidinyl]-5-fluoro-3,4-dihydro-2(1H)-isoquinolinecarboxylate

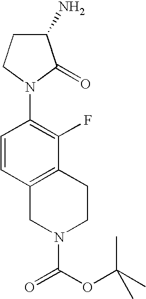

The title compound was prepared using 1,1-dimethylethyl 6-amino-5-fluoro-3,4-dihydro-2(1H)-isoquinolinecarboxylate (Intermediate 30) and the procedures described for Intermediates 1, 2 and 3.

Mass spectrum: Found: MH⁺ 350

H.p.l.c. Rₜ 2.31 min

### Intermediate 32

### 1,1-Dimethylethyl 6-((3S)-3-{[(6-chloro-2-naphthalenyl)sulfonyl]amino}-2-oxo-1-pyrrolidinyl)-5-fluoro-3,4-dihydro-2(1H)-isoquinolinecarboxylate

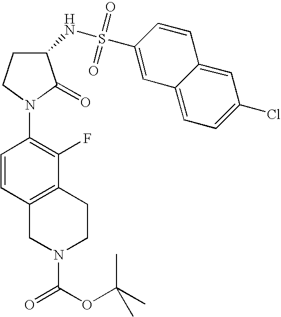

The title compound was prepared using 1,1-dimethylethyl 6-[(3S)-3-amino-2-oxo-1-pyrrolidinyl]-5-fluoro-3,4-dihydro-2(1H)-isoquinolinecarboxylate (Intermediate 31) and the procedure described for Intermediate 7 employing 6-chloro-2-naphthalenesulfonyl chloride.

Mass spectrum: Found: MH⁺ 574

H.p.l.c. Rₜ 3.80 min

### Intermediate 33

### 1,1-Dimethylethyl 6-[(3S)-3-({[(E)-2-(5-chloro-2-thienyl)ethenyl]sulfonyl}amino)-2-oxo-1-pyrrolidinyl]-5-fluoro-3,4-dihydro-2(1H)-isoquinolinecarboxylate

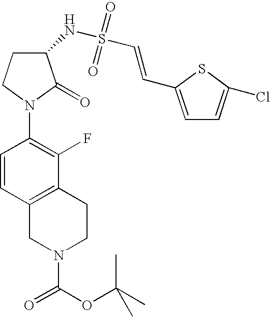

The title compound was prepared using 1,1-dimethylethyl 6-[(3S)-3-amino-2-oxo-1-pyrrolidinyl]-5-fluoro-3,4-dihydro-2(1H)-isoquinolinecarboxylate (Intermediate 31) and the procedure described for Intermediate 7, employing (E)-2-(5-chloro-2-thienyl)ethenesulfonyl chloride.

Mass spectrum: Found: MH⁺ 556

H.p.l.c. Rₜ 3.57 min

### Intermediate 34

### 1,1-Dimethylethyl 6-{(3S)-3-[({3-chloro-1-[tris(1-methylethyl)silyl]-1H-indol-6-yl}sulfonyl)amino]-2-oxo-1-pyrrolidinyl}-5-fluoro-3,4-dihydro-2(1H)-isoquinolinecarboxylate

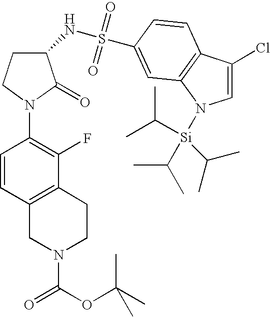

The title compound together with the N-desilyl analogue were prepared using 1,1-dimethylethyl 6-[(3S)-3-amino-2-oxo-1-pyrrolidinyl]-5-fluoro-3,4-dihydro-2(1H)-isoquinolinecarboxylate (Intermediate 31) and the procedure described for Intermediate 7, employing 3-chloro-1-[tris(1-methylethyl)silyl]-1H-indole-6-sulfonyl chloride.

Mass spectrum: Found: MH⁺ 719

H.p.l.c. Rₜ 4.32 min

### N-desilyl analogue; 1,1-dimethylethyl 6-((3S)-3-{[(3-chloro-1H-indol-6-yl)sulfonyl]amino}-2-oxo-1-pyrrolidinyl)-5-fluoro-3,4-dihydro-2(1H)-isoquinolinecarboxylate

Mass spectrum: Found: MH⁺ 563

H.p.l.c. Rₜ 3.56 min

### Intermediate 35

### 2,2,2-Trifluoro-N-[2-(4-fluorophenyl)ethyl]acetamide

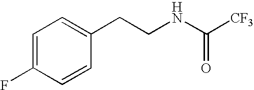

A solution of 4-fluorophenethylamine (4.37 g) and triethylamine (4.38 ml) in anhydrous DCM (35 ml) was stirred, under nitrogen and cooled to −5° C. Trifluoroacetic anhydride (4.37 ml) was added drop wise over 40 min, maintaining the temperature between −5° C. and 0° C. The pale yellow solution was stirred in the cooling bath for 1 h during which time the temperature reached 8° C. The reaction mixture was evaporated to dryness and the residual oil was treated with MeOH (50 ml) and evaporated to dryness. The yellow oil was treated with water (100 ml) and the suspension was stirred at room temperature for 1 h. The solid was collected by filtration, washed well with water and dried to give the title compound as a colourless solid (7.0 g).

Mass spectrum: Found MH⁺ 234

H.p.l.c. Rₜ 2.95 min

### Intermediate 36

### 7-Fluoro-2-(trifluoroacetyl)-1,2,3,4-tetrahydroisoquinoline

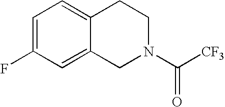

2,2,2-Trifluoro-N-[2-(4-fluorophenyl)ethyl]acetamide (Intermediate 35) (4.84 g) was added to a mixture of concentrated sulfuric acid (10 ml) and glacial acetic acid (15 ml) stirring at room temperature. Paraformaldehyde (1.02 g) was added and the cloudy mixture was stirred, under nitrogen, at room temperature for 20 h giving a yellow solution. This solution was poured into cold water (400 ml) and extracted with ethyl acetate (2×150 ml). The organic extracts were combined, washed with saturated aqueous sodium hydrogen carbonate solution (2×150 ml), brine (2×100 ml), dried (anhydrous sodium sulfate), filtered and evaporated to dryness. The residue was purified on a 330 g silica column eluted with DCM to give the title compound as a colourless oil (3.58 g).

Mass spectrum: Found MH⁺ 248

H.p.l.c. Rₜ 3.15 min

### Intermediate 37

### 7-Fluoro-6-nitro-2-(trifluoroacetyl)-1,2,3,4-tetrahydroisoquinoline

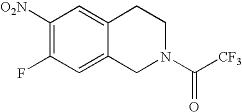

7-Fluoro-2-(trifluoroacetyl)-1,2,3,4-tetrahydroisoquinoline (Intermediate 36) (5.15 g) was added slowly to ice-cold concentrated sulfuric acid (21 ml). The yellow solution was cooled to 0° C. and an ice cold solution of potassium nitrate (2.10 g) in concentrated sulfuric acid (30 ml) was added over 75 min keeping the temperature between 0 and 2° C. The reaction mixture was then stirred at 4° C. for 45 min, poured into ice/water (1000 ml) and extracted with ethyl acetate (2×350 ml). The organic extracts were combined, washed with water (2×400 ml), brine (400 ml), dried (anhydrous sodium sulfate), filtered and evaporated to dryness. The residue was dissolved, with warming, in DCM (25 ml), cooled to room temperature and diluted with cyclohexane (15 ml) and the resulting precipitate was collected by filtration, washed with cyclohexane and dried in vacuo to give the title compound as a light brown solid. (2.71 g).

Mass spectrum: Found MH⁺ 291

H.p.l.c. Rₜ 3.10 min

### Intermediate 38

### 7-Fluoro-6-nitro-1,2,3,4-tetrahydroisoquinoline hydrochloride

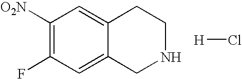

A suspension of 7-fluoro-6-nitro-2-(trifluoroacetyl)-1,2,3,4-tetrahydroisoquinoline (Intermediate 37) (3.26 g) in MeOH (30 ml) and 2N aqueous hydrochloric acid (30 ml) was heated at reflux, under nitrogen, for 11 h and evaporated to dryness. The residue was triturated with diethyl ether and the pale orange title compound was collected by filtration and dried (2.48 g).

¹H NMR (DMSO-d₆) δ: 3.08 (2H, m), 3.38 (2H, m), 4.35 (2H, s), 7.54 (1H, d), 8.11 (1H, d), 8.72 (1H, brs)

### Intermediate 39

### 1,1-Dimethylethyl 7-fluoro-6-nitro-3,4-dihydro-2(1H)-isoquinolinecarboxylate

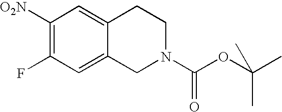

A solution of 7-fluoro-6-nitro-1,2,3,4-tetrahydroisoquinoline hydrochloride (Intermediate 38) (2.48 g) in dioxane (50 ml) and water (7 ml) was treated with triethylamine (2.97 ml) and bis(1,1-dimethylethyl) dicarbonate (2.33 g) and stirred at room temperature, under nitrogen, for 22 h. The solution was evaporated to remove volatiles and the slurry was partitioned between saturated aqueous sodium hydrogen carbonate solution (40 ml) and DCM (2×40 ml). The organic extracts were combined, washed with brine, passed through a hydrophobic frit and evaporated to near dryness. The slurry was treated with cyclohexane (75 ml) and the title compound was collected by filtration as a light brown solid (2.40 g).

Mass spectrum: Found MH⁺ 295

H.p.l.c. Rₜ 3.37 min

### Intermediate 40

### 1,1-Dimethylethyl 6-amino-7-fluoro-3,4-dihydro-2(1H)-isoquinolinecarboxylate

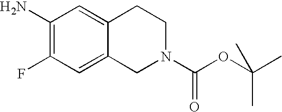

A suspension of 1,1-dimethylethyl 7-fluoro-6-nitro-3,4-dihydro-2(1H)-isoquinolinecarboxylate (Intermediate 39) (2.80 g) in EtOH (200 ml) was hydrogenated at atmospheric pressure in the presence of 10% palladium on charcoal (wet) (800 mg) for 3 h, filtered and evaporated to a dark oil. The oil was azeotroped with DCM to give the title compound as a dark oil (2.38 g).

Mass spectrum: Found MH⁺ 267

H.p.l.c. Rₜ 3.00 min

### Intermediate 41

### 1,1-Dimethylethyl 6-[(3S)-3-amino-2-oxo-1-pyrrolidinyl]-7-fluoro-3,4-dihydro-2(1H)-isoquinolinecarboxylate

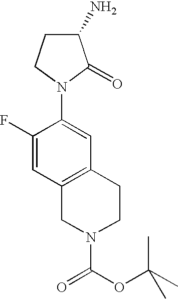

The title compound was prepared using 1,1-dimethylethyl 6-amino-7-fluoro-3,4-dihydro-2(1H)-isoquinolinecarboxylate (Intermediate 40) and the procedures described for Intermediates 1, 2 and 3.

Mass spectrum: Found MH⁺ 350

H.p.l.c. Rₜ 2.06 min

### Intermediate 42

### 1,1-Dimethylethyl 6-(3-{[(6-chloro-2-naphthalenyl)sulfonyl]amino}-2-oxo-1-pyrrolidinyl)-7-fluoro-3,4-dihydro-2(1H)-isoquinolinecarboxylate

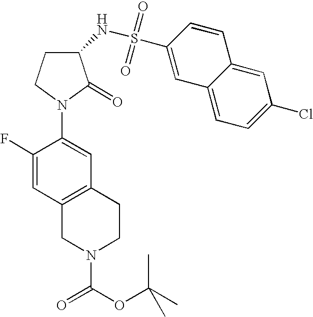

The title compound was prepared using 1,1-dimethylethyl 6-[(3S)-3-amino-2-oxo-1-pyrrolidinyl]-7-fluoro-3,4-dihydro-2(1H)-isoquinolinecarboxylate (Intermediate 41) and the procedure described for Intermediate 7, employing 6-chloro-2-naphthalenesulfonyl chloride.

Mass spectrum: Found MH⁺ 574.

H.p.l.c. Rₜ 3.76 min

### Intermediate 43

### 1,1-Dimethylethyl 6-((3S)-3-{[(6-chloro-1-benzothien-2-yl)sulfonyl]amino}-2-oxo-1-pyrrolidinyl)-7-fluoro-3,4-dihydro-2(1H)-isoquinolinecarboxylate

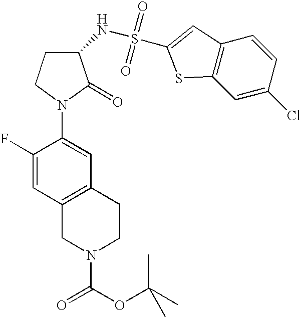

The title compound was prepared using 1,1-dimethylethyl 6-[(3S)-3-amino-2-oxo-1-pyrrolidinyl]-7-fluoro-3,4-dihydro-2(1H)-isoquinolinecarboxylate (Intermediate 41) and the procedure described for Intermediate 7, employing 6-chloro-1-benzothiophene-2-sulfonyl chloride.

Mass spectrum: Found MH⁺ 578

H.p.l.c. Rₜ 3.71 min

### Intermediate 44

### 1,1-Dimethylethyl 6-[(3S)-3-({[(E)-2-(5-chloro-2-thienyl)ethenyl]sulfonyl}amino)-2-oxo-1-pyrrolidinyl]-7-fluoro-3,4-dihydro-2(1H)-isoquinoline carboxylate

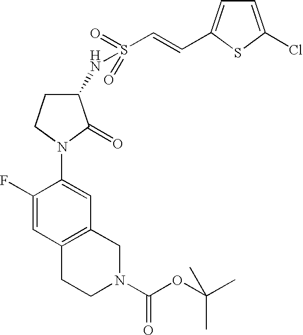

The title compound was prepared using 1,1-dimethylethyl 6-[(3S)-3-amino-2-oxo-1-pyrrolidinyl]-7-fluoro-3,4-dihydro-2(1H)-isoquinolinecarboxylate (Intermediate 41) and the procedure described for Intermediate 7, employing (E)-2-(5-chloro-2-thienyl)ethenesulfonyl chloride.

Mass spectrum: Found MH⁺ 554

H.p.l.c. Rₜ 3.55 min

### Intermediate 45

### 1-Dimethylethyl 6-{(3S)-3-[({3-chloro-1-[tris(1-methylethyl)silyl]-1H-indol-6-yl}sulfonyl)amino]-2-oxo-1-pyrrolidinyl}-7-fluoro-3,4-dihydro-2(1H)-isoquinolinecarboxylate

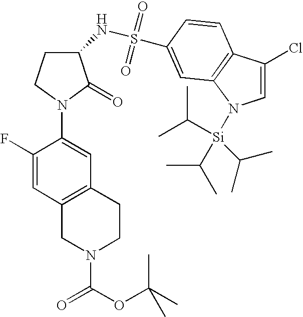

The title compound was prepared using 1,1-dimethylethyl 6-[(3S)-3-amino-2-oxo-1-pyrrolidinyl]-7-fluoro-3,4-dihydro-2(1H)-isoquinolinecarboxylate (Intermediate 41) and the procedure described for Intermediate 7, employing Intermediate 5.

Mass spectrum: Found MH⁺ 719

H.p.l.c. Rₜ 4.33 min

### Intermediate 46

### 1,1-Dimethylethyl 7-chloro-6-nitro-3,4-dihydro-2(1H)-isoquinolinecarboxylate

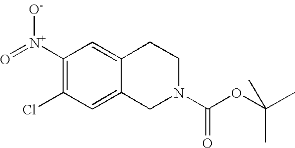

The title compound was prepared from [2-(4-chlorophenyl)ethyl]amine using the procedures described for Intermediates 35, 36, 37, 38 and 39.

H.p.l.c. Rₜ 3.52 min

¹H NMR (CDCl₃) δ: 1.50 (9H, s) 2.88 (2H, t), 3.68 (2H, t), 4.61 (2H, s), 7.31 (1H, brs), 7.72 (1H, brs)

### Intermediate 47

### 1,1-Dimethylethyl 6-amino-7-chloro-3,4-dihydro-2(1H)-isoquinolinecarboxylate

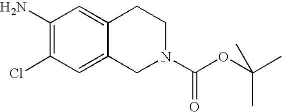

Tin (II) chloride dihydrate (12.62 g) was added portionwise over 35 min to a solution of 1,1-dimethylethyl 7-chloro-6-nitro-3,4-dihydro-2(1H)-isoquinolinecarboxylate (Intermediate 46) (3.5 g) in ethyl acetate (250 ml) at room temperature and the solution was stirred for 24 h under nitrogen at ambient temperature. Saturated aqueous sodium bicarbonate was added and well stirred, before a thick precipitate was filtered and washed with ethyl acetate (100 ml) and water (20 ml). The filtered layers were separated and the aqueous phase was extracted with ethyl acetate (50 ml). The combined organic layers were washed with brine and dried over sodium sulfate. After evaporation, the residue was purified on a 120 g Redisep™ silica cartridge eluted with a gradient of 10-50% ethyl acetate: cyclohexane, affording the title compound (2.59 g) as a colourless oil that solidified on standing.

Mass spectrum: Found MH⁺ 283, 285

H.p.l.c. Rₜ 3.28 min

### Intermediate 48

### 1,1-Dimethylethyl 7-chloro-6-[(3S)-3-({[(9H-fluoren-9-ylmethyl)oxy]carbonyl}amino)-2-oxo-1-pyrrolidinyl]-3,4-dihydro-2(1H)-isoquinolinecarboxylate

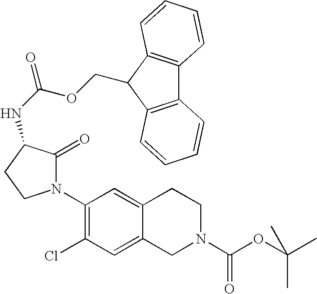

The title compound was prepared from 1,1-dimethylethyl 6-amino-7-chloro-3,4-dihydro-2(1H)-isoquinolinecarboxylate (Intermediate 47) and N-[(9H-fluoren-9-ylmethyl)oxy]carbonyl-L-methionine using the procedures described for Intermediates 1 and 2.

Mass spectrum: Found MH⁺ 588, 590

H.p.l.c. Rₜ 3.77 min

### Intermediate 49

### 1,1-Dimethylethyl 6-[(3S)-3-amino-2-oxo-1-pyrrolidinyl]-7-chloro-3,4-dihydro-2(1H)-isoquinolinecarboxylate

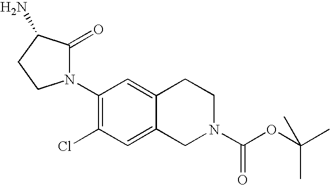

A solution of 1,1-dimethylethyl 7-chloro-6-[(3S)-3-({[(9H-fluoren-9-ylmethyl)oxy]carbonyl}amino)-2-oxo-1-pyrrolidinyl]-3,4-dihydro-2(1H)-isoquinolinecarboxylate (Intermediate 48) (0.4 g) and piperidine (0.75 ml) in anhydrous dimethylformamide was stirred at ambient temperature under nitrogen for 70 min. All volatiles were removed and the title compound (0.206 g) was isolated as a yellow gum from a 10 g silica SPE column, which was eluted with a 0 to 3% gradient of 2M methanolic ammonia in DCM.

Mass spectrum: Found MH⁺ 366, 368

H.p.l.c. Rₜ 2.33 min

### Intermediate 50

### 1,1-Dimethylethyl 7-chloro-6-((3S)-3-{[(6-chloro-2-naphthalenyl)sulfonyl]amino}-2-oxo-1-pyrrolidinyl)-3,4-dihydro-2(1H)-isoquinolinecarboxylate

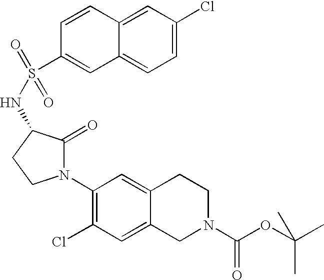

The title compound was prepared using 1,1-dimethylethyl 6-[(3S)-3-amino-2-oxo-1-pyrrolidinyl]-7-chloro-3,4-dihydro-2(1H)-isoquinolinecarboxylate (Intermediate 49) and the procedure described for Intermediate 7, employing 6-chloro-2-naphthalenesulfonyl chloride.

Mass spectrum: Found MH⁺ 590, 592

H.p.l.c. Rₜ 3.76 min

### Intermediate 51

### 1,1-Dimethylethyl 7-chloro-6-[(3S)-3-({[(E)-2-(5-chloro-2-thienyl)ethenyl]sulfonyl}amino)-2-oxo-1-pyrrolidinyl]-3,4-dihydro-2(1H)-isoquinolinecarboxylate

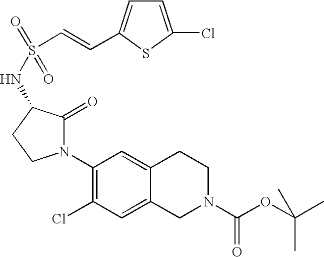

The title compound was prepared using 1,1-dimethylethyl 6-[(3S)-3-amino-2-oxo-1-pyrrolidinyl]-7-chloro-3,4-dihydro-2(1H)-isoquinolinecarboxylate (Intermediate 49) and the procedure described for Intermediate 7, employing (E)-2-(5-chloro-2-thienyl)ethenesulfonyl chloride.

Mass spectrum: Found MH⁺ 572, 574

H.p.l.c. Rₜ 3.63 min

### Intermediate 52

### 1,1-Dimethylethyl 7-chloro-6-{((3S)-3-[({3-chloro-1-[tris(1-methylethyl)silyl]-1H-indol-6-yl}sulfonyl)amino]-2-oxo-1-pyrrolidinyl}-3,4-dihydro-2(1H)-isoquinolinecarboxylate

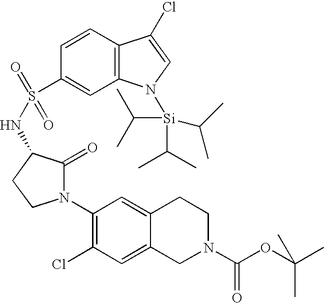

The title compound was prepared using 1,1-dimethylethyl 6-[(3S)-3-amino-2-oxo-1-pyrrolidinyl]-7-chloro-3,4-dihydro-2(1H)-isoquinolinecarboxylate (Intermediate 49) and the procedure described for Intermediate 7, employing 3-chloro-1-[tris(1-methylethyl)silyl]-1H-indole-6-sulfonyl chloride.

Mass spectrum: Found MH⁺ 735, 737

H.p.l.c. Rₜ 4.39 min

### Intermediate 53

### 1,1-Dimethylethyl 6-amino-7-methyl-3,4-dihydro-2(1H)-isoquinolinecarboxylate

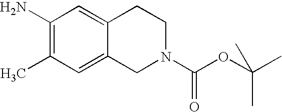

The title compound was prepared from [2-(4-methylphenyl)ethyl]amine using the procedures described for Intermediates 35, 36, 37, 38, 39 and 40.

Mass spectrum: Found MH⁺ 263

H.p.l.c. Rₜ 2.78 min

### Intermediate 54

### 1,1-Dimethylethyl 6-[(3S)-3-amino-2-oxo-1-pyrrolidinyl]-7-methyl-3,4-dihydro-2(1H)-isoquinolinecarboxylate

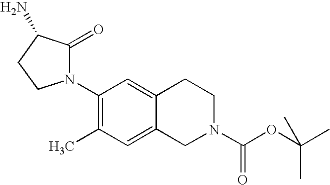

The title compound was prepared using 1,1-dimethylethyl 6-amino-7-methyl-3,4-dihydro-2(1H)-isoquinolinecarboxylate (Intermediate 53) and the procedures described for Intermediates 1, 2 and 3.

Mass spectrum: Found MH⁺ 346

H.p.l.c. Rₜ 2.27 min

### Intermediate 55

### 1,1-Dimethylethyl 6-((3S)-3-{[(6-chloro-2-naphthalenyl)sulfonyl]amino}-2-oxo-1-pyrrolidinyl)-7-methyl-3,4-dihydro-2(1H)-isoquinolinecarboxylate

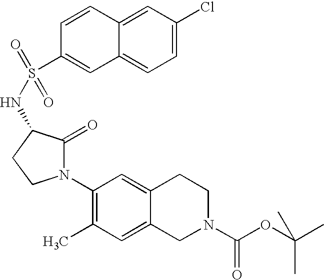

The title compound was prepared using 1,1-dimethylethyl 6-[(3S)-3-amino-2-oxo-1-pyrrolidinyl]-7-methyl-3,4-dihydro-2(1H)-isoquinolinecarboxylate (Intermediate 54) and the procedure described for Intermediate 7, employing 6-chloro-2-naphthalenesulfonyl chloride.

Mass spectrum: Found MH⁺ 570, 572

H.p.l.c. Rₜ 3.70 min

### Intermediate 56

### 1,1-Dimethylethyl 6-[(3S)-3-({[(E)-2-(5-chloro-2-thienyl)ethenyl]sulfonyl}amino)-2-oxo-1-pyrrolidinyl]-7-methyl-3,4-dihydro-2(1H)-isoquinolinecarboxylate

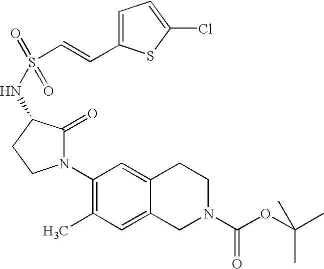

The title compound was prepared using 1,1-dimethylethyl 6-[(3S)-3-amino-2-oxo-1-pyrrolidinyl]-7-methyl-3,4-dihydro-2(1H)-isoquinolinecarboxylate (Intermediate 54) and the procedure described for Intermediate 7, employing (E)-2-(5-chloro-2-thienyl)ethenesulfonyl chloride.

Mass spectrum: Found MH⁺ 552, 554

H.p.l.c. Rₜ 3.57 min

### Intermediate 57

### 1,1-Dimethylethyl 6-{(3S)-3-[({3-chloro-1-tris(1-methylethyl)silyl]-1H-indol-6-yl}sulfonyl)amino]-2-oxo-1-pyrrolidinyl}-7-methyl-3,4-dihydro-2(1H)-isoquinolinecarboxylate

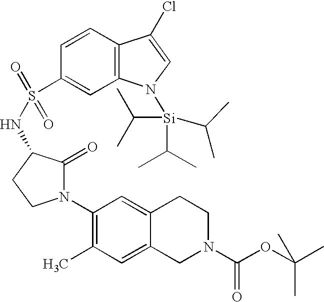

The title compound was prepared using 1,1-dimethylethyl 6-[(3S)-3-amino-2-oxo-1-pyrrolidinyl]-7-methyl-3,4-dihydro-2(1H)-isoquinolinecarboxylate (Intermediate 54) and the procedure described for Intermediate 7, employing 3-chloro-1-[tris(1-methylethyl)silyl]-1H-indole-6-sulfonyl chloride.

Mass spectrum: Found MH⁺ 715, 717

H.p.l.c. Rₜ 4.34 min

### Intermediate 58

### 7-Bromo-6-nitro-2-(trifluoroacetyl)-1,2,3,4-tetrahydroisoquinoline

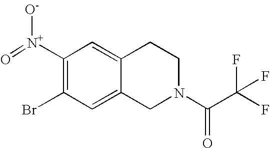

A 2 L flask was charged with concentrated sulfuric acid (300 ml) and the reaction mixture was stirred mechanically and cooled to 0° C. 7-Bromo-2-(trifluoroacetyl)-1,2,3,4-tetrahydroisoquinoline (WO 2004060882) (92.4 g) was added in portions over 45 min. with mechanical stirring and ice-water cooling to 0° C. A previously prepared solution of potassium nitrate (30.4 g, 1 eq.) in concentrated sulfuric acid (460 ml) was added dropwise over 2 h maintaining the internal temperature between 0 and 2° C. The mixture was stirred at 0° C. for a further 30 min then allowed to warm to room temperature and stirred for a further 1 h before being poured into ice-water (8 L). The precipitate was extracted into ethyl acetate (8 L) and the organic phase was washed with saturated aqueous sodium hydrogen carbonate (2 L), dried over sodium sulfate (overnight) filtered and concentrated under reduced pressure to give a yellow solid (105.65 g). This was boiled with DCM (500 ml) and cyclohexane (200 ml) and the hot solution was decanted from insoluble material (24.65 g of a yellow solid) and allowed to cool to room temperature and stand overnight to give further yellow solid (23.15 g) The 2 batches of yellow solid were combined to give the title compound (47.80 g).

Mass spectrum: Found [M-H⁺]⁻ 351, 353

H.p.l.c. Rₜ 3.25 min

### Intermediate 59

### 2-(Trifluoroacetyl)-1,2,3,4-tetrahydro-6-isoquinolinamine

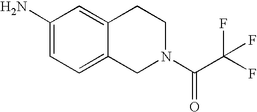

7-Bromo-6-nitro-2-(trifluoroacetyl)-1,2,3,4-tetrahydroisoquinoline (Intermediate 58) (7.06 g) was added to 10% palladium on carbon (Degussa, wetted 1:1 w:w with water, 3.5 g) under nitrogen in a hydrogenation flask. The mixture was hydrogenated in EtOH (150 ml) at 1 atmosphere of hydrogen pressure for 5 h. The mixture was filtered (glass microfibre filters) with suction and the filtrate was evaporated under reduced pressure to give a pink solid; this was suspended in DCM (250 ml) and stirred with saturated aqueous sodium hydrogen carbonate (200 ml). The organic phase was separated (hydrophobic frit) and evaporated under reduced pressure to give the title compound as a pink gum (4.91 g).

Mass spectrum: Found [MH⁺] 245

H.p.l.c. Rₜ 2.19 min

### Intermediate 60

### 6-(2,5-Dimethyl-1H-pyrrol-1-yl)-2-(trifluoroacetyl)-1,2,3,4-tetrahydroisoquinoline

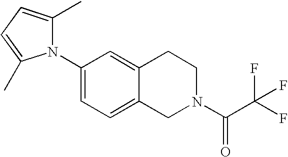

2-(Trifluoroacetyl)-1,2,3,4-tetrahydro-6-isoquinolinamine (Intermediate 59) (4.884 g) was dissolved in toluene (100 ml) and the solution was treated with 2,5-hexanedione (2.306 g). The mixture was heated at reflux under nitrogen in the presence of 4-toluenesulfonic acid hydrate (38 mg) in a Dean-Stark separator for 2.5 h. The initially pale orange solution was allowed to cool to room temperature and left overnight to give a deep purple solution. This was diluted with ethyl acetate (100 ml) and washed with saturated aqueous sodium hydrogen carbonate (100 ml) to ensure removal of the sulfonic acid. The organic phase was separated and washed with water (3×100 ml), dried over sodium sulfate, filtered and evaporated under reduced pressure to give a brown solid which was triturated under cyclohexane to give the title compound as a pale brown solid (5.431 g).

Mass spectrum: Found [MH⁺] 323

H.p.l.c. Rₜ 3.67 min

### Intermediate 61

### 6-(2,5-Dimethyl-1H-pyrrol-1-yl)-1,2,3,4-tetrahydroisoquinoline

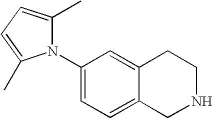

6-(2,5-Dimethyl-1H-pyrrol-1-yl)-2-(trifluoroacetyl)-1,2,3,4-tetrahydroisoquinoline (Intermediate 60) (5.360 g) was stirred in MeOH (135 ml) and water (15 ml) and solid potassium carbonate (12.14 g) was added in one portion. The mixture was heated at reflux under nitrogen for 3 h. The mixture was filtered and the filtrate was evaporated under reduced pressure to give a brown oil plus solid; this mixture was taken up in DCM (200 ml) and washed with saturated aqueous sodium carbonate (200 ml). The organic phase was separated (hydrophobic frit), dried over sodium sulfate and evaporated under reduced pressure to give the title compound as a brown solid (3.474 g)

Mass spectrum: Found [MH⁺] 22

H.p.l.c. Rₜ 2.10 min

### Intermediate 62

### 6-(2,5-Dimethyl-1H-pyrrol-1-yl)-3,4-dihydroisoquinoline

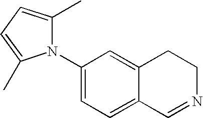

6-(2,5-Dimethyl-1H-pyrrol-1-yl)-1,2,3,4-tetrahydroisoquinoline (Intermediate 61) (4.352 g) was dissolved in DCM (55 ml) and activated manganese dioxide (21.04 g) was added in one portion. The mixture was stirred vigorously at room temperature for 16 h, then filtered. The solids were washed with DCM (200 ml), and the combined filtrate and washings were evaporated under reduced pressure and purified by flash chromatography on silica (80 g Redisep™ cartridge eluted with 30%-100% ethyl acetate in cyclohexane) to give the title compound (2.980 g) as a tan solid.

Mass spectrum: Found [MH⁺] 225

H.p.l.c.Rₜ 2.13 min

### Intermediate 63

### 6-(2,5-Dimethyl-1H-pyrrol-1-yl)-3,4-dihydroisoquinoline:boron trifluoride complex

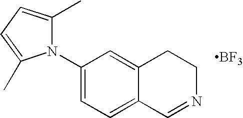

6-(2,5-Dimethyl-1H-pyrrol-1-yl)-3,4-dihydroisoquinoline (Intermediate 62) (1.620 g) was dissolved in dry THF (30 ml) and the solution was stirred under nitrogen at −78° C. Boron trifluoride etherate (2 ml) was added and the mixture was stirred at −78° C. for 5 min then treated with a solution of methylmagnesium bromide (1.4M solution in toluene:THF, 4 ml). The mixture was stirred at −78° C. for 1 h then allowed to warm to room temperature and stirred at room temperature overnight. It was cooled again to −78° C. then quenched by addition of saturated aqueous ammonium chloride (5 ml), allowed to warm to room temperature then diluted with ethyl acetate (200 ml). Sufficient solid sodium carbonate was then added to absorb the free water. The organic phase was separated and evaporated under reduced pressure. The crude product was purified by flash chromatography on silica (40 g Redisep™ cartridge eluted with 0%-50% MeOH in DCM) to give the title compound (721 mg) as a tan solid.

Mass spectrum: Found [MNH₄⁺] 310

H.p.l.c. Rₜ 3.28 min

In addition to the title compound, elution of the column followed by further purification on another 40 g silica Redisep™ cartridge (eluted with 5-10% MeOH in DCM) provided a small quantity (214 mg) of 6-(2,5-dimethyl-1H-pyrrol-1-yl)-1-methyl-1,2,3,4-tetrahydroisoquinoline as a tan solid.

Mass spectrum: Found [MH⁺] 241

H.p.l.c. Rₜ 2.26 min

### Intermediate 64

### 6-(2,5-Dimethyl-1H-pyrrol-1-yl)-1-methyl-1,2,3,4-tetrahydroisoquinoline

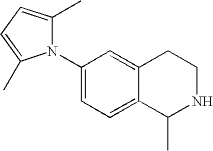

6-(2,5-Dimethyl-1H-pyrrol-1-yl)-3,4-dihydroisoquinoline:boron trifluoride complex (Intermediate 63) (732 mg) was dissolved in dry THF (20 ml) and the solution was stirred under nitrogen at room temperature. A solution of methylmagnesium bromide (1.4M solution in toluene:THF, 5 ml) was added. The solution was heated at 70° C. under nitrogen for 2 h, then left to stand at room temperature overnight. The mixture was quenched by addition of saturated aqueous ammonium chloride (5 ml) at room temperature then diluted with ethyl acetate to 200 ml then sufficient solid sodium carbonate was then added to absorb the free water. The organic phase was separated and evaporated under reduced pressure, and the crude product was purified by flash chromatography on silica (40 g Redisep™ cartridge eluted with 5%-10% MeOH in DCM). Appropriate fractions were combined and evaporated under reduced pressure to give the title compound (454 mg) as a tan solid.

Mass spectrum: Found [MH⁺] 241

H.p.l.c. Rₜ 2.26 min

### Intermediate 65

### 1-Methyl-1,2,3,4-tetrahydro-6-isoquinolinamine

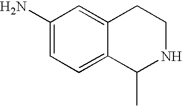

6-(2,5-Dimethyl-1H-pyrrol-1-yl)-1-methyl-1,2,3,4-tetrahydroisoquinoline (Intermediate 64) (850 mg) was dissolved in EtOH (15 ml) and water (3.75 ml) and treated with hydroxylamine hydrochloride (1.073 g) and potassium hydroxide (516 mg). The mixture was heated at reflux under nitrogen overnight. The reaction was observed to be incomplete by LCMS. EtOH (16 ml) and water (4 ml) were added followed by hydroxylamine hydrochloride (2.14 g) and potassium hydroxide (1.12 g) and the mixture was at reflux under nitrogen for a further 9 h then left at room temperature overnight. The solvents were removed under reduced pressure and the residue was taken up in 1M hydrochloric acid (100 ml) and washed with diethyl ether (3×100 ml). The aqueous phase was then basified by addition of solid sodium carbonate (caution; effervescence). Sufficient sodium carbonate was added to saturate the aqueous phase which was then extracted with DCM (200 ml). The DCM extract was separated (hydrophobic frit) and evaporated under reduced pressure, and the crude product was purified by flash chromatography on silica (40 g Redisep™ cartridge eluted with 10%-100% MeOH in DCM). Appropriate fractions were combined and evaporated under reduced pressure to give the title compound (598 mg) as a yellow gum.

Mass spectrum: Found [MH⁺] 163

H.p.l.c. Rₜ 0.34 min

### Intermediate 66

### 1,1-Dimethylethyl 6-amino-1-methyl-3,4-dihydro-2(1H)-isoquinolinecarboxylate

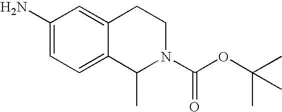

1-Methyl-1,2,3,4-tetrahydro-6-isoquinolinamine (Intermediate 65) (392 mg) was stirred with 1,4-dioxane (18 ml) and water (3 ml), di-t-butyl dicarbonate (520 mg) was added followed by triethylamine (336 ul). The mixture was stirred at room temperature overnight then the dioxane was removed under reduced pressure and the residue was diluted with water (10 ml) and extracted with DCM (2×20 ml). The organic phase was separated (hydrophobic frit) and evaporated under reduced pressure to give the title compound (492 mg) as a colourless gum

Mass spectrum: Found [MH⁺] 263

H.p.l.c. Rₜ 2.71 min

### Intermediate 67

### 1,1-Dimethylethyl 6-[(3S)-3-amino-2-oxo-1-pyrrolidinyl]-1-methyl-3,4-dihydro-2(1H)-isoquinolinecarboxylate

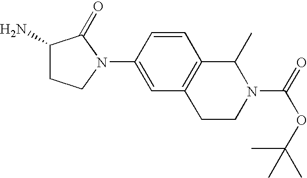

The title compound was prepared using 1,1-dimethylethyl 6-amino-1-methyl-3,4-dihydro-2(1H)-isoquinolinecarboxylate (Intermediate 66) and the procedures described for Intermediates 1, 2 and 3.

Mass spectrum: Found [MH⁺] 480

H.p.l.c. Rₜ 3.57 min

### Intermediate 68

### 1,1-Dimethylethyl 6-((3S)-3-{[(6-chloro-2-naphthalenyl)sulfonyl]amino}-2-oxo-1-pyrrolidinyl)-1-methyl-3,4-dihydro-2(1H)-isoquinolinecarboxylate

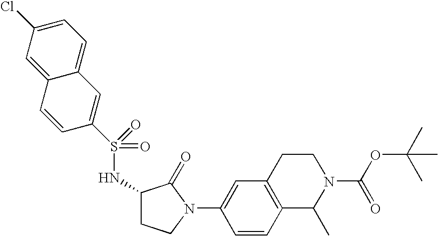

The title compound was prepared using 1,1-dimethylethyl 6-[(3S)-3-amino-2-oxo-1-pyrrolidinyl]-1-methyl-3,4-dihydro-2(1H)-isoquinolinecarboxylate (Intermediate 67) and the procedure described for Intermediate 7, employing 6-chloro-2-naphthalenesulfonyl chloride.

Mass spectrum: Found [MH⁺] 570, 572

H.p.l.c. Rₜ 3.78 min

### Intermediate 69

### 1,1-Dimethylethyl 6-[(3S)-3-({[(E)-2-(5-chloro-2-thienyl)ethenyl]sulfonyl}amino)-2-oxo-1-pyrrolidinyl]-1-methyl-3,4-dihydro-2(1H)-isoquinolinecarboxylate

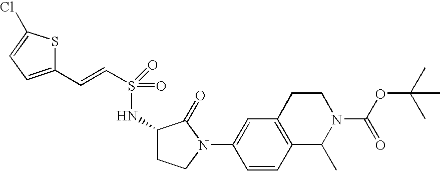

The title compound was prepared using 1,1-dimethylethyl 6-[(3S)-3-amino-2-oxo-1-pyrrolidinyl]-1-methyl-3,4-dihydro-2(1H)-isoquinolinecarboxylate (Intermediate 67) and the procedure described for Intermediate 7, employing (E)-2-(5-chloro-2-thienyl)ethenesulfonyl chloride.

Mass spectrum: Found [MH⁺] 552, 554

H.p.l.c. Rₜ 3.67 min

### Intermediate 70

### N-[1-Methyl-2-(3-nitrophenyl)ethyl]methanesulfonamide

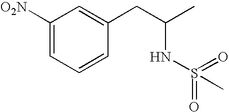

(a) A solution of methanesulfonic anhydride (0.662 g) in dry MeCN (10 ml) was added to a stirring mixture of 1-(3-nitrophenyl)-2-propanamine hydrochloride (EP1258252A1) (0.7487 g) and pyridine (0.699 ml) in dry MeCN (20 ml) at ambient temperature. After 18 h, DIPEA (1.5 ml) was added. The mixture was stirred for a further 6 h and then evaporated in vacuo. The resultant residue was quenched with saturated aqueous NaHCO₃ (20 ml), extracted with ethyl acetate (2×30 ml) and dried (MgSO₄). Evaporation in vacuo gave a pale brown oil which was loaded onto a 10 g SCX-2 cartridge preconditioned with MeOH. Elution with MeOH gave the title compound as a creamy white solid (0.547 g).

Mass spectrum: Found: MH⁺ 257

H.p.l.c. Rₜ 2.59 min

(b) Triethylamine (3 ml) was added to a stirring mixture of 1-(3-nitrophenyl)-2-propanamine hydrochloride (EP1258252A1) (2.110 g) in dry DCM (100 ml) at ambient temperature until all solid had dissolved. The solution was cooled to −20° C., then treated dropwise with methanesulfonyl chloride (830 ul). The mixture was stirred at −20° C. for a further 20 min, then allowed to warm to room temperature and extracted with 0.5M aqueous hydrochloric acid (100 ml) followed by saturated aqueous sodium hydrogen carbonate (100 ml). The organic phase was separated (hydrophobic frit), then dried (Na₂SO₄) and evaporated under reduced pressure to give the title compound (2.260 g) as an off white solid.

Mass spectrum: Found: MH⁺ 257

H.p.l.c. Rₜ 2.56 min

### Intermediate 71

### 3-Methyl-2-(methylsulfonyl)-6-nitro-1,2,3,4-tetrahydroisoquinoline

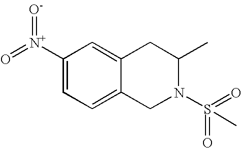

60% Concentrated sulfuric acid in glacial acetic acid (12 ml, cold) was added to N-[1-methyl-2-(3-nitrophenyl)ethyl]methanesulfonamide (Intermediate 70) (0.547 g) and stirred for 2 min to ensure a homogeneous solution. Paraformaldehyde (0.131 g) was added. The mixture was stirred at 44-48° C. for 19 h, cooled and poured into an ice-water mixture (150 g). The resultant mixture was extracted with DCM (3×30 ml), dried (MgSO₄) and evaporated in vacuo to give a light brown oil. Purification by SPE using a 12 g Redisep™ cartridge and eluted with 1:4 to 2:3 ethyl acetate:cyclohexane gave the title compound (0.488 g) containing 10% of the corresponding 8-nitro isomer (3-methyl-2-(methylsulfonyl)-8-nitro-1,2,3,4-tetrahydroisoquinoline, h.p.l.c. Rₜ 2.77 min).

Mass spectrum: Found: MH⁺ 271

H.p.l.c. Rₜ 2.81 min

### Intermediate 72

### 3-Methyl-6-nitro-1,2,3,4-tetrahydroisoquinoline

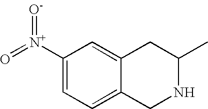

3-Methyl-2-(methylsulfonyl)-6-nitro-1,2,3,4-tetrahydroisoquinoline (Intermediate 71) (0.488 g) was heated in aqueous HBr (48%, 16 ml) at 80° C. for 4 h. The cooled aqueous solution was diluted with water (50 ml) and extracted with ethyl acetate (30 ml). The aqueous solution was then basified with 10M aqueous NaOH, extracted with DCM (3×25 ml) and dried (Na₂SO₄). Evaporation in vacuo gave the title compound (8-nitro isomer still present) as a brownish solid (0.322 g).

Mass spectrum: Found: MH⁺ 193

H.p.l.c. Rₜ 0.42 and 0.90 min

### Intermediate 73

### 1,1-Dimethylethyl 3-methyl-6-nitro-3,4-dihydro-2(1H)-isoquinolinecarboxylate

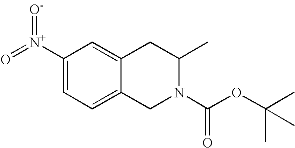

A mixture of 3-methyl-6-nitro-1,2,3,4-tetrahydroisoquinoline (Intermediate 72) (0.322 g) and di-tert-butyl dicarbonate (0.549 g) in dry DCM (5 ml) was stirred under nitrogen. Pyridine (0.272 ml) was added and stirred for 3.5 h. The mixture was evaporated in vacuo and purified on a 12 g Redisep™ silica gel cartridge eluted with DCM to afford the title compound (8-nitro isomer still present) as a creamy white solid (0.411 g).

Mass spectrum: Found: MH⁺ 293

H.p.l.c. Rₜ 3.49 min

### Intermediate 74

### 1,1-Dimethylethyl 6-amino-3-methyl-3,4-dihydro-2(1H)-isoquinolinecarboxylate

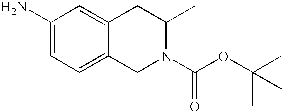

A mixture of 1,1-dimethylethyl 3-methyl-6-nitro-3,4-dihydro-2(1H)-isoquinolinecarboxylate (Intermediate 73) (0.411 g) and 10% Pd—C (41 mg) in EtOH (20 ml) was stirred at 50° C. under 50 psi of hydrogen pressure for 4 h and then 41 psi at ambient temperature overnight. The nitrogen purged mixture was filtered through celite, washed well with EtOH and evaporated in vacuo. Purification by SPE using a Redisep™ silica gel cartridge and eluted with DCM to 15% ethyl acetate-DCM afforded the title compound (8-nitro isomer still present) as a red oil (0.371 g).

Mass spectrum: Found: MH⁺ 263

H.p.l.c. Rₜ 2.68 min

### Intermediate 75

### 1,1-Dimethylethyl 6-[(3S)-3-amino-2-oxo-1-pyrrolidinyl]-3-methyl-3,4-dihydro-2(1H)-isoquinolinecarboxylate

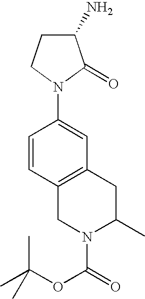

The title compound (8-nitro isomer still present) was prepared using 1,1-dimethylethyl 6-amino-3-methyl-3,4-dihydro-2(1H)-isoquinolinecarboxylate (Intermediate 74) and the procedures described for Intermediates 1, 2 and 3.

Mass spectrum: Found: MH⁺ 346

H.p.l.c. Rₜ 2.35 min

### Intermediate 76

### 1,1-Dimethylethyl 6-[(3S)-3-({[(E)-2-(5-chloro-2-thienyl)ethenyl]sulfonyl}amino)-2-oxo-1-pyrrolidinyl]-3-methyl-3,4-dihydro-2(1H)-isoquinolinecarboxylate

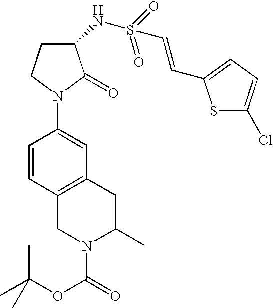

The title compound was prepared using 1,1-dimethylethyl 6-[(3S)-3-amino-2-oxo-1-pyrrolidinyl]-3-methyl-3,4-dihydro-2(1H)-isoquinolinecarboxylate (Intermediate 75) and the procedure described for Intermediate 7, employing (E)-2-(5-chloro-2-thienyl)ethenesulfonyl chloride.

Mass spectrum: Found: MH⁺ 552/554

H.p.l.c. Rₜ 3.67 min

### Intermediate 77

### 1,1-Dimethylethyl 2-amino-6,7-dihydro[1,3]thiazolo[5,4-c]pyridine-5(4H)-carboxylate

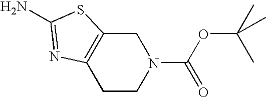

Thiourea (0.153 g) was added to a solution of 1,1-dimethylethyl 3-bromo-4-oxo-1-piperidinecarboxylate (Ref. WO2004012684) (0.557 g) in acetone (15 ml) at ambient temperature and stirred overnight. Triethylamine (418 ul) was then added and after 20 min the reaction mixture was evaporated in vacuo. The residue was loaded onto a 20 g SCX cartridge preconditioned with MeOH and then eluted with 0%-20% 2M methanolic ammonia in MeOH. Appropriate fractions were combined and evaporated in vacuo to provide a solid (0.383 g) which was stirred in water (5 ml) for 10 min, filtered, then washed with water and dried in vacuo at 45° C. to give the title compound (0.319 g) as a pale yellow solid.

Mass spectrum: Found: MH⁺ 256

H.p.l.c. Rₜ 2.09 min

### Intermediate 78

### 1,1-Dimethylethyl 2-[(3S)-2-oxo-3-({[(phenylmethyl)oxy]carbonyl}amino)-1-pyrrolidinyl]-6,7-dihydro[1,3]thiazolo[5,4-c]pyridine-5(4H)-carboxylate

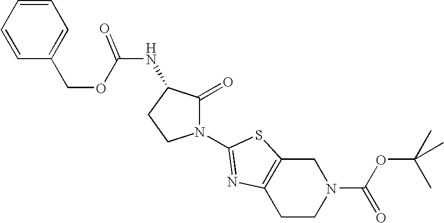

The title compound was prepared using 1,1-dimethylethyl 2-amino-6,7-dihydro[1,3]thiazolo[5,4-c]pyridine-5(4H)-carboxylate (Intermediate 77) and the procedures described for Intermediates 1 and 2.

Mass spectrum: Found: MH⁺ 473

H.p.l.c. Rₜ 3.17 min

### Intermediate 79

### 1,1-Dimethylethyl 2-[(3S)-3-amino-2-oxo-1-pyrrolidinyl]-6,7-dihydro[1,3]thiazolo[5,4-c]pyridine-5(4H)-carboxylate

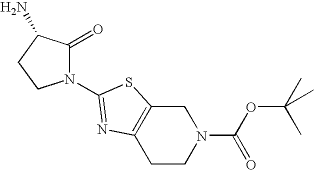

A solution of 1,1-dimethylethyl 2-[(3S)-2-oxo-3-({[(phenylmethyl)oxy]carbonyl}amino)-1-pyrrolidinyl]-6,7-dihydro[1,3]thiazolo[5,4-c]pyridine-5(4H)-carboxylate (Intermediate 78) (0.320 g) in EtOH (100 ml) was treated with batches of 10% palladium on carbon (Degussa E101 NE/W, 0.231 g in total) and ammonium formate (0.865 g in total). The mixture was stirred at 60° C. and aliquots were removed and monitored periodically by LC/MS. After a total reaction time of 8 h 25 min, further batches of ammonium formate (0.899 g in total) and 20% palladium hydroxide on carbon (Degussa E101 NE/W, 0.157 g in total) were added and the mixture was stirred at 60° C. and aliquots were removed and monitored periodically by LC/MS. After a further reaction time of 6 h 50 min, the catalyst was removed by filtration through glass fibre filters and washed with EtOH. The combined filtrate and washings were evaporated and dried in vacuo. The solid residue was loaded onto a 10 g SCX-2 cartridge preconditioned with MeOH and then eluted with 0%-100% 2M methanolic ammonia in MeOH. Appropriate fractions were combined and evaporated under reduced pressure to provide a solid which was stirred with DCM (10 ml) and then filtered. The filtrate was evaporated and the resulting pale yellow solid was dissolved in DCM (10 ml) and loaded onto a 2 g silica SPE column which was eluted successively with 100% MeOH, 1:10 MeOH:DCM, 1:1 MeOH:DCM and 100% DCM to give the title compound (0.058 g; R13279/50/13) as a pale yellow solid.

Mass spectrum: Found: MH⁺ 339

H.p.l.c. Rₜ 2.24 min

The residual solid from the above filtration was purified on a 5 g silica SPE column as described above to provide a further quantity of the title compound (0.060 g, R13279/50/15) as a white solid.

Mass spectrum: Found: MH⁺ 339

H.p.l.c. Rₜ 2.24 min

### Intermediate 80

### 1,1-Dimethylethyl 2-[(3S)-3-({[(E)-2-(5-chloro-2-thienyl)ethenyl]sulfonyl}amino)-2-oxo-1-pyrrolidinyl]-6,7-dihydro[1,3]thiazolo[5,4-c]pyridine-5(4H)-carboxylate

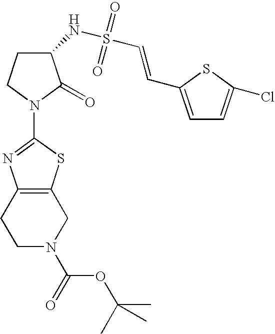

The title compound was prepared using 1,1-dimethylethyl 2-[(3S)-3-amino-2-oxo-1-pyrrolidinyl]-6,7-dihydro[1,3]thiazolo[5,4-c]pyridine-5(4H)-carboxylate (Intermediate 79) and the procedure described for Intermediate 7, employing (E)-2-(5-chloro-2-thienyl)ethenesulfonyl chloride.

Mass spectrum: Found: MH⁺ 545, 547

H.p.l.c. Rₜ 3.11 min

### Intermediate 81

### 1,1-Dimethylethyl 6-{(3S)-3-[{[(E)-2-(5-chloro-2-thienyl)ethenyl]sulfonyl}(methyl)amino]-2-oxo-1-pyrrolidinyl}-3,4-dihydro-2(1H)-isoquinolinecarboxylate

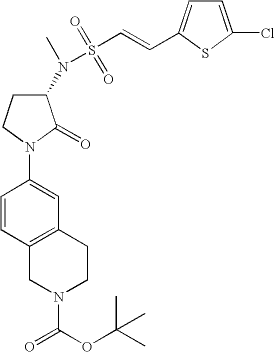

1,1-Dimethylethyl 6-[(3S)-3-({[(E)-2-(5-chloro-2-thienyl)ethenyl]sulfonyl}amino)-2-oxo-1-pyrrolidinyl]-3,4-dihydro-2(1H)-isoquinolinecarboxylate (Intermediate 8) (45 mg) was dissolved in anhydrous MeCN (1 ml). Potassium carbonate (30 mg) was added, followed by iodomethane (100 uL) and the mixture was stirred at room temperature for 64 h. The solvent was blown off using a stream of nitrogen and the residue was partitioned between DCM (2 ml) and 0.5M aqueous hydrochloric acid (3 ml). The organic phase was separated using a hydrophobic frit and blown down with a stream of nitrogen to give the title compound as a pale yellow solid (36 mg).

Mass spectrum: Found [MH⁺]⁻ 552, 554

H.p.l.c. Rₜ 3.73 min

### Intermediate 82

### 1,1-Dimethylethyl 6-((3S)-3-{[(6-chloro-2-naphthalenyl)sulfonyl]amino}-2-oxo-1-pyrrolidinyl)-3-methyl-3,4-dihydro-2(1H)-isoquinolinecarboxylate

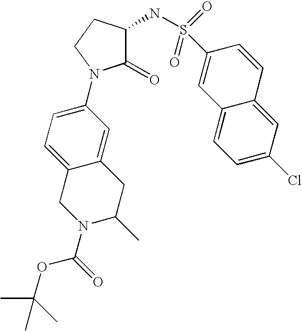

The title compound was prepared using 1,1-dimethylethyl 6-[(3S)-3-amino-2-oxo-1-pyrrolidinyl]-3-methyl-3,4-dihydro-2(1H)-isoquinolinecarboxylate (Intermediate 75) and the procedure described for Intermediate 7.

Mass spectrum: Found: MH⁺ 570, 572

H.p.l.c. Rₜ 3.78 min

### Intermediate 83

### 1,1-Dimethylethyl 6-((3S)-3-{[(6-chloro-1-{[(1,1-dimethylethyl)oxy]carbonyl}-1H-indol-2-yl)sulfonyl]amino}-2-oxo-1-pyrrolidinyl)-3,4-dihydro-2(1H)-isoquinolinecarboxylate

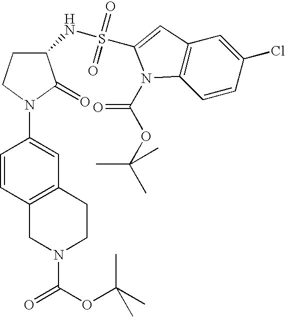

The title compound was prepared using 1,1-dimethylethyl 6-[(3S)-3-amino-2-oxo-1-pyrrolidinyl]-3,4-dihydro-2(1H)-isoquinolinecarboxylate (Intermediate 3) and the procedure described for Intermediate 7, employing 1,1-dimethylethyl 5-chloro-2-(chlorosulfonyl)-1H-indole-1-carboxylate.

Mass spectrum: Found: MH⁺ 645

H.p.l.c. Rₜ 4.04 min

### Example 1

### 6-Chloro-N-[(3S)-2-oxo-1-(1,2,3,4-tetrahydro-6-isoquinolinyl)-3-pyrrolidinyl]-2-naphthalenesulfonamide hydrochloride

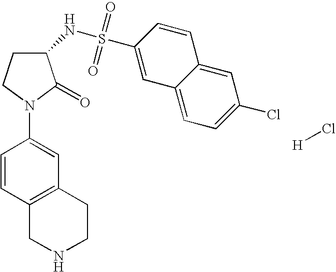

A solution of 1,1-dimethylethyl 6-((3S)-3-{[(6-chloro-2-naphthalenyl)sulfonyl]amino}-2-oxo-1-pyrrolidinyl)-3,4-dihydro-2(1H)-isoquinolinecarboxylate (Intermediate 7) (0.197 g), in MeOH (15 ml) which had previously been reacted with acetyl chloride (0.5 ml) to produce hydrogen chloride in situ, was stirred for 14 h. The title compound (0.097 g) was collected by filtration as a white solid from the reaction mixture and dried in vacuo.

Mass spectrum: Found: MH⁺ 456

H.p.l.c. Rₜ 2.56 min

¹H NMR (DMSO-d₆) δ: 1.73 (1H, m), 2.16 (1H, m), 2.96 (2H, brt), 3.38 (2H, m) 3.63 (2H, m), 4.21 (2H, s), 4.32 (1H, dd), 7.19 (1H, d), 7.40 (1H, brs), 7.50 (1H, brd), 7.70 (1H, dd) 7.96 (1H, dd) 8.12 (1H, d), 8.21 (2H, m), 8.45 (1H, m), 8.55 (1H, brs), 9.17 (2H, brs).

### Example 2

### (E)-2-(5-Chloro-2-thienyl)-N-[(3S)-2-oxo-1-(1,2,3,4-tetrahydro-6-isoquinolinyl)-3-pyrrolidinyl]ethenesulfonamide hydrochloride

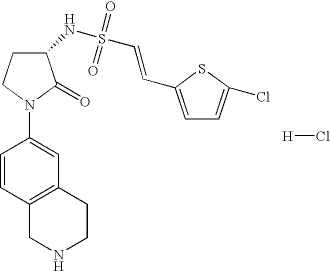

The title compound was prepared using 1,1-dimethylethyl 6-[(3S)-3-({[(E)-2-(5-chloro-2-thienyl)ethenyl]sulfonyl}amino)-2-oxo-1-pyrrolidinyl]-3,4-dihydro-2(1H)-isoquinolinecarboxylate (Intermediate 8) and the procedure described for Example 1.

Mass spectrum: Found: MH⁺ 438

H.p.l.c. Rₜ 2.39 min

¹H NMR (DMSO-d₆) δ: 1.92 (1H, m), 2.50 (1H, m), 3.00 (2H, brt), 3.40 (2H, m) 3.73 (2H, m), 4.25 (3H, m), 6.98 (1H, d), 7.20 (1H, d), 7.22 (1H, d) 7.44 (1H, m), 7.46 (1H, s) 7.50 (1H, d), 7.58 (1H, d), 8.01 (1H, d), 9.22 (2H, brs).

### Example 3

### 6-Chloro-N-[(3S)-2-oxo-1-(1,2,3,4-tetrahydro-6-isoquinolinyl)-3-pyrrolidinyl]-1-benzothiophene-2-sulfonamide hydrochloride

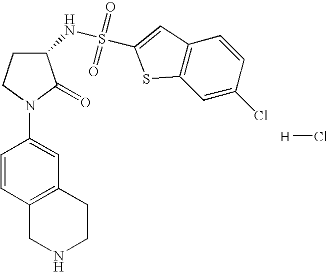

The title compound was prepared using 1,1-dimethylethyl 6-((3S)-3-{[(6-chloro-1-benzothien-2-yl)sulfonyl]amino}-2-oxo-1-pyrrolidinyl)-3,4-dihydro-2(1H)-isoquinolinecarboxylate (Intermediate 9) and the procedure described for Example 1.

Mass spectrum: Found: MH⁺ 462

H.p.l.c. Rₜ 2.70 min

¹H NMR (DMSO-d₆) δ: 1.82 (1H, m), 2.33 (1H, m), 2.96 (2H, brt), 3.40 (2H, m) 3.67 (2H, m), 4.21 (2H, s), 4.38 (1H, brt), 7.20 (1H, d), 7.43 (1H, brs), 7.54 (2H, m), 8.05 (2H, m), 8.30 (1H, s), 8.93 (3H, br).

### Example 4

### 3-Chloro-N-[(3S)-2-oxo-1-(1,2,3,4-tetrahydro-6-isoquinolinyl)-3-pyrrolidinyl]-1H-indole-6-sulfonamide formate

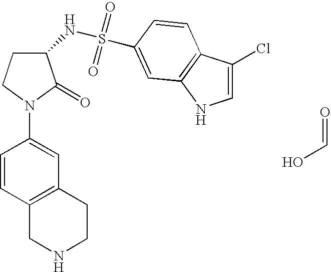

To a solution of 1,1-dimethylethyl 6-{(3S)-3-[({3-chloro-1-[tris(1-methylethyl)silyl]-1H-indol-6-yl}sulfonyl)amino]-2-oxo-1-pyrrolidinyl}-3,4-dihydro-2(1H)-isoquinolinecarboxylate (Intermediate 10) (0.045 g) in THF (5 ml) were added acetic acid (0.1 ml) and tetraethylammonium fluoride (0.014 g). After 1 h aqueous ammonium chloride (1 ml) was added, all volatiles removed and the residue partitioned between water and chloroform (5 ml each). This was passed through a hydrophobic frit and a second chloroform extract made. The combined chloroform fractions were loaded onto a 10 g silica SPE cartridge and eluted with a gradient of cyclohexane:ethyl acetate. The isolated product was then treated with the hydrogen chloride/MeOH mixture described in Example 1 and the title compound (0.022 g) isolated after mass directed preparative hplc.

Mass spectrum: Found: MH⁺ 445

H.p.l.c. Rₜ 2.39 min

¹H NMR (MeOD-d₃) δ: 1.82 (1H, m), 2.25 (1H, m), 3.06 (2H, t), 3.45 (2H, t) 3.68 (2H, m), 4.19 (1H, dd), 4.29 (2H, s), 7.17 (1H, d), 7.46 (2H, m), 7.52 (1H, s), 7.66 (2H, m), 8.04 (1H, s), 8.38 (1H, brs).

### Example 5

### N-[(3S)-2-Oxo-1-(1,2,3,4-tetrahydro-6-isoquinolinyl)-3-pyrrolidinyl]-1H-indole-6-sulfonamide formate

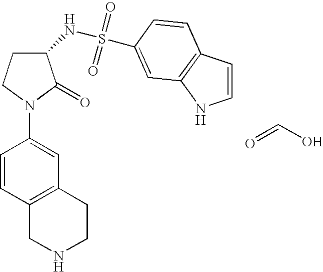

The title compound was prepared using 1,1-dimethylethyl 6-{(3S)-2-oxo-3-[({1-[tris(1-methylethyl)silyl]-1H-indol-6-yl}sulfonyl)amino]-1-pyrrolidinyl}-3,4-dihydro-2(1H)-isoquinolinecarboxylate (Intermediate 11) and the procedure described for Example 4.

Mass spectrum: Found: MH⁺ 411.

H.p.l.c. Rₜ 2.14 min

¹H NMR (MeOD-d₃) δ: 1.82 (1H, m), 2.23 (1H, m), 3.08 (2H, t), 3.46 (2H, t) 3.67 (2H, m), 4.16 (1H, dd), 4.30 (2H, s), 6.58 (1H, dd), 7.19 (1H, d), 7.47 (1H, brm), 7.49 (2H, brm), 7.57 (1H, dd), 7.72 (1H, d), 8.03 (1H, brm), 8.16 (1H, brs).

### Example 6

### 6-Chloro-N-[(3S)-2-oxo-1-(1,2,3,4-tetrahydro-6-isoquinolinyl)-3-pyrrolidinyl]-1H-indole-2-sulfonamide formate

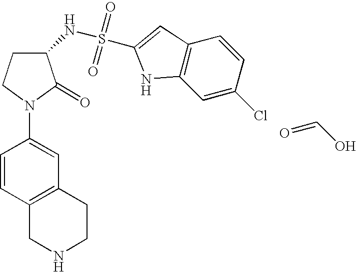

To a solution of 1,1-dimethylethyl 6-((3S)-3-{[(6-chloro-1-{[(1,1-dimethylethyl)oxy]carbonyl}-1H-indol-2-yl)sulfonyl]amino}-2-oxo-1-pyrrolidinyl)-3,4-dihydro-2(1H)-isoquinolinecarboxylate (Intermediate 12) (0.05 g) in MeOH was added concentrated hydrochloric acid (0.5 ml) and the solution stirred at ambient temperature for 14 h, then at 55° C. for 6 h. The solution was cooled, volatiles evaporated and the residue purified by mass directed preparative hplc, affording a white solid (0.018 g) after trituration with ether.

Mass spectrum: Found: MH⁺ 445

H.p.l.c. Rₜ 2.50 min

¹H NMR (MeOD-d₃) δ: 1.96 (1H, m), 2.46 (1H, m), 3.08 (2H, t), 3.45 (2H, t) 3.77 (2H, m), 4.29 (2H, s), 4.38 (1H, dd), 7.09 (1H, brs), 7.11 (1H, dd), 7.21 (1H, d) 7.50 (3H, brm), 7.62 (1H, d), 8.49 (1H, brs).

### Example 7

### (E)-2-(5-Chloro-2-thienyl)-N-[(3S)-1-(2-methyl-1,2,3,4-tetrahydro-6-isoquinolinyl)-2-oxo-3-pyrrolidinyl]ethenesulfonamide formate

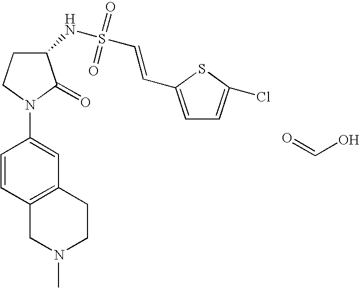

The title compound was prepared using (3S)-3-amino-1-(2-methyl-1,2,3,4-tetrahydro-6-isoquinolinyl)-2-pyrrolidinone (Intermediate 15) and (E)-2-(5-chloro-2-thienyl)ethenesulfonyl chloride following the procedure described for Intermediate 7. This was isolated as a white lyophilised solid following purification by SCX SPE, then mass directed preparative hplc and freeze drying from dioxane.

Mass spectrum: Found: MH⁺ 452

H.p.l.c. Rₜ 2.34 min

¹H NMR (MeOD-d₃) δ: 2.05 (1H, m), 2.50 (3H, s), 2.61 (1H, m), 2.81 (2H, brt), 2.96 (2H, brt) 3.67 (2H, s), 3.78 (2H, m), 4.29 (1H, dd) 6.89 (1H, d), 7.00 (1H, d), 7.10 (1H, d) 7.22 (1H, d), 7.41 (2H, brm) 7.51 (1H, d), 8.51 (1H, brs).

### Example 8

### 6-Chloro-N-[(3S)-1-(2-methyl-1,2,3,4-tetrahydro-6-isoquinolinyl)-2-oxo-3-pyrrolidinyl]-1-benzothiophene-2-sulfonamide formate

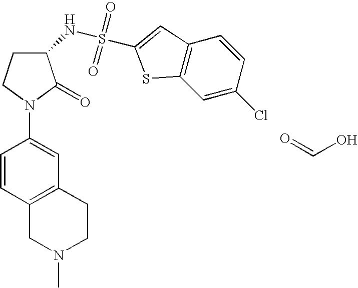

The title compound was prepared using (3S)-3-amino-1-(2-methyl-1,2,3,4-tetrahydro-6-isoquinolinyl)-2-pyrrolidinone (Intermediate 15) and 6-chloro-1-benzothiophene-2-sulfonyl chloride following the procedure described for Intermediate 7. This was isolated as a white lyophilised solid following purification by SCX SPE, then mass directed preparative hplc and freeze drying from dioxane.

Mass spectrum: Found: MH⁺ 476

H.p.l.c. Rₜ 2.55 min

¹H NMR (MeOD-d₃) δ: 1.96 (1H, m), 2.47 (1H, m), 2.55 (3H, s) 2.89 (2H, brm), 2.96 (2H, brm) 3.74 (4H, m), 4.37 (1H, dd), 7.08 (1H, d), 7.38 (2H, brm), 7.47 (1H, dd), 7.91 (1H, dd), 8.01 (2H, brm), 8.51 (1H, br).

### Example 9

### 5-Chloro-N-[(3S)-2-oxo-1-(1,2,3,4-tetrahydro-6-isoquinolinyl)-3-pyrrolidinyl]-1-benzothiophene-2-sulfonamide trifluoroacetate

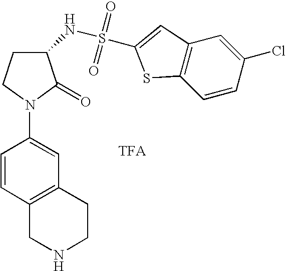

The title compound was prepared using 1,1-dimethylethyl 6-((3S)-3-{[(5-chloro-1-benzothien-2-yl)sulfonyl]amino}-2-oxo-1-pyrrolidinyl)-3,4-dihydro-2(1H)-isoquinolinecarboxylate (Intermediate 16) and the procedure described for Example 16, followed by purification by preparative HPLC.

Mass spectrum: Found: MH⁺ 462

H.p.l.c. Rₜ 2.46 min

¹H NMR (MeOD-d₃) δ: 1.98 (1H, m), 2.49 (1H, m), 3.08 (2H, t), 3.47 (2H, t) 3.76 (2H, m), 4.31 (2H, s), 4.40 (1H, dd), 7.21 (1H, d), 7.50 (3H, m), 7.95 (1H, d), 7.98 (2H, brm).

### Example 10

### 5′-Chloro-N-[(3S)-2-oxo-1-(1,2,3,4-tetrahydro-6-isoquinolinyl)-3-pyrrolidinyl]-2,2′-bithiophene-5-sulfonamide hydrochloride

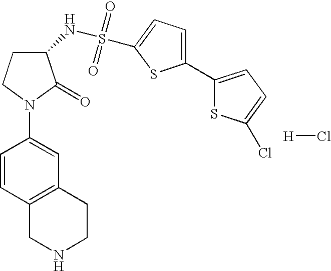

The title compound was prepared using 1,1-dimethylethyl 6-((3S)-3-{[(5′-chloro-2,2′-bithien-5-yl)sulfonyl]amino}-2-oxo-1-pyrrolidinyl)-3,4-dihydro-2(1H)-isoquinolinecarboxylate (Intermediate 17) and the procedure described for Example 16.

Mass spectrum: Found: MH⁺ 494

H.p.l.c. Rₜ 2.67 min

¹H NMR (DMSO-d₆) δ: 1.82 (1H, m), 2.31 (1H, m), 2.99 (2H, brt), 3.36 (2H, m) 3.69 (2H, m), 4.22 (2H, s), 4.35 (1H, dd), 7.21 (1H, d), 7.22 (1H, d) 7.38 (1H, d), 7.40 (1H, d), 7.46 (1H, brd), 7.53 (1H, dd) 7.64 (1H, d) 8.66 (1H, d), 9.23 (2H, brs).

### Example 11

### 6-Chloro-N-[(3S)-2-oxo-1-(1,2,3,4-tetrahydro-7-isoquinolinyl)-3-pyrrolidinyl]-2-naphthalenesulfonamide hydrochloride

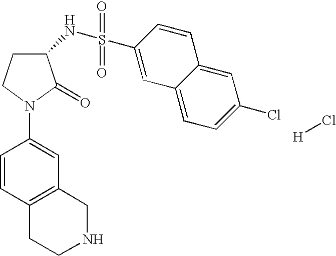

The title compound was prepared using 1,1-dimethylethyl 7-((3S)-3-{[(6-chloro-2-naphthalenyl)sulfonyl]amino}-2-oxo-1-pyrrolidinyl)-3,4-dihydro-2(1H)-isoquinolinecarboxylate (Intermediate 19) and the procedure described for Example 16.

Mass spectrum: Found: MH⁺ 456

H.p.l.c. Rₜ 2.56 min

¹H NMR (DMSO-d₆) δ: 1.73 (1H, m), 2.16 (1H, m), 2.96 (2H, brt), 3.38 (2H, m) 3.63 (2H, m), 4.21 (2H, s), 4.32 (1H, dd), 7.19 (1H, d), 7.40 (1H, brs), 7.50 (1H, brd), 7.70 (1H, dd) 7.96 (1H, dd) 8.12 (1H, d), 8.21 (2H, m), 8.45 (1H, m), 8.55 (1H, brs), 9.17 (2H, brs).

### Example 12

### 2-(5-Chloro-2-thienyl)-N-[(3S)-2-oxo-1-(1,2,3,4-tetrahydro-7-isoquinolinyl)-3-pyrrolidinyl]ethanesulfonamide hydrochloride

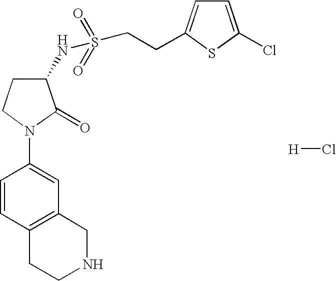

The title compound was prepared using 1,1-dimethylethyl 7-[(3S)-3-({[2-(5-chloro-2-thienyl)ethyl]sulfonyl}amino)-2-oxo-1-pyrrolidinyl]-3,4-dihydro-2(1H)-isoquinolinecarboxylate (Intermediate 20) and the procedure described for Example 1.

Mass spectrum: Found: MH⁺ 440

H.p.l.c. Rₜ 2.41 min

¹H NMR (MeOD-d₃) δ: 2.05 (1H, m), 2.62 (1H, m), 3.10 (2H, t), 3.26 (1H, m) 3.42 (1H, m) 3.50 (3H, m), 3.61 (1H, m), 3.82 (2H, m) 4.36 (2H, s), 4.42 (1H, dd), 6.79 (2H, m), 7.29 (1H, d), 7.57 (1H, m), 7.60 (1H, brs).

### Example 13

### (E)-2-(5-Chloro-2-thienyl)-N-[(3S)-2-oxo-1-(1,2,3,4-tetrahydro-7-isoquinolinyl)-3-pyrrolidinyl]ethenesulfonamide hydrochloride

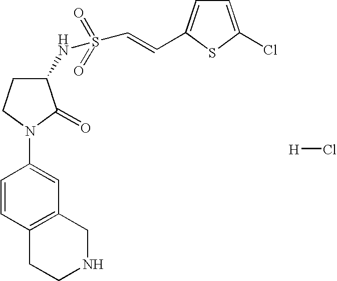

The title compound was prepared using 1,1-dimethylethyl 7-[(3S)-3-({[(E)-2-(5-chloro-2-thienyl)ethenyl]sulfonyl}amino)-2-oxo-1-pyrrolidinyl]-3,4-dihydro-2(1H)-isoquinolinecarboxylate (Intermediate 21) and the procedure described for Example 1.

Mass spectrum: Found: MH⁺ 438

H.p.l.c. Rₜ 2.40 min

¹H NMR (DMSO-d₆) δ: 1.93 (1H, m), 2.50 (1H, m), 3.00 (2H, brt), 3.40 (2H, m) 3.73 (2H, m), 4.25 (3H, m), 6.98 (1H, d), 7.20 (1H, d), 7.24 (1H, d) 7.44 (1H, d), 7.46 (1H, brs) 7.50 (1H, d), 7.59 (1H, d), 8.02 (1H, d), 9.28 (2H, brs).

### Example 14

### 3-Chloro-N-[(3S)-2-oxo-1-(1,2,3,4-tetrahydro-7-isoquinolinyl)-3-pyrrolidinyl]-1H-indole-6-sulfonamide formate

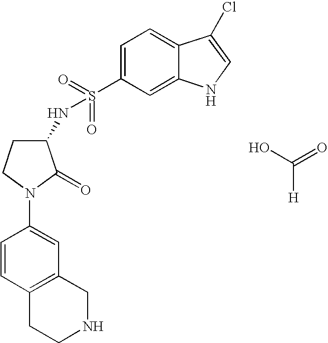

A solution of 1,1-dimethylethyl 7-{(3S)-3-[({3-chloro-1-[tris(1-methylethyl)silyl]-1H-indol-6-yl}sulfonyl)amino]-2-oxo-1-pyrrolidinyl}-3,4-dihydro-2(1H)-isoquinolinecarboxylate (Intermediate 22) (0.109 g) was stirred for 14 h in a mixture of trifluoroacetic acid (10 ml) and water (1 ml), after which the volatile components were evaporated. The title compound (0.010 g) was isolated after mass spectrum directed preparative h.p.l.c.

Mass spectrum: Found: MH⁺ 445

H.p.l.c. Rₜ 2.44 min

¹H NMR (MeOD-d₃) δ: 1.82 (1H, m), 2.24 (1H, m), 3.05 (2H, t), 3.46 (2H, t) 3.67 (2H, m), 4.19 (1H, dd), 4.30 (2H, s), 7.21 (1H, d), 7.45 (2H, m), 7.52 (1H, s), 7.66 (2H, m), 8.04 (1H, s), 8.16 (1H, brs).

### Example 15

### N-[(3S)-2-Oxo-1-(1,2,3,4-tetrahydro-7-isoquinolinyl)-3-pyrrolidinyl]-1H-indole-6-sulfonamide formate

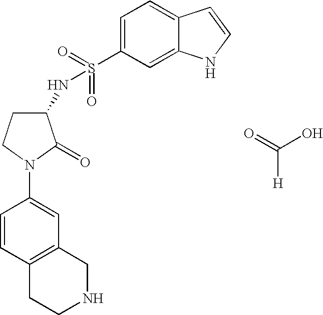

The title compound was prepared using 1,1-dimethylethyl 7-{(3S)-2-oxo-3-[({1-[tris(1-methylethyl)silyl]-1H-indol-6-yl}sulfonyl)amino]-1-pyrrolidinyl}-3,4-dihydro-2(1H)-isoquinolinecarboxylate (Intermediate 23) and the procedure described for Example 14.

Mass spectrum: Found: MH⁺ 411

H.p.l.c. Rₜ 2.17 min

¹H NMR (MeOD-d₃) δ: 1.83 (1H, m), 2.25 (1H, m), 3.06 (2H, t), 3.47 (2H, t) 3.67 (2H, m), 4.16 (1H, dd), 4.31 (2H, s), 6.58 (1H, brm), 7.23 (1H, d), 7.45 (1H, dd), 7.49 (3H, brm), 7.58 (1H, dd), 7.72 (1H, d), 8.03 (1H, brs).

### Example 16

### 6-Chloro-N-[(3R)-2-oxo-1-(1,2,3,4-tetrahydro-6-isoquinolinyl)-3-pyrrolidinyl]-2-naphthalenesulfonamide hydrochloride

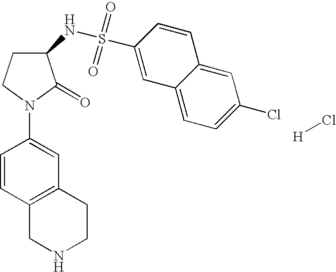

A solution of 1,1-dimethylethyl 6-((3R)-3-{[(6-chloro-2-naphthalenyl)sulfonyl]amino}-2-oxo-1-pyrrolidinyl)-3,4-dihydro-2(1H)-isoquinolinecarboxylate (Intermediate 25) (0.276 g) in 4M HCl in dioxane (15 ml) was stirred for 14 h, after which the volatiles were evaporated. The residue was stirred with diethyl ether and the title compound (0.190 g) was collected by filtration and dried in vacuo.

Mass spectrum: Found: MH⁺ 456

H.p.l.c. Rₜ 2.61 min

¹H NMR (DMSO-d₆) δ: 1.73 (1H, m), 2.16 (1H, m), 2.95 (2H, brt), 3.35 (2H, m) 3.63 (2H, m), 4.20 (2H, s), 4.32 (1H, dd), 7.19 (1H, d), 7.40 (1H, brs), 7.50 (1H, brd), 7.70 (1H, dd) 7.96 (1H, dd) 8.12 (1H, d), 8.21 (2H, m), 8.45 (1H, m), 8.55 (1H, brs), 8.95 (2H, brs).

### Example 17

### 6-Chloro-N-[(3S)-1-(5-fluoro-1,2,3,4-tetrahydro-6-isoquinolinyl)-2-oxo-3-pyrrolidinyl]-2-naphthalenesulfonamide hydrochloride

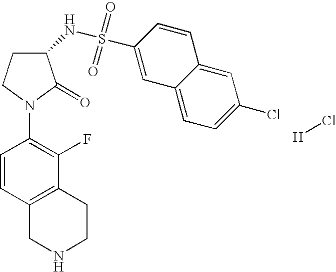

The title compound was prepared using 1,1-dimethylethyl 6-((3S)-3-{[(6-chloro-2-naphthalenyl)sulfonyl]amino}-2-oxo-1-pyrrolidinyl)-5-fluoro-3,4-dihydro-2(1H)-isoquinolinecarboxylate (Intermediate 32) and the procedure described for Example 16.

Mass spectrum: Found: MH⁺ 474

H.p.l.c. Rₜ 2.64 min

¹H NMR (MeOD-d₃) δ: 1.99 (1H, m), 2.40 (1H, m), 3.03 (2H, t), 3.48 (2H, t) 3.63 (1H, m), 3.72 (1H, m) 4.34 (2H, s), 4.35 (1H, dd), 7.08 (1H, d), 7.28 (1H, t), 7.60 (1H, dd), 8.02 (4H, m), 8.54 (1H, s).

### Example 18

### (E)-2-(5-Chloro-2-thienyl)-N-[(3S)-1-(5-fluoro-1,2,3,4-tetrahydro-6-isoquinolinyl)-2-oxo-3-pyrrolidinyl]ethenesulfonamide hydrochloride

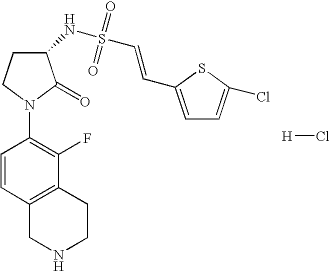

The title compound was prepared using 1,1-dimethylethyl 6-[(3S)-3-({[(E)-2-(5-chloro-2-thienyl)ethenyl]sulfonyl}amino)-2-oxo-1-pyrrolidinyl]-5-fluoro-3,4-dihydro-2(1H)-isoquinolinecarboxylate (Intermediate 33) and the procedure described for Example 16.

Mass spectrum: Found: MH⁺ 456

H.p.l.c. Rₜ 2.44 min

¹H NMR (DMSO-d₆) δ: 2.02 (1H, m), 2.50 (1H, m), 2.95 (2H, brt), 3.40 (2H, brm) 3.61 (1H, m), 3.72 (1H, m), 4.24 (1H, m), 4.28 (2H, brs), 6.98 (1H, d), 7.13 (1H, d), 7.19 (1H, d), 7.34 (1H, t), 7.42 (1H, d) 7.50 (1H, d), 8.05 (1H, d), 9.45 (2H, brs).

### Example 19

### 3-Chloro-N-[(3S)-1-(5-fluoro-1,2,3,4-tetrahydro-6-isoquinolinyl)-2-oxo-3-pyrrolidinyl]-1H-indole-6-sulfonamide hydrochloride

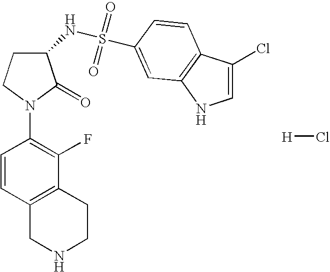

The title compound was prepared using 1,1-dimethylethyl 6-{(3S)-3-[({3-chloro-1-[tris(1-methylethyl)silyl]-1H-indol-6-yl}sulfonyl)amino]-2-oxo-1-pyrrolidinyl}-5-fluoro-3,4-dihydro-2(1H)-isoquinolinecarboxylate (Intermediate 34) and the two-step procedure described for Example 4, utilising 4M HCl in dioxane in the final stage instead of HCl in MeOH but without use of mass directed preparative HPLC.

Mass spectrum: Found: MH⁺ 463

H.p.l.c. Rₜ 2.41 min

¹H NMR (DMSO-d₆) δ: 1.73 (1H, m), 2.02 (1H, m), 2.93 (2H, t), 3.35 (2H, obs) 3.48 (1H, m), 3.85 (1H, m), 4.18 (1H, dd), 4.27 (2H, brs), 7.11 (1H, d), 7.28 (1H, t), 7.60 (1H, dd), 7.67 (1H, d), 7.82 (1H, d), 7.99 (1h, brs), 8.21 (1H, d), 9.45 (2H, brs), 11.95 (1H, s).

### Example 20

### 6-Chloro-N-[1-(7-fluoro-1,2,3,4-tetrahydro-6-isoquinolinyl)-2-oxo-3-pyrrolidinyl]-2-naphthalenesulfonamide hydrochloride

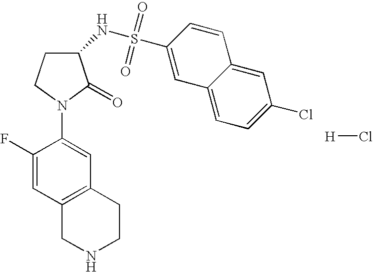

The title compound was prepared using 1,1-dimethylethyl 6-(3-{[(6-chloro-2-naphthalenyl)sulfonyl]amino}-2-oxo-1-pyrrolidinyl)-7-fluoro-3,4-dihydro-2(1H)-isoquinolinecarboxylate (Intermediate 42) and the procedure described for Example 1.

Mass spectrum: Found MH⁺ 474

H.p.l.c. Rₜ 2.58 min

¹H NMR (DMSO-d₆) δ: 1.80 (1H, m), 2.15 (1H, m), 2.93 (2H, m), 3.31 (2H, m, obs), 3.51 (1H, t), 3.61 (1H, m), 4.21 (2H, bs), 4.27 (1H, m), 7.22 (2H, m), 7.68 (1H, m), 7.96 (1H, m), 8.12 (1H, m), 8.21 (3H, m), 8.50 (1H, d), 8.56 (1H, d), 9.64 (2H, bs)

### Example 21

### 6-Chloro-N-[(3S)-1-(7-fluoro-1,2,3,4-tetrahydro-6-isoquinolinyl)-2-oxo-3-pyrrolidinyl]-1-benzothiophene-2-sulfonamide hydrochloride

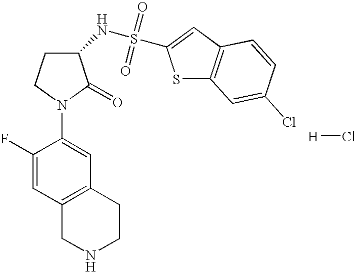

The title compound was prepared using 1,1-dimethylethyl 6-((3S)-3-{[(6-chloro-1-benzothien-2-yl)sulfonyl]amino}-2-oxo-1-pyrrolidinyl)-7-fluoro-3,4-dihydro-2(1H)-isoquinolinecarboxylate (Intermediate 43) and the procedure described for Example 1.

Mass spectrum: Found MH⁺ 480

H.p.l.c. Rₜ 2.58 min

¹H NMR (DMSO-d₆) δ: 1.89 (1H, m), 2.29 (1H, m), 2.93 (2H, m), 3.34 (2H, obs), 3.55 (1H, t), 3.66 (1H, m), 4.23 (2H, bs), 4.34 (1H, m), 7.24 (2H, m), 7.54 (1H, m), 8.05 (2H, m), 8.30 (1H, d), 8.86 (1H, bs), 9.34 (2H, bs)

### Example 22

### (E)-2-(5-Chloro-2-thienyl)-N-[(3S)-1-(7-fluoro-1,2,3,4-tetrahydro-6-isoquinolinyl)-2-oxo-3-pyrrolidinyl]ethenesulfonamide hydrochloride

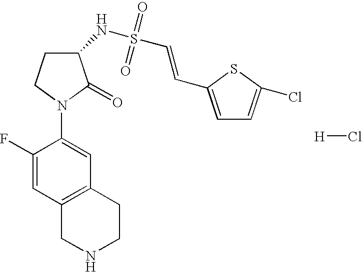

The title compound was prepared using 1,1-dimethylethyl 6-[(3S)-3-({[(E)-2-(5-chloro-2-thienyl)ethenyl]sulfonyl}amino)-2-oxo-1-pyrrolidinyl]-7-fluoro-3,4-dihydro-2(1H)-isoquinoline carboxylate (Intermediate 44) and the procedure described for Example 1.

Mass spectrum: Found MH⁺ 456

H.p.l.c. Rₜ 2.39 min

¹H NMR (DMSO-d₆) δ: 2.01 (1H, m), 2.52 (1H, obs), 2.95 (2H, m), 3.34 (2H, obs), 3.60 (1H, m), 3.71 (1H, m), 4.23 (3H, m), 6.98 (1H, d, J=15 Hz), 7.19 (1H, d), 7.27 (2H, m), 7.42 (1H, d), 7.50 (1H, d), 9.35 (2H, bs)

### Example 23

### 3-Chloro-N-[(3S)-1-(7-fluoro-1,2,3,4-tetrahydro-6-isoquinolinyl)-2-oxo-3-pyrrolidinyl]-1H-indole-6-sulfonamide hydrochloride

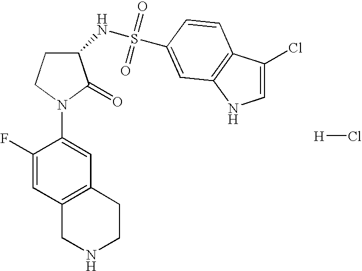

A solution of 1-dimethylethyl 6-{(3S)-3-[({3-chloro-1-[tris(1-methylethyl)silyl]-1H-indol-6-yl}sulfonyl)amino]-2-oxo-1-pyrrolidinyl}-7-fluoro-3,4-dihydro-2(1H)-isoquinolinecarboxylate (Intermediate 45) (162 mg) in THF (50 ml) and glacial acetic acid (1 ml) was treated with tetraethylammonium fluoride hydrate (50.4 mg) and the solution was stirred at room temperature for 3 h. Saturated aqueous ammonium chloride solution was added and the mixture was stirred for 30 min and then evaporated to near dryness. The residue was suspended in MeOH and purified on a 20 g SCX column. The product fractions were combined and purified on a 20 g silica column eluted with a DCM:2N ammonia in MeOH gradient to give 3-chloro-N-[(3S)-1-(7-fluoro-1,2,3,4-tetrahydro-6-isoquinolinyl)-2-oxo-3-pyrrolidinyl]-1H-indole-6-sulfonamide as a colourless solid. The amine was dissolved in anhydrous MeOH (10 ml) and treated with a solution of acetyl chloride (200 ul) in MeOH (10 ml) and stirred for 2 h. The solution was then evaporated to dryness. The residue was dissolved in MeOH (3 ml) and diluted with diethyl ether and the solid was collected by filtration to give the product as a colourless solid (76.2 mg).

Mass spectrum: Found MH⁺ 463

H.p.l.c. Rₜ 2.39 min

¹H NMR (DMSO-d₆) δ: 1.73 (1H, m), 2.04 (1H, m), 2.93 (2H, m), 3.35 (2H, m, obs), 3.52 (2H, m), 4.15 (1H, m), 4.23 (2H, s), 7.22 (2H, m), 7.63 (2H, m), 7.82 (1H, d), 7.98 (1H, s), 8.19 (1H, d), 9.28 (2H, bs), 11.95 (1H, s)

### Example 24

### 6-Chloro-N-[(3S)-1-(7-chloro-1,2,3,4-tetrahydro-6-isoquinolinyl)-2-oxo-3-pyrrolidinyl]-2-naphthalenesulfonamide hydrochloride

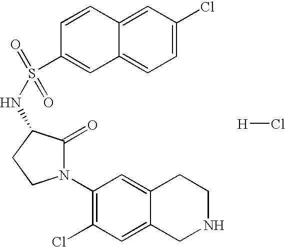

The title compound was prepared using 1,1-dimethylethyl 7-chloro-6-((3S)-3-{[(6-chloro-2-naphthalenyl)sulfonyl]amino}-2-oxo-1-pyrrolidinyl)-3,4-dihydro-2(1H)-isoquinolinecarboxylate (Intermediate 50) and the procedure described for Example 1.

Mass spectrum: Found MH⁺ 490, 492

H.p.l.c. Rₜ 2.68 min

¹H NMR (DMSO-d₆) δ: 1.82 (1H, m), 2.16 (1H, m), 2.94 (2H, brt), 3.33 (2H, m) 3.48 (2H, m), 4.24 (2H, s), 4.25 (1H, m), 7.22 (1H, s), 7.47 (1H, s), 7.70 (1H, m), 7.96 (1H, m), 8.12 (1H, d), 8.22 (2H, m), 8.50 (1H, d), 8.57 (1H, brs), 9.37 (2H, brs).

### Example 25

### (E)-N-[(3S)-1-(7-Chloro-1,2,3,4-tetrahydro-6-isoquinolinyl)-2-oxo-3-pyrrolidinyl]-2-(5-chloro-2-thienyl)ethenesulfonamide hydrochloride

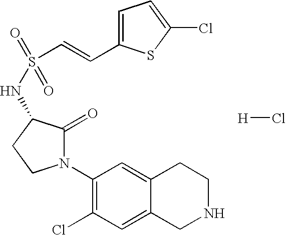

The title compound was prepared using 1,1-dimethylethyl 7-chloro-6-[(3S)-3-({[(E)-2-(5-chloro-2-thienyl)ethenyl]sulfonyl}amino)-2-oxo-1-pyrrolidinyl]-3,4-dihydro-2(1H)-isoquinolinecarboxylate (Intermediate 51) and the procedure described for Example 1.

Mass spectrum: Found MH⁺ 472, 474

H.p.l.c. Rₜ 2.48 min

¹H NMR (DMSO-d₆) δ: 2.05 (1H, m), 2.52 (1H, m), 2.97 (2H, brt), 3.35 (2H, obs.) 3.58 (2H, m), 4.22 (1H, dd), 4.26 (2H, brs), 6.99 (1H, d), 7.19 (1H, d), 7.28 (1H, s) 7.43 (1H, d), 7.50 (1H, s) 7.50 (1H, d), 8.06 (1H, d), 9.46 (2H, brs).

### Example 26

### 3-Chloro-N-[(3S)-1-(7-chloro-1,2,3,4-tetrahydro-6-isoquinolinyl)-2-oxo-3-pyrrolidinyl]-1H-indole-6-sulfonamide hydrochloride

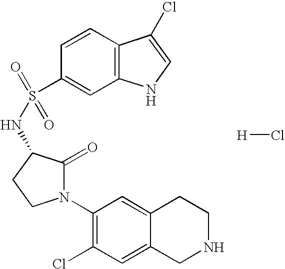

The title compound was prepared from 1,1-dimethylethyl 7-chloro-6-{(3S)-3-[({3-chloro-1-[tris(1-methylethyl)silyl]-1H-indol-6-yl}sulfonyl)amino]-2-oxo-1-pyrrolidinyl}-3,4-dihydro-2(1H)-isoquinolinecarboxylate (Intermediate 52) using the procedures described for Example 4, isolated as a hydrochloride without chromatography.

Mass spectrum: Found MH⁺ 479, 481

H.p.l.c. Rₜ 2.49 min

¹H NMR (DMSO-d₆) δ: 1.76 (1H, m), 2.04 (1H, m), 2.94 (2H, brt), 3.34 (2H, m) 3.46 (2H, m), 4.13 (1H, dd), 4.24 (2H, s), 7.22 (1H, s), 7.47 (1H, s), 7.61 (1H, dd), 7.66 (1H, d), 7.82 (1H, d), 7.99 (1H, s), 8.21 (1H, d), 9.34 (2H, brs), 11.96 (1H, brs).

### Example 27

### 6-Chloro-N-[(3S)-1-(7-methyl-1,2,3,4-tetrahydro-6-isoquinolinyl)-2-oxo-3-pyrrolidinyl]-2-naphthalenesulfonamide hydrochloride

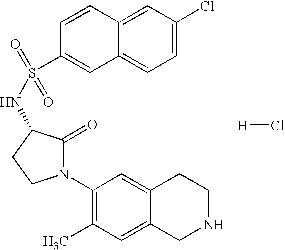

The title compound was prepared using 1,1-dimethylethyl 6-((3S)-3-{[(6-chloro-2-naphthalenyl)sulfonyl]amino}-2-oxo-1-pyrrolidinyl)-7-methyl-3,4-dihydro-2(1H)-isoquinolinecarboxylate (Intermediate 55) and the procedure described for Example 1.

Mass spectrum: Found MH⁺ 470, 472

H.p.l.c. Rₜ 2.54 min

¹H NMR (DMSO-d₆) δ: 1.79 (1H, m), 2.05 (3H, s), 2.14 (1H, m), 2.90 (2H, brt), 3.31 (2H, brt) 3.38 (1H, m), 3.52 (1H, m), 4.19 (2H, s), 4.24 (1H, m), 6.99 (1H, s), 7.08 (1H, s), 7.70 (1H, dd), 7.96 (1H, dd), 8.12 (1H, d), 8.22 (2H, m), 8.46 (1H, d), 8.57 (1H, brs), 9.23 (2H, brs).

### Example 28

### (E)-2-(5-Chloro-2-thienyl)-N-[(3S)-1-(7-methyl-1,2,3,4-tetrahydro-6-isoquinolinyl)-2-oxo-3-pyrrolidinyl]ethenesulfonamide hydrochloride

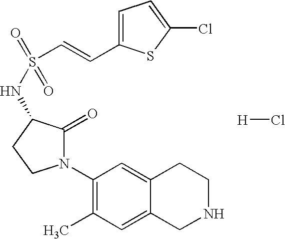

The title compound was prepared using 1,1-dimethylethyl 6-[(3S)-3-({[(E)-2-(5-chloro-2-thienyl)ethenyl]sulfonyl}amino)-2-oxo-1-pyrrolidinyl]-7-methyl-3,4-dihydro-2(1H)-isoquinolinecarboxylate (Intermediate 56) and the procedure described for Example 1.

Mass spectrum: Found MH⁺ 452, 454

H.p.l.c. Rₜ 2.38 min

¹H NMR (DMSO-d₆) δ: 2.03 (1H, m), 2.10 (3H, s), 2.50 (1H, m), 2.93 (2H, brt), 3.35 (2H, obs.) 3.48 (1H, m), 3.64 (1H, m), 4.21 (1H, m), 4.22 (2H, brs), 6.99 (1H, d), 7.06 (1H, s), 7.12 (1H, s) 7.19 (1H, d), 7.43 (1H, d) 7.51 (1H, d), 8.03 (1H, d), 9.25 (2H, brs).

### Example 29

### 3-Chloro-N-[(3S)-1-(7-methyl-1,2,3,4-tetrahydro-6-isoquinolinyl)-2-oxo-3-pyrrolidinyl]-1H-indole-6-sulfonamide hydrochloride

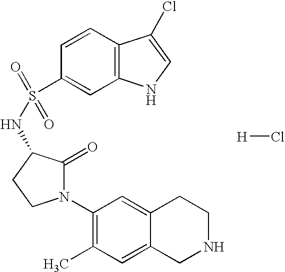

The title compound was prepared from 1,1-dimethylethyl 6-{(3S)-3-[({3-chloro-1-[tris(1-methylethyl)silyl]-1H-indol-6-yl}sulfonyl)amino]-2-oxo-1-pyrrolidinyl}-7-methyl-3,4-dihydro-2(1H)-isoquinolinecarboxylate (Intermediate 57) using the procedures described for Example 4, isolated as a hydrochloride without chromatography.

Mass spectrum: Found MH⁺ 459, 461

H.p.l.c. Rₜ 2.53 min

¹H NMR (DMSO-d₆) δ: 1.73 (1H, m), 2.02 (1H, m), 2.06 (3H, s), 2.91 (2H, brt), 3.32 (2H, m) 3.38 (1H, m), 3.50 (1H, m), 4.14 (1H, dd), 4.19 (2H, s), 7.00 (1H, s), 7.09 (1H, s), 7.60 (1H, dd), 7.66 (1H, d), 7.83 (1H, d), 7.99 (1H, s), 8.17 (1H, d), 9.27 (2H, brs), 11.97 (1H, brs).

### Example 30

### 6-Chloro-N-{(3S)-1-[2-(1-methylethyl)-1,2,3,4-tetrahydro-6-isoquinolinyl]-2-oxo-3-pyrrolidinyl}-2-naphthalenesulfonamide

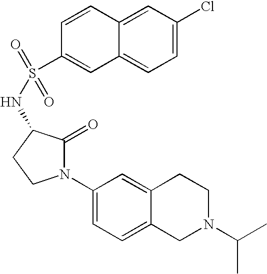

A solution of the free base of 6-chloro-N-[(3S)-2-oxo-1-(1,2,3,4-tetrahydro-6-isoquinolinyl)-3-pyrrolidinyl]-2-naphthalenesulfonamide hydrochloride (Example 1) (200 mg, prepared by passing the hydrochloride through a 20 g silica SPE cartridge, eluted with 9:1 chloroform:2M ammonia in MeOH) in DCM (10 ml) was stirred for 20 min at ambient temperature with acetone (0.052 ml) and glacial acetic acid. Tetramethylammonium triacetoxyborohydride (0.232 g) was added and the solution stirred for a further 14 h at ambient temperature before a second portion (0.116 g) of this reagent was added and stirring continued for another 72 h. Completion was evident 2 h after a third portion (0.108 g) of the reagent was added and the reaction was quenched with 1 ml of saturated aqueous ammonium chloride. The mixture was treated with 50 ml of saturated aqueous sodium carbonate, passed through a hydrophobic frit and extracted twice with DCM. The title compound (0.162 g) was isolated as a white solid after purification on a 20 g silica SPE cartridge, eluted with ethyl acetate then 100:10:1 DCM:MeOH:0.88 ammonia.

Mass spectrum: Found MH⁺ 498, 500

H.p.l.c. Rₜ 2.86 min

¹H NMR (CDCl₃) δ: 1.13 (6H, d), 2.18 (1H, m), 2.74 (3H, m), 2.86 (3H, m), 3.68 (2H, brs) 3.75 (2H, m), 3.87 (1H, dd), 7.02 (1H, m), 7.27 (2H, obsc) 7.58 (1H, m), 7.94 (4H, m), 8.48 (1H, brs).

### Example 31

### 6-Chloro-N-[(3S)-1-(1-methyl-1,2,3,4-tetrahydro-6-isoquinolinyl)-2-oxo-3-pyrrolidinyl]-2-naphthalenesulfonamide hydrochloride

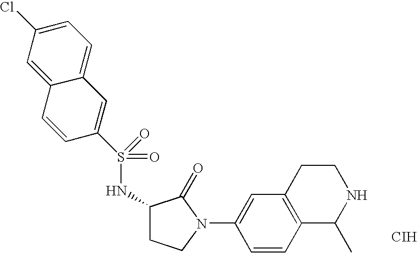

1,1-Dimethylethyl 6-((3S)-3-{[(6-chloro-2-naphthalenyl)sulfonyl]amino}-2-oxo-1-pyrrolidinyl)-1-methyl-3,4-dihydro-2(1H)-isoquinolinecarboxylate (Intermediate 68) (201 mg) was stirred in a solution of hydrogen chloride in 1,4-dioxane (15 ml of 4M solution) at room temperature for 1.5 h. The resulting suspension was blown down overnight using a stream of nitrogen and the residual solid was stirred with diethyl ether (25 ml) then filtered with suction. The solid was washed with diethyl ether (2×20 ml) then dried with suction to give the title compound as a white solid (144 mg).

Mass spectrum: Found [MH⁺] 470,472

H.p.l.c. Rₜ 2.68 min

¹H NMR (DMSO-d₆) δ: 1.55 (3H, d); 1.67-1.80 (1H, m); 2.11-2.20 (1H, m); 2.88-3.08 (2H, m); 3.20-3.45 (2H, br m); 3.60-3.67 (2H, m); 4.27-4.36 (1H, m); 4.49 (1H, br s); 7.27 (1H, d); 7.37 (1H, br s); 7.51 (1H, br d); 7.69 (1H, dd); 7.96 (1H, dd); 8.12 (1H, d); 8.19-8.23 (2H, m); 8.46 (1H, d); 8.55 (1H, s); 9.26 (1H, br s); 9.72 (1H, br s).

### Example 32

### (E)-2-(5-Chloro-2-thienyl)-N-[(3S)-1-(1-methyl-1,2,3,4-tetrahydro-6-isoquinolinyl)-2-oxo-3-pyrrolidinyl]ethenesulfonamide hydrochloride

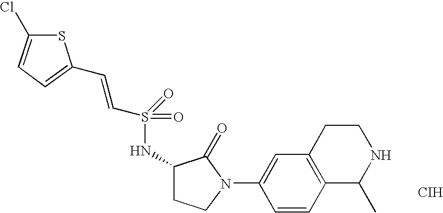

The title compound was prepared using 1,1-dimethylethyl 6-[(3S)-3-({[(E)-2-(5-chloro-2-thienyl)ethenyl]sulfonyl}amino)-2-oxo-1-pyrrolidinyl]-1-methyl-3,4-dihydro-2(1H)-isoquinolinecarboxylate (Intermediate 69) and the procedure described for Example 31.

Mass spectrum: Found [MH⁺] 452,454

H.p.l.c. Rₜ 2.52 min

¹H NMR (DMSO-d₆) δ: 1.57 (3H, d); 1.86-1.99 (1H, m); 2.43-2.52 (1H, m); 2.92-3.11 (2H, m); 3.27-3.44 (2H, m); 3.70-3.76 (2H, m); 4.21-4.30 (1H, m); 4.47-4.77 (1H, m); 6.99 (1H, d); 7.20 (1H, d); 7.32 (1H, d); 7.42-7.45 (2H, m); 7.50 (1H, d); 7.60 (1H, dd); 8.02 (1H, d); 9.21 (1H, br s); 9.65 (1H, br s).

### Example 33

### (E)-2-(5-Chloro-2-thienyl)-N-[(3S)-1-(3-methyl-1,2,3,4-tetrahydro-6-isoquinolinyl)-2-oxo-3-pyrrolidinyl]ethenesulfonamide hydrochloride

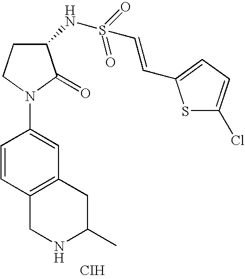

A homogeneous solution of 1,1-dimethylethyl 6-[(3S)-3-({[(E)-2-(5-chloro-2-thienyl)ethenyl]sulfonyl}amino)-2-oxo-1-pyrrolidinyl]-3-methyl-3,4-dihydro-2(1H)-isoquinolinecarboxylate (Intermediate 76) (0.049 g) in 4M HCl in dioxane (2.5 ml) containing DCM (1 ml) was stirred at room temperature for 3.5 h. During the initial 30 min, a white precipitate was formed. The mixture was evaporated to dryness, triturated with ethyl ether (3×5 ml) and dried in vacuo at 40° C. to give the title compound as a white solid (0.0338 g).

Mass spectrum: Found: MH⁺ 452/454

H.p.l.c. Rₜ 2.45 min

¹H NMR (DMSO-d₆) δ: 1.37 (3H, m), 1.93 (1H, m), 2.46 (1H excess, m), 2.83 (1H, m), 3.04 (1H, brd), 3.52 (1H, brs), 3.72 (2H, m), 4.27 (3H, m), 6.98 (1H, d), 7.20 (1H, d), 7.25 (1H, d), 7.44 (2H, m), 7.50 (1H, d,), 7.57 (1H, brd), 8.02 (1H, d), 9.35 (2H, brm).

In one aspect of the invention, a compound of Example 33 may be (E)-2-(5-Chloro-2-thienyl)-N-{(3S)-1-[(3S)-3-methyl-1,2,3,4-tetrahydro-6-isoquinolinyl]-2-oxo-3-pyrrolidinyl}ethenesulfonamide:

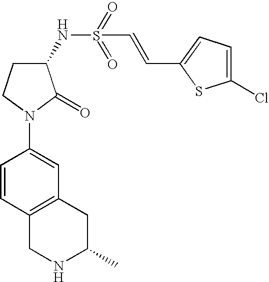

or a pharmaceutically acceptable derivative thereof.

In another aspect of the invention, a compound of Example 33 may be (E)-2-(5-Chloro-2-thienyl)-N-{(3S)-1-[(3R)-3-methyl-1,2,3,4-tetrahydro-6-isoquinolinyl]-2-oxo-3-pyrrolidinyl}ethenesulfonamide:

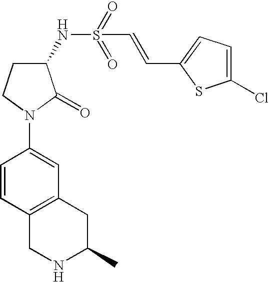

or a pharmaceutically acceptable derivative thereof.

### Example 34

### (E)-2-(5-Chloro-2-thienyl)-N-methyl-N-[(3S)-2-oxo-1-(1,2,3,4-tetrahydro-6-isoquinolinyl)-3-pyrrolidinyl]ethenesulfonamide hydrochloride

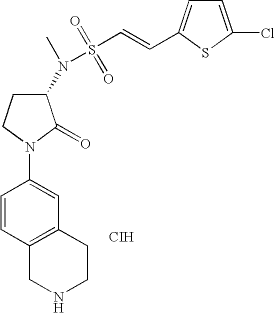

1,1-Dimethylethyl 6-{(3S)-3-[{[(E)-2-(5-chloro-2-thienyl)ethenyl]sulfonyl}(methyl)amino]-2-oxo-1-pyrrolidinyl}-3,4-dihydro-2(1H)-isoquinolinecarboxylate (Intermediate 81) (36 mg) was dissolved in 4M hydrogen chloride in 1,4-dioxane (2 ml) and the solution was left at room temperature overnight. The solvent and excess of hydrogen chloride were blown off using a stream of nitrogen to give the title compound (23 mg) as a pale yellow solid.

¹H NMR (DMSO-d₆) δ: 2.12-2.24 (1H, m); 2.31-2.41 (1H, m); 2.71 (3H, s); 3.01 (2H, t); 3.30-3.40 (2H, m, obscured by HOD signal); 3.70-3.83 (2H, m); 4.20 (2H, br. s); 4.85 (1H, dd); 6.95 (1H, d); 7.20-7.26 (2H, m); 7.46-7.57 (4H, m); 9.35 (2H, br. s)

Mass spectrum: Found [MH⁺]⁻ 452, 454

H.p.l.c. Rₜ 2.52 min

### Example 35

### (E)-2-(5-Chloro-2-thienyl)-N-[(3S)-2-oxo-1-(4,5,6,7-tetrahydro[1,3]thiazolo[5,4-c]pyridin-2-yl)-3-pyrrolidinyl]ethenesulfonamide hydrochloride

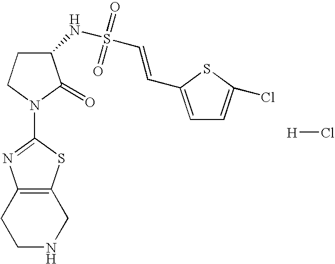

The title compound was prepared using 1,1-dimethylethyl 2-[(3S)-3-({[(E)-2-(5-chloro-2-thienyl)ethenyl]sulfonyl}amino)-2-oxo-1-pyrrolidinyl]-6,7-dihydro[1,3]thiazolo[5,4-c]pyridine-5(4H)-carboxylate (Intermediate 80) and the procedure described for Example 16.

Mass spectrum: Found: MH⁺ 444, 446

H.p.l.c. Rₜ 2.39 min

¹H NMR (MeOD-d₃) δ: 2.06-2.19 (1H, m); 2.64-2.73 (1H, m); 3.00-3.06 (2H, m); 3.55-3.61 (2H, m); 3.85-3.91 (1H, m); 4.15-4.22 (1H, m); 4.39-4.43 (2H, m); 4.49 (1H, dd); 6.90 (1H, d); 7.01 (1H, d); 7.22 (1H, d); 7.50 (1H, d)

### Example 36

### 5-Chloro-N-[(3S)-2-oxo-1-(1,2,3,4-tetrahydro-6-isoquinolinyl)-3-pyrrolidinyl]-1H-indole-2-sulfonamide

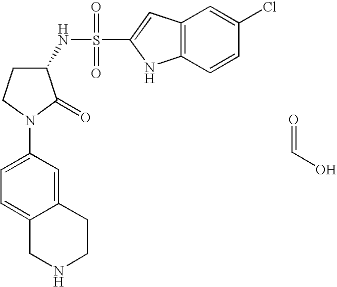

The title compound was prepared using 1,1-dimethylethyl 6-((3S)-3-{[(5-chloro-1-{[(1,1-dimethylethyl)oxy]carbonyl}-1H-indol-2-yl)sulfonyl]amino}-2-oxo-1-pyrrolidinyl)-3,4-dihydro-2(1H)-isoquinolinecarboxylate (Intermediate 83) following a similar procedure to that described for Example 6.

Mass spectrum: Found: MH⁺ 445

H.p.l.c. Rₜ min 2.52 min

¹H NMR (MeOD-d₃) δ: 1.97 (1H, m), 2.46 (1H, m), 3.09 (2H, t), 3.47 (2H, t), 3.77 (2H, m), 4.31 (2H, br. s), 4.38 (1H, dd), 7.05 (1H, s), 7.21 (1H, d), 7.26 (1H, dd), 7.44 (1H, d), 7.51 (2H, m), 7.65 (1H, d), 8.40 (1H, br.s)

### Example 37

### (E)-2-(4-Chlorophenyl)-N-[(3S)-1-(2-methyl-1,2,3,4-tetrahydro-6-isoquinolinyl)-2-oxo-3-pyrrolidinyl]ethenesulfonamide formate

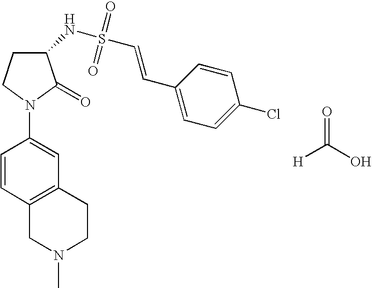

The title compound was prepared using (3S)-3-amino-1-(2-methyl-1,2,3,4-tetrahydro-6-isoquinolinyl)-2-pyrrolidinone (Intermediate 15) and (E)-2-(4-chlorophenyl)ethenesulfonyl chloride following the procedure described for Intermediate 7. Purification was carried out by mass directed preparative hplc and freeze drying from dioxane.

Mass spectrum: Found: MH⁺ 446

H.p.l.c. Rₜ 2.42 min

¹H NMR (MeOD-d₃) δ: 2.07 (1H, m), 2.61 (1H, m), 2.96 (3H, s), 3.16 (2H, t) 3.46 (2H, t), 3.81 (2H, m), 4.33 (3H, m), 7.20 (2H, m), 7.41-7.48 (3H, m), 7.53-7.61 (4H, m), 8.30 (2H, brs).

### Example 38

### 6-Chloro-N-[(3S)-1-(3-methyl-1,2,3,4-tetrahydro-6-isoquinolinyl)-2-oxo-3-pyrrolidinyl]-2-naphthalenesulfonamide formate

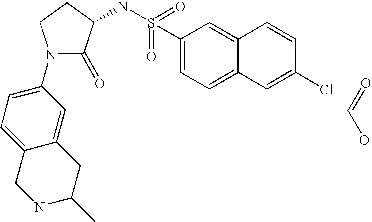

A solution of 1,1-dimethylethyl 6-((3S)-3-{[(6-chloro-2-naphthalenyl)sulfonyl]amino}-2-oxo-1-pyrrolidinyl)-3-methyl-3,4-dihydro-2(1H)-isoquinolinecarboxylate (Intermediate 82) (0.072 g) in 4M HCl in dioxane (4 ml) was stirred at room temperature. During the initial 30 min a white solid was formed. Sufficient MeOH was added to dissolve the solid and after a further 2 h, the mixture was evaporated to dryness. The resultant white solid was quenched with aqueous sodium hydrogen carbonate (10 ml), then extracted with DCM and passed through a hydrophobic frit. Evaporation in vacuo gave a gum which was purified by mass directed preparative hplc to provide, after freeze drying, the title compound (0.058 g) as a white solid.

Mass spectrum: Found: MH⁺ 470, 472

H.p.l.c. Rₜ 2.63 min

¹H-NMR (MeOD-d₃) δ: 1.45 (3H, d), 1.92 (1H, m), 2.40 (1H, m), 2.85 (1H, m), 3.10 (1H, dt), 3.59 (1H, m), 3.72 (2H, m), 4.32 (1H, m), 4.34 (2H, s), 7.20 (1H, d), 7.46 (2H, m), 7.61 (1H, dd), 8.02 (4H, m), 8.44 (1H, s), 8.54 (1H, s).

### In Vitro Assay for Inhibition of Factor Xa

Compounds of the present invention were tested for their Factor Xa inhibitory activity as determined in vitro by their ability to inhibit human Factor Xa in a fluorogenic assay, using Rhodamine 110, bis-Cbz-glycylglycyl-L-arginine amide as the fluorogenic substrate. Compounds were diluted from a 10 mM stock solution in dimethylsulfoxide at appropriate concentrations. Assay was performed at room temperature using buffer consisting of: 50 mM Tris-HCl, 150 mM NaCl, 5 mM CaCl₂, pH 7.4 containing human Factor Xa (final concentration of 0.0003 U.ml-1). Compound and enzyme were preincubated for 15 min prior to addition of the substrate (final concentration of 10 μM). The reaction was stopped after 3 hrs with the addition of H-D-Phe-Pro-Arg-Chloromethylketone. An LJL-Analyst fluorimeter was used to monitor fluorescence with 485 nm excitation/535 nm emission. To obtain IC₅₀ values the data were analysed using ActivityBase® and XLfit®.

### Calculation of Ki Values:

Ki=IC₅₀/(1+[Substrate]/Km)

The Ki value for the above assay can be obtained by dividing the IC₅₀ value by 1.6.

All of the synthetic Example compounds tested by the above described in vitro assay (Examples 1, 2, 3, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38) were found to exhibit Factor Xa inhibitory activity. Preferably, compounds have a Ki value of less than 1 μM. More preferably, compounds have a Ki value of less than 0.1 μM. Most preferably, compounds have a Ki value of less than 10 nM, e.g. Examples 1, 2, 3, 4, 6, 7, 8, 17, 18, 19, 20, 21, 22, 23, 26, 30, 31, 32, 33, 34, 36.

### Method for Measurement of Prothrombin Time (PT)

Blood was collected into a sodium citrate solution (ratio 9:1) to give a final concentration of 0.38% citrate. Plasma was generated by centrifugation of citrated blood samples at 1200×g for 20 min at 4° C. and stored at −20° C. until use. PT analysis was conducted using plasma pooled from 4 separate donors (2 male and 2 female).

The PT test was performed using the BCS Coagulation Analyzer (Dade Behring). For assay, 50 ul of plasma containing test compound at concentrations ranging from 0.03 to 100 uM (made from a 100 uM stock containing 1% DMSO in plasma) was combined with 100 ul of Thromboplastin C Plus (Dade Behring). Upon addition of the reagents, absorbance at 405 nm was monitored and time to clot formation is determined (normal range for human plasma is 10.6-12.4 seconds).

Examples 1, 2, 3, 4, 5, 6, 7, 8, 9, 10, 11, 12, 13, 14, 16, 17, 18, 19, 20, 21, 22, 23, 24, 25, 26, 27, 28, 29, 30, 31, 32, 33, 36, 37, 38 have been shown to exhibit activity.

### General Purification and Analytical Methods

### LC/MS Method

Analytical HPLC was conducted on a Supelcosil LCABZ+PLUS column (3 μm, 3.3 cm×4.6 mm ID) eluted with 0.1% HCO₂H and 0.01M ammonium acetate in water (solvent A), and 95% MeCN and 0.05% HCO₂H in water (solvent B), using the following elution gradient 0-0.7 min 0% B, 0.7-4.2 min 0→100% B, 4.2-5.3 min 100% B, 5.3-5.5 min 100→0% B at a flow rate of 3 ml/min (System 1). The mass spectra (MS) were recorded on a Fisons VG Platform mass spectrometer using electrospray positive ionisation [(ES+ve to give MH⁺ and M(NH₄)⁺ molecular ions] or electrospray negative ionisation [(ES−ve to give (M-H)⁻ molecular ion] modes.

¹H nmr spectra were recorded using a Bruker DPX 400 MHz spectrometer using tetramethylsilane as the external standard.

Biotage™ chromatography refers to purification carried out using equipment sold by Dyax Corporation (either the Flash 40i or Flash 150i) and cartridges pre-packed with KPSil™.

Mass directed preparative h.p.l.c. refers to methods where the material was purified by high performance liquid chromatography on a HPLCABZ+5 μm column (5 cm×10 mm internal diameter) with 0.1% HCO₂H in water and 95% MeCN, 5% water (0.5% HCO₂H) utilising the following gradient elution conditions: 0-1.0 min 5% B, 1.0-8.0 min 5→30% B, 8.0-8.9 min 30% B, 8.9-9.0 min 30→95% B, 9.0-9.9 min 95% B, 9.9-10 min 95→0% B at a low rate of 8 ml min⁻¹(System 2). The Gilson 202-fraction collector was triggered by a VG Platform Mass Spectrometer on detecting the mass of interest.

Hydrophobic frits refers to filtration tubes sold by Whatman.

SPE (solid phase extraction) refers to the use of cartridges sold by International Sorbent Technology Ltd. Silica SPE and SCX SPE were used.

Combi Flash® Companion™ refers to an automated purification system sold by ISCO Inc.

Redisep® silica columns refer to pre-packed columns sold by ISCO Inc.

### TFA System:

Preparative h.p.l.c. (Autoprep HPLC or Autoprep) refers to methods where the material was purified by high performance liquid chromatography on a Supelcosil ABZ+5 μm column (10 cm×21.2 mm i.d.) with a suitable gradient of 0.1% trifluoroacetic acid in water and MeCN (with 0.5% trifluoroacetic acid). The Gilson 233 fraction collector was triggered by UV (254 nm or a more suitable wavelength if appropriate).

1. At least one chemical entity chosen from compounds of formula I: 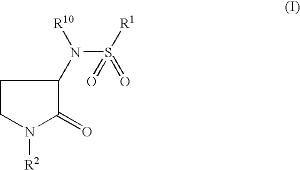
wherein:
R¹ represents a group selected from: 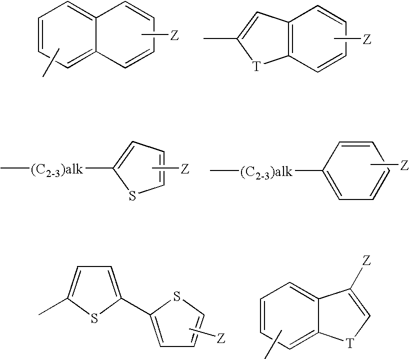
each ring of which optionally contains a further heteroatom N,
Z represents an optional substituent halogen, —C₁₋₃alkyl or —NR^{a}R^{b},
alk represents alkylene or alkenylene,
T represents S, O or NH;
R^{a} and R^{b} independently represent hydrogen or —C₁₋₃alkyl;
R² represents a group selected from: 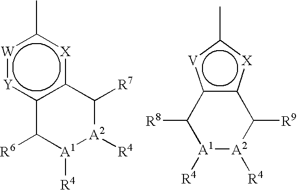
W, X and Y independently represent CH, C—R⁵ or N;
R⁵ represents halogen or C₁₋₃alkyl;
V represents NR³, S or O;
R³ represents hydrogen or C₁₋₃alkyl;
one of A¹ and A² represents N and the other represents CH;
Each R⁴, R⁶, R⁷, R⁸, R⁹ independently represents hydrogen or C₁₋₃alkyl;
R¹⁰ represents hydrogen, —C₁₋₆alkyl, —C₁₋₃alkylCONR^{a}R^{b}, —C₁₋₆alkylCO₂C₁₋₄alkyl, —CO₂C₁₋₄alkyl or —C₁₋₃alkylCO₂H;
and pharmaceutically acceptable derivative(s) thereof.

2. At least one chemical entity according to claim 1 wherein R¹ represents a group:

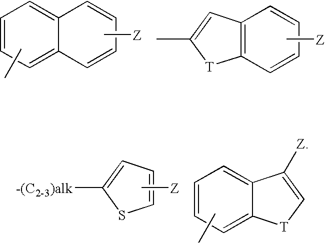

3. At least one chemical entity according to claim 1 or claim 2 wherein R² represents a group:

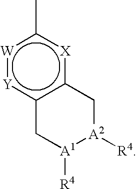

4. At least one chemical entity according to any one of claims 1-3 wherein Z represents chloro.

5. At least one chemical entity according to claim 1 selected from the list:
6-Chloro-N-[(3S)-2-oxo-1-(1,2,3,4-tetrahydro-6-isoquinolinyl)-3-pyrrolidinyl]-2-naphthalenesulfonamide;
(E)-2-(5-Chloro-2-thienyl)-N-[(3S)-2-oxo-1-(1,2,3,4-tetrahydro-6-isoquinolinyl)-3-pyrrolidinyl]ethenesulfonamide;
6-Chloro-N-[(3S)-2-oxo-1-(1,2,3,4-tetrahydro-6-isoquinolinyl)-3-pyrrolidinyl]-1-benzothiophene-2-sulfonamide;
3-Chloro-N-[(3S)-2-oxo-1-(1,2,3,4-tetrahydro-6-isoquinolinyl)-3-pyrrolidinyl]-1H-indole-6-sulfonamide;
N-[(3S)-2-Oxo-1-(1,2,3,4-tetrahydro-6-isoquinolinyl)-3-pyrrolidinyl]-1H-indole-6-sulfonamide;
6-Chloro-N-[(3S)-2-oxo-1-(1,2,3,4-tetrahydro-6-isoquinolinyl)-3-pyrrolidinyl]-1H-indole-2-sulfonamide;
(E)-2-(5-Chloro-2-thienyl)-N-[(3S)-1-(2-methyl-1,2,3,4-tetrahydro-6-isoquinolinyl)-2-oxo-3-pyrrolidinyl]ethenesulfonamide;
6-Chloro-N-[(3S)-1-(2-methyl-1,2,3,4-tetrahydro-6-isoquinolinyl)-2-oxo-3-pyrrolidinyl]-1-benzothiophene-2-sulfonamide;
5-Chloro-N-[(3S)-2-oxo-1-(1,2,3,4-tetrahydro-6-isoquinolinyl)-3-pyrrolidinyl]-1-benzothiophene-2-sulfonamide;
5′-Chloro-N-[(3S)-2-oxo-1-(1,2,3,4-tetrahydro-6-isoquinolinyl)-3-pyrrolidinyl]-2,2′-bithiophene-5-sulfonamide;
6-Chloro-N-[(3S)-2-oxo-1-(1,2,3,4-tetrahydro-7-isoquinolinyl)-3-pyrrolidinyl]-2-naphthalenesulfonamide;
2-(5-Chloro-2-thienyl)-N-[(3S)-2-oxo-1-(1,2,3,4-tetrahydro-7-isoquinolinyl)-3-pyrrolidinyl]ethanesulfonamide;
(E)-2-(5-Chloro-2-thienyl)-N-[(3S)-2-oxo-1-(1,2,3,4-tetrahydro-7-isoquinolinyl)-3-pyrrolidinyl]ethenesulfonamide;
3-Chloro-N-[(3S)-2-oxo-1-(1,2,3,4-tetrahydro-7-isoquinolinyl)-3-pyrrolidinyl]-1H-indole-6-sulfonamide;
N-[(3S)-2-Oxo-1-(1,2,3,4-tetrahydro-7-isoquinolinyl)-3-pyrrolidinyl]-1H-indole-6-sulfonamide;
6-Chloro-N-[(3R)-2-oxo-1-(1,2,3,4-tetrahydro-6-isoquinolinyl)-3-pyrrolidinyl]-2-naphthalenesulfonamide;
6-Chloro-N-[(3S)-1-(5-fluoro-1,2,3,4-tetrahydro-6-isoquinolinyl)-2-oxo-3-pyrrolidinyl]-2-naphthalenesulfonamide;
(E)-2-(5-Chloro-2-thienyl)-N-[(3S)-1-(5-fluoro-1,2,3,4-tetrahydro-6-isoquinolinyl)-2-oxo-3-pyrrolidinyl]ethenesulfonamide;
3-Chloro-N-[(3S)-1-(5-fluoro-1,2,3,4-tetrahydro-6-isoquinolinyl)-2-oxo-3-pyrrolidinyl]-1H-indole-6-sulfonamide;
6-Chloro-N-[1-(7-fluoro-1,2,3,4-tetrahydro-6-isoquinolinyl)-2-oxo-3-pyrrolidinyl]-2-naphthalenesulfonamide;
6-Chloro-N-[(3S)-1-(7-fluoro-1,2,3,4-tetrahydro-6-isoquinolinyl)-2-oxo-3-pyrrolidinyl]-1-benzothiophene-2-sulfonamide;
(E)-2-(5-Chloro-2-thienyl)-N-[(3S)-1-(7-fluoro-1,2,3,4-tetrahydro-6-isoquinolinyl)-2-oxo-3-pyrrolidinyl]ethenesulfonamide;
3-Chloro-N-[(3S)-1-(7-fluoro-1,2,3,4-tetrahydro-6-isoquinolinyl)-2-oxo-3-pyrrolidinyl]-1H-indole-6-sulfonamide;
6-Chloro-N-[(3S)-1-(7-chloro-1,2,3,4-tetrahydro-6-isoquinolinyl)-2-oxo-3-pyrrolidinyl]-2-naphthalenesulfonamide;
(E)-N-[(3S)-1-(7-Chloro-1,2,3,4-tetrahydro-6-isoquinolinyl)-2-oxo-3-pyrrolidinyl]-2-(5-chloro-2-thienyl)ethenesulfonamide;
3-Chloro-N-[(3S)-1-(7-chloro-1,2,3,4-tetrahydro-6-isoquinolinyl)-2-oxo-3-pyrrolidinyl]-1H-indole-6-sulfonamide;
6-Chloro-N-[(3S)-1-(7-methyl-1,2,3,4-tetrahydro-6-isoquinolinyl)-2-oxo-3-pyrrolidinyl]-2-naphthalenesulfonamide;
(E)-2-(5-Chloro-2-thienyl)-N-[(3S)-1-(7-methyl-1,2,3,4-tetrahydro-6-isoquinolinyl)-2-oxo-3-pyrrolidinyl]ethenesulfonamide;
3-Chloro-N-[(3S)-1-(7-methyl-1,2,3,4-tetrahydro-6-isoquinolinyl)-2-oxo-3-pyrrolidinyl]-1H-indole-6-sulfonamide;
6-Chloro-N-[(3S)-1-(1-methyl-1,2,3,4-tetrahydro-6-isoquinolinyl)-2-oxo-3-pyrrolidinyl]-2-naphthalenesulfonamide;
(E)-2-(5-Chloro-2-thienyl)-N-[(3S)-1-(1-methyl-1,2,3,4-tetrahydro-6-isoquinolinyl)-2-oxo-3-pyrrolidinyl]ethenesulfonamide;
(E)-2-(5-Chloro-2-thienyl)-N-[(3S)-1-(3-methyl-1,2,3,4-tetrahydro-6-isoquinolinyl)-2-oxo-3-pyrrolidinyl]ethenesulfonamide;
(E)-2-(5-Chloro-2-thienyl)-N-methyl-N-[(3S)-2-oxo-1-(1,2,3,4-tetrahydro-6-isoquinolinyl)-3-pyrrolidinyl]ethenesulfonamide;
(E)-2-(5-Chloro-2-thienyl)-N-[(3S)-2-oxo-1-(4,5,6,7-tetrahydro[1,3]thiazolo[5,4-c]pyridin-2-yl)-3-pyrrolidinyl]ethenesulfonamide;
5-Chloro-N-[(3S)-2-oxo-1-(1,2,3,4-tetrahydro-6-isoquinolinyl)-3-pyrrolidinyl]-1H-indole-2-sulfonamide;
(E)-2-(4-Chlorophenyl)-N-[(3S)-1-(2-methyl-1,2,3,4-tetrahydro-6-isoquinolinyl)-2-oxo-3-pyrrolidinyl]ethenesulfonamide;
6-Chloro-N-[(3S)-1-(3-methyl-1,2,3,4-tetrahydro-6-isoquinolinyl)-2-oxo-3-pyrrolidinyl]-2-naphthalenesulfonamide;
and pharmaceutically acceptable derivatives thereof.

6. At least one chemical entity according to any one of claims 1-5 for use in therapy.

7. A pharmaceutical composition comprising at least one chemical entity according to any one of claims 1-5 together with at least one pharmaceutical carrier and/or excipient.

8. Use of at least one chemical entity according to any one of claims 1-5 for the manufacture of a medicament for the treatment of a patient suffering from a condition susceptible to amelioration by a Factor Xa inhibitor.

9. A method of treating a patient suffering from a condition susceptible to amelioration by a Factor Xa inhibitor comprising administering a therapeutically effective amount of at least one chemical entity according to any one of claims 1-5.

10. A process for preparing a compound of formula (I) which comprises reacting compounds of formula (II) or an acid addition salt thereof with compounds of formula (III): 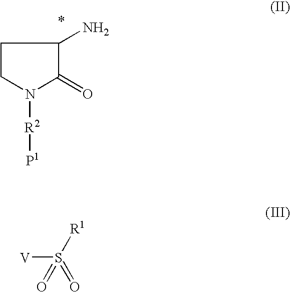
wherein:
R¹ represents a group selected from: 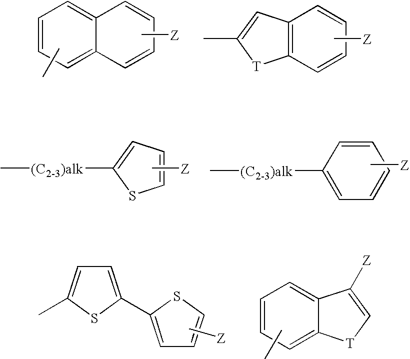
each ring of which optionally contains a further heteroatom N,
Z represents an optional substituent halogen, —C₁₋₃alkyl or —NR^{a}R^{b},
alk represents alkylene or alkenylene,
represents S, O or NH;
R^{a} and R^{b} independently represent hydrogen or —C₁₋₃alkyl;
R² represents a group selected from: 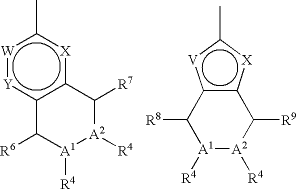
W, X and Y independently represent CH, C—R⁵ or N;
R⁵ represents halogen or C₁₋₃alkyl;
V represents NR³, S or O;
R³ represents hydrogen or C₁₋₃alkyl;
one of A¹ and A² represents N and the other represents CH;
Each R⁴, R⁶, R⁷, R⁸, R⁹ independently represents hydrogen or C₁₋₃alkyl; R¹⁰ represents hydrogen, —C₁₋₆alkyl, —C₁₋₃alkylCONR^{a}R^{b}, —C₁₋₃alkylCO₂C₁₋₄alkyl, —CO₂C₁₋₄alkyl or —C₁₋₃alkylCO₂H; and V represents a suitable leaving group, optionally followed by removal of P¹ under standard conditions.
